(19) United States
(12) Patent Application Publication
(10) Pub. No.: US 2019/0194386 A1
(43) Pub. Date: Jun. 27, 2019

(54) Conducting Polymers and Uses Thereof

(71) Applicant: Auckland UniServices Limited, Auckland (NZ)

(72) Inventors: Jadranka Travas-Sejdic, Auckland (NZ); David Edward Williams, Auckland (NZ); Nihan Aydemir, Auckland (NZ); David Barker, Auckland (NZ); Wai Chi Eddie Chan, Auckland (NZ); Clive William Evans, Auckland (NZ)

(21) Appl. No.: 16/330,833

(22) PCT Filed: Sep. 8, 2017
(86) PCT No.: PCT/IB2017/055408

§ 371 Date: Mar. 6, 2019

(30) Foreign Application Priority Data
Sep. 9, 2016 (NZ) 724050

(51) Int. Cl. C08G 61/12; C07D 207/323; C07D 333/08; C12Q 1/6844; B01L 7/00; C12Q 1/6862; C07D 333/16; C07D 207/333; G01N 27/327

(52) U.S. Cl. CPC C08G 61/126; C25D 9/02; C07D 207/323; C07D 333/08; C12Q 1/6844; B01L 7/52; C12Q 1/6862; C07D 333/16; C07D 207/333; G01N 27/3277; C08G 2261/12; C08G 2261/1424; C08G 2261/1426; C08G 2261/1452; C08G 2261/312; C08G 2261/3221; C08G 2261/3223; C08G 2261/44; C08G 2261/94; B01L 2300/0663; B01L 2300/0645; C08G 61/124

(57) ABSTRACT

The present invention generally relates to the field of conducting polymers. More specifically, the present invention relates to polymerisable monomers comprising a probe capable of binding one or more nucleic acids or comprising a nucleic acid or an analogue thereof, conducting polymers comprising monomer units of such monomers, and methods of making such polymers. The present invention also relates to sensors comprising the polymers, sensor systems comprising the sensors, methods of making the sensors, and methods for determining the presence or absence or amount of targets employing the sensors. The present invention also relates to methods, systems and apparatuses for amplifying nucleic acids employing the conducting polymers.

### FIELD OF THE INVENTION

The present invention generally relates to the field of conducting polymers. More specifically, the present invention relates to polymerisable monomers comprising a probe capable of binding one or more nucleic acids or comprising a nucleic acid or an analogue thereof, conducting polymers comprising monomer units of such monomers, and methods of making such polymers. The present invention also relates to sensors comprising the polymers, sensor systems comprising the sensors, methods of making the sensors, and methods for determining the presence or absence or amount of targets employing the sensors. The present invention also relates to methods, systems and apparatuses for amplifying nucleic acids employing the conducting polymers.

BACKGROUND TO THE INVENTION

Over the last two decades the use of biosensors in the detection of biological targets such as nucleic acids has become a rapidly-expanding area of research worldwide.

Biosensors have potential applications in a number of fields including drug delivery, biomedical devices and medical diagnostics. Improvements in the understanding of sensor-target interactions have allowed for the preparation of improved sensor systems for use in such applications. However, many sensors are still limited by their sensitivity, selectivity, ease-of-preparation and/or ease-of-use.

There is an ongoing need for sensors capable of detecting targets such as nucleic acids. It is an object of the present invention to go some way to meeting this need; and/or at least provide the public with a useful choice.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY OF THE INVENTION

In a first aspect the present invention broadly consists in a polymerisable monomer of formula (1):

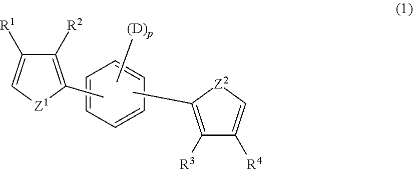

wherein

p is 1 or 2;

R¹, R², R³ and R⁴ are each independently selected from the group consisting of hydrogen, an electron withdrawing group and an electron donating group; or

R¹ and R² together and/or R³ and R⁴ together represent an electron withdrawing group or an electron donating group that together with the atoms to which they are attached form a five or six membered ring;

D at each instance of p is independently a group of the formula -L-P^{x}, wherein L is a bond or a linker group, and P^{x} is a probe capable of binding one or more nucleic acids or comprising a nucleic acid or an analogue thereof;

Z¹ and Z² are each independently S or NR^{a}; and

R^{a} at each instance is independently selected from the group consisting of hydrogen and alkyl.

In a second aspect the present invention broadly consists in a conducting polymer comprising a monomer unit of the formula (2):

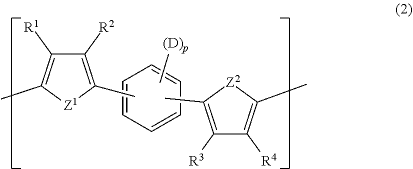

wherein p, R¹, R², R³, R⁴, D, Z¹, and Z² are as defined in the first aspect.

In a third aspect the invention broadly consists in a method of making a conducting polymer as defined in the second aspect, the method comprising:

(a) providing a polymerisable monomer of the formula (1) as defined in the first aspect, and

(b) polymerising the monomer to provide a conducting polymer as defined in the second aspect.

In a fourth aspect the present invention broadly consists in a conducting polymer made by a method as defined in the third aspect.

In a fifth aspect the present invention broadly consists in a method of making a sensor comprising:

(i) providing a monomer of the formula (1) as defined in the first aspect;

(ii) providing a substrate; and

(iii) polymerising the monomer of the formula (1) as defined in the first aspect to provide a conducting polymer as defined in the second aspect and depositing the conducting polymer on a surface of the substrate to provide a coating of the conducting polymer on the surface of the substrate; or

(iii) depositing the monomer of the formula (1) as defined in the first aspect on a surface of the substrate and polymerising the monomer to provide a coating of a conducting polymer as described in the second aspect on the surface of the substrate.

In a sixth aspect the present invention broadly consists in a sensor comprising a substrate having a surface coated with a conducting polymer as defined in the second aspect.

In a seventh aspect the present invention broadly consist in a sensor system comprising a sensor as defined in the sixth aspect and a detector for determining the presence or absence or amount of a target, for example a detector capable of detecting binding of a target by a probe.

In an eighth aspect the present invention broadly consists in a method for amplifying a target nucleic acid, the method comprising the steps of
a) providing a reaction volume comprising
   (i) a first electrode comprising an electrochemically-active conducting polymer as defined in the second aspect, wherein the monomer unit of the formula (2) in the conducting polymer comprises a first single-stranded nucleic acid molecule capable of hydridizing to a first portion of a target nucleic acid sequence , and
   (iii) a second electrode;
b) providing a reaction mixture to the reaction volume, the reaction mixture comprising
   (i) a sample comprising the target nucleic acid,
   (ii) a second single-stranded nucleic acid molecule comprising a nucleic acid sequence complementary to a second portion of the target nucleic acid sequence,
   (iii) a nucleic acid polymerase,
   (iv) a redox couple, and
   (v) a supply of reagents for a nucleic acid amplification reaction;
c) performing a polymerase chain reaction, and
d) measuring the impedance of the first electrode at least once during the polymerase chain reaction.

In a ninth aspect the present invention broadly consists in an apparatus for real-time nucleic acid amplification, the apparatus comprising
a reaction volume comprising
(i) a first electrode comprising an electrochemically-active conducting polymer as defined in the second aspect, wherein the the monomer unit of the formula (2) in the conducting polymer comprises a first single-stranded nucleic acid molecule capable of hydridizing to a first portion of a target nucleic acid sequence, and
(iii) a second electrode;
wherein the reaction volume is suitable for containing a sample comprising nucleic acid, and wherein the reaction volume includes a heater or is adapted to engage with a thermocycler suitable for PCR.

In a tenth aspect the present invention broadly consists in a system for amplifying a target nucleic acid in a sample, the system comprising
a) a reaction volume comprising
   (i) a first electrode comprising an electrochemically-active conducting polymer as defined in the second aspect, wherein the the monomer unit of the formula (2) in the conducting polymer comprises a first single-stranded nucleic acid molecule capable of hydridizing to a first portion of a target nucleic acid sequence, and
   (iii) a second electrode;
b) optionally a reaction mixture comprising one or more of
   (i) a second single-stranded nucleic acid molecule comprising a nucleic acid sequence complementary to a second portion of the target nucleic acid sequence,
   (iii) a nucleic acid polymerase,
   (iv) a redox couple, and
   (v) a supply of reagents for a nucleic acid amplification reaction;
c) a device for measuring the impedance of at least the first electrode; and
d) a thermocycler.

In an eleventh aspect the present invention broadly consists in a method for determining the presence or absence or amount of a target in a sample, the method comprising:

(a) contacting
(1) a sample which may comprise a target, and
(2) a sensor as defined in the sixth aspect or a sensor system as defined in the seventh aspect; and

(b) determining the presence or absence or amount of the target in the sample.

In a twelfth aspect, the present invention broadly consists in a sensor made by a method according to the fifth aspect.

The following embodiments and preferences may relate alone or in any combination of any two or more to any of the above aspects.

In some embodiments the polymerisable monomer has the formula (1A):

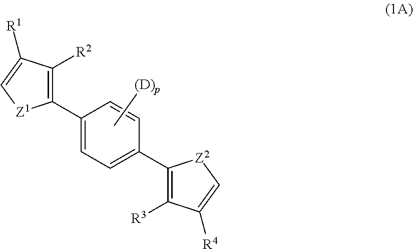

wherein p, R¹, R², R³, R⁴, D, Z¹, and Z² are as defined herein. In some embodiments the polymerisable monomer has the structure (1B):

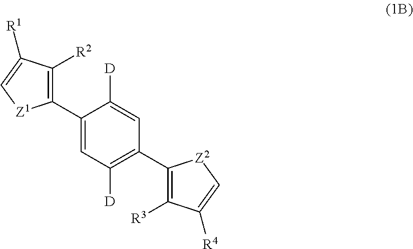

wherein R¹, R², R³, R⁴, D, Z¹, and Z² are as defined herein

In some embodiments R¹, R², R³, and R⁴ are each independently selected from the group consisting of hydrogen, halo, nitro, nitrile, —C(O)R⁵, —OR⁵, —C(O)OR⁵, —OC(O)R⁵, —NR⁵R⁵, —C(O)NR⁵R⁵, —NR⁵C(O)R⁵, —NR⁵C(O)NR⁵R⁵, and —R⁶; or

R¹ and R² and/or R³ and R⁴ together with the atoms to which they are attached form a five or six membered heterocyclic or carbocyclic ring;

R⁵ at each instance is independently selected from the group consisting of hydrogen, alkyl, alkenyl, cycloalkyl, cycloalkenyl, aryl, heterocyclyl, and heteroaryl; and

R⁶ at each instance is independently selected from the group consisting of alkyl, alkenyl, cycloalkyl, cycloalkenyl, aryl, arylalkyl, heterocyclyl, and heteroaryl, each of which is optionally substituted with one or more substituents independently selected from halo, nitro, nitrile, —C(O)R⁵, —OR⁵, —C(O)OR⁵, —OC(O)R⁵, —NR⁵R⁵, —C(O)NR⁵R⁵, —NR⁵C(O)R⁵, —NR⁵C(O)NR⁵R⁵, and alkyl.

In some embodiments R¹, R², R³, and R⁴ are each independently selected from the group consisting of hydrogen, alkyl and alkoxy; or R¹ and R² and/or R³ and R⁴ together represent —OCH₂CH₂O—. In some embodiments R¹, R², R³, and R⁴ are each hydrogen; or R¹ and R² together and/or R³ and R⁴ together represent —OCH₂CH₂O—.

In certain embodiments R¹ and R⁴ are identical and R² and R³ are identical; or when R¹ and R² form a ring and R³ and R⁴ form a ring, each ring is identical.

In certain embodiments R¹, R², R³, and R⁴ are each hydrogen.

In exemplary embodiments Z¹ and Z² are each S; or Z¹ and Z² are each NRa.

In certain embodiments R^{a} at each instance is hydrogen.

In certain embodiments p is 2.

In some embodiments the polymerisable monomer has the formula (1C) or (1D):

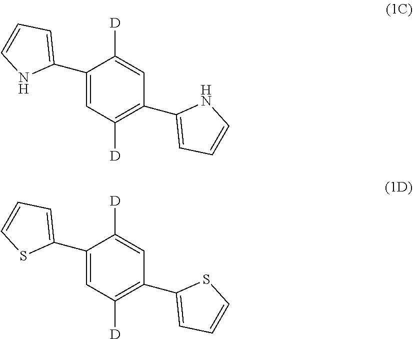

wherein D is as defined herein.

In certain embodiments each D is identical.

In certain embodiments the polymerisable monomer has an oxidation potential for polymerisation of from about 0 to about 1.0V vs. Ag/AgCl (3 M KCl), for example from about 0.2 to 1.0, 0.3 to 1.0, 0.4 to 1.0, 0.5 to 1.0, 0.6 to 1.0, 0.7 to 1.0, 0.8 to 1.0, 0.2 to 0.9, 0.3 to 0.9, 0.4 to 0.9, 0.5 to 0.9, 0.6 to 0.9, 0.7 to 0.9, or 0.8 to 0.9V vs. Ag/AgCl (3 M KCl).

In certain embodiments, the polymerisable monomer has an oxidation potential of from about 0.6 to 1.0V vs. Ag/AgCl (3 M KCl).

In some embodiments, the length of the linker group is from about 1 to 15 atoms. For example from about 1 to 14, 1 to 13, 1 to 12, 1 to 11, 1 to 10, 2 to 10, 3 to 10, 4 to 10, 5 to 10, 6 to 10, 7 to 10, 8 to 10, 9 to 10, 2 to 9, 3 to 9, 5 to 9, 7 to 9, 2 to 8, 4 to 8, 6 to 8, 2 to 7, 3 to 7, 5 to 7, 2 to 6, 4 to 6, 2 to 5, 3 to 5, or 2 to 4 atoms.

In some embodiments the linker group has the formula:

—X¹—[(CH₂)ₘ—X²]ₓ—(CH₂)ₙ—X³—

wherein

x is an integer from 0 to 6;

m at each instance of x is independently an integer from 0 to 8;

n is an integer from 0 to 8;

X¹ and X² at each instance of x are each independently selected from the group consisting of a bond, —CH₂—, —CH═CH—, —O—, —S—, —N(R)—, —C(O)—, —C(O)O—, —OC(O)—, —N(R)C(O)—, —C(O)N(R)—, —SC(O)—, —C(O)S—, —NRSO₂—, —SO₂NR—, and —N(R)C(O)N(R)—;

R at each instance is independently hydrogen or alkyl;

X³ is a functional group through which the probe is attached; provided that the linker group, excluding X³, is not more than 10 atoms in length.

In various embodiments, X¹ and X² at each instance of x are each independently selected from the group consisting of a bond, —CH₂—, —CH═CH—, —O—, —S—, —N(R)—, —C(O)—, —C(O)O—, —OC(O)—, —N(R)C(O)—, —C(O)N(R)—, —NRSO₂—, and —SO₂NR—.

In various embodiments, X¹ and X² at each instance of x are each independently selected from the group consisting of a bond, —CH₂—, —CH═CH—, —O—, —N(R)—, —C(O)O—, —OC(O)—, —N(R)C(O)—, and —C(O)N(R)—.

In various embodiments, X¹ and X² at each instance of x are each independently selected from the group consisting of a bond, —CH═CH—, and —O—.

In various embodiments, the linker group is of the formula:

—O—(CH₂)ₙ—X³ (a)

wherein

X³ is as defined herein; and

n is an integer from 1-8;

(b)

wherein

X³ is as defined herein;

x is an integer from 1-4; or

—O—[(CH₂)ₘ—O]ₓ—(CH₂)ₙ—X³— (c)

wherein

X³ is as defined herein;

x is an integer from 1-4;

m at each instance of x is independently an integer from 1-4, preferably 2;

n is an integer from 1-4, provided that the linker group excluding X³, is not more than 10 atoms in length.

In various embodiments, X³ is selected from the group consisting of —C(═NH)NH—, —NHC(═NH)—, —C(O)NH—, —NHC(O)—, —C(O)O—, —OC(O)—, —NHC(O)CR^{v}R^{w}S—, —SCR^{w}R^{v}C(O)NH—, —SS—, —C(O)NHN═CH—, —CH═NNHC(O)—, —CH═N—, —N═CH—,

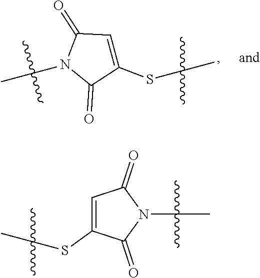

wherein R^{v} and R^{w} are at each instance independently H or C₁₋₆ alkyl, preferably H.

In certain embodiments, X³ is selected from the group consisting of —CH═N—, —N═CH—, —C(O)NH—, —NHC(O)—, —C(O)O—, and —OC(O)—.

In certain embodiments X³ is —C(O)NH—.

In some embodiments the linker group is —O—(CH₂)ₘ—C(O)NH—, wherein m is an integer from 2 to 8.

In exemplary embodiments the linker group is —O—(CH₂)₅—C(O)NH—.

In various embodiments the probe is capable of binding one or more nucleic acids in a sequence specific manner.

In certain embodiments the sequence specific binding of one or more nucleic acids by the probe is by nucleic acid hybridization.

In certain embodiments the probe comprises a single or double stranded oligonucleotide, polynucleotide, or an analogue thereof.

In various embodiments, the probe comprises a single or double stranded oligonucleotide or polynucleotide. In various embodiments, the probe comprises a single stranded oligonucleotide or polynucleotide. In various embodiments, the probe comprises a single or double stranded oligonucleotide. In certain embodiments, the probe comprises a single stranded oligonucleotide.

In certain embodiments the probe comprises an aptamer. In various embodiments, the aptamer comprises a single or double stranded oligonucleotide, polynucleotide, or an analogue thereof. In various embodiments, the aptamer comprises a single or double stranded oligonucleotide or polynucleotide. In various embodiments, the aptamer comprises a single stranded oligonucleotide or polynucleotide. In various embodiments, the aptamer comprises a single stranded oligonucleotide.

In exemplary embodiments P^{X} is an amino functionalised single stranded oligonucleotide.

In some embodiments, the or a or at least one probe comprises, consists essentially of, or consists of a single stranded oligonucleotide or polynucleotide selected from:

(SED ID NO: 1)
5′-GGTCTAGCTACAGAGAAATCTCGA-3′,
(SEQ ID NO: 2)
5′-CAGTAGACGGGGGTGTCACGCGAC-3′,
(SEQ ID NO: 3)
5′-CATCTTTGGGCTGTTTTCTTCCGC-3′,
(SEQ ID NO: 4)
5′-CTAGTTTAGACAGCTAGGAAGG-3′,

or a single stranded oligonucleotide or polynucleotide sequence comprising 7 or more (for example, 8, 10, 12, 14, or 16 or more) contiguous bases of any of SEQ ID NOs: 1 to 4.

In some embodiments the or a or at least one probe comprises, consists essentially of, or consists of a single stranded oligonucleotide or polynucleotide selected from SEQ ID NOs: 1 to 4.

In some embodiments, the or a or at least one probe comprises, consists essentially of, or consists of a single stranded oligonucleotide or polynucleotide complementary to a target single stranded oligonucleotide or polynucleotide selected from:

(SEQ ID NO: 5)
5′-TCGAGATTTCTCTGTAGCTAGACC-3′,
(SEQ ID NO: 6)
5′-TCGAGATTTCTCAGTAGCTAGACC-3′,
(SEQ ID NO: 7)
5′-TCGAGATTTCTCTCTAGCTAGACC-3′,
(SEQ ID NO: 8)
5′-GTCATCTGCCCCCACAGAGCGCTG-3′,
(SEQ ID NO: 9)
5′-GCGGAAGAAAACAGCCCAAAGATG-3′,
(SEQ ID NO: 10)
5′-CCTTCCTAGCTGTCTAAACTAG-3′,

or a target single stranded oligonucleotide or polynucleotide sequence comprising a sequence comprising 7 or more (for example, 8, 10, 12, 14, or 16 or more) contiguous bases of any of SEQ ID NOs: 5 to 10.

In some embodiments the conducting polymer comprises a monomer unit of formula (2A):

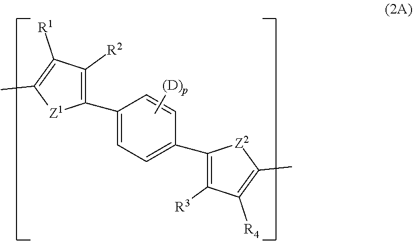

wherein p, R¹, R², R³, R⁴, D, Z¹, and Z² are as defined herein.

In various embodiments the conducting polymer comprises a monomer unit of formula (2B):

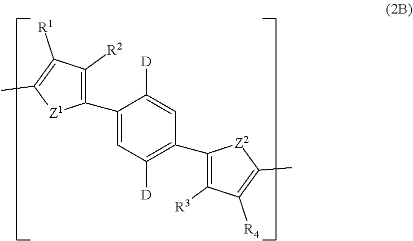

wherein R¹, R², R³, R⁴, D, Z¹, Z² and R^{a} are as defined herein.

In exemplary embodiments the conducting polymer comprises a monomer unit of formula (2C) or (2D):

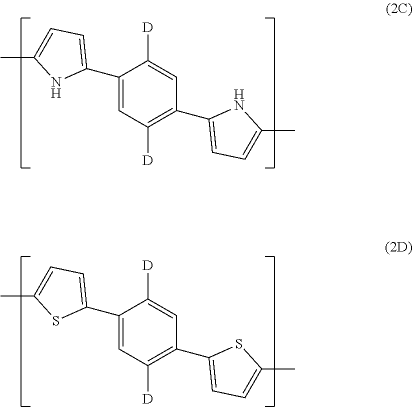

wherein D is as defined herein.

In some embodiments the conducting polymer further comprises at least one monomer unit different to the monomer unit of the formula (2).

In various embodiments the conducting polymer further comprises a monomer unit of formula (3), (4), (5), or a mixture of any two or more thereof:

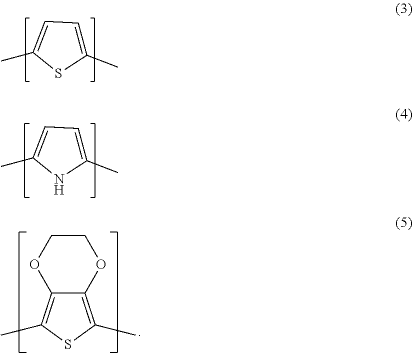

In various embodiments the conducting polymer further comprises a monomer unit of formula (6):

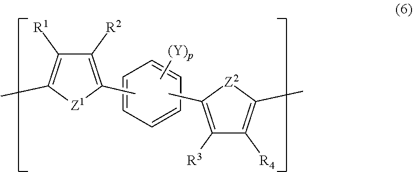

wherein

p, R¹, R², R³, R⁴, Z¹, and Z² are as defined herein; and

Y at each instance of p is independently selected from the group consisting of a water solubilising and/or protein repellent group, hydrogen, alkoxy, polyether, polyether alcohol, alkyl, alkenyl, cycloalkyl, cycloalkenyl, aryl, arylalkyl, heterocyclyl, and heteroaryl, wherein each alkyl, alkenyl, cycloalkyl, cycloalkenyl, aryl, arylalkyl, heterocyclyl, and heteroaryl is optionally substituted with one or more substituents independently selected from halo, nitro, nitrile, —C(O)R⁵, —OR⁵, —C(O)OR⁵, —OC(O)R⁵, —NR⁵R⁵, —C(O)NR⁵R⁵, —NR⁵C(O)R⁵, —NR⁵C(O)NR⁵R⁵, and alkyl.

In various embodiments, Y at each instance of p is independently selected from the group consisting of a water solubilising and/or protein repellent group, hydrogen, alkoxy, polyether, and polyether alcohol.

In various embodiments, Y at each instance of p is independently is selected from the group consisting of alkoxy, polyether, and polyether alcohol.

In various embodiments, Y at each instance of p is independently is selected from the group consisting of polyether and polyether alcohol.

In some embodiments Y at each instance of p is independently is selected from the group consisting of polyether.

In various embodiments, the polyether or polyether alcohol comprises from 2-50, 2-40, 2-30, 2-20, 2-10, 2-8, 2-6, or 2-4 monomer units.

In some embodiments the conducting polymer further comprises a monomer unit of formula (6A):

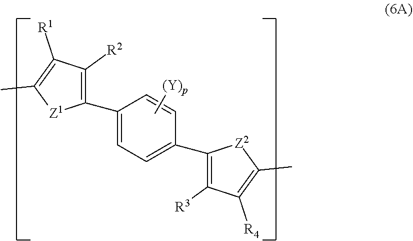

wherein

p, R¹, R², R³, R⁴, Z¹, Z² and Y are as defined herein.

In various embodiments the conducting polymer further comprises a monomer unit of formula (6B):

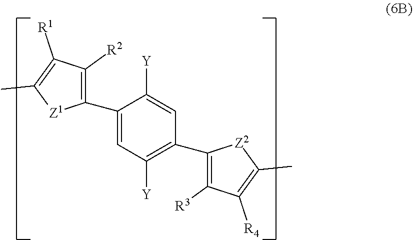

wherein

R¹, R², R³, R⁴, Z¹, Z² and Y are as defined herein.

In exemplary embodiments the conducting polymer as described herein further comprises a monomer unit of formula (6C) or (6D):

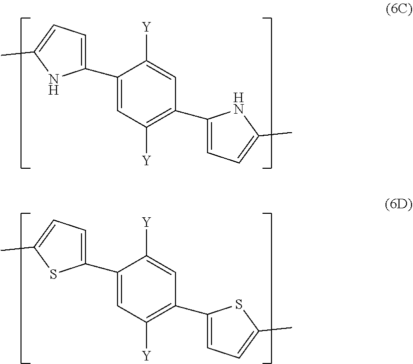

wherein Y is as defined herein.

In some embodiments each Y is identical.

In exemplary embodiments monomer unit of the formula (2) and monomer unit of the formula (6) are identical except for the D and Y groups.

In various embodiments, the ratio of the monomer unit of formula (2) to the at least one monomer unit different to the monomer unit of the formula (2), for example a monomer unit of the formula (3), (4) or (5), is from about 10:1 to 1:10,000, for example 10:1 to 1:1000, 10:1 to 1:100, 1:1 to 1:10,000, 1:1 to 1:1000, or 1:1 to 1:100.

In some embodiments the ratio of the monomer unit of formula (2) to the monomer unit of the formula (6) is from about 10:1 to 1:1,000, 10:1 to 1:500, 10:1 to 1:100, 1:1 to 1:100, 1:1 to 1:50, 1:1 to 1:5, or 1:2 to 1:4, or about 1:3.

In some embodiments the method comprises co-polymerising the polymerisable monomer of formula (1) and at least one additional polymerisable monomer different to the monomer of formula (1) to provide the conducting polymer.

In various embodiments the method comprises co-polymerising the polymerisable monomer of formula (1) and thiophene, pyrrole, 3,4-ethylenedioxythiophene (EDOT), or a mixture of any two or more thereof.

In exemplary embodiments the method of making a conducting polymer comprises co-polymerising the polymerisable monomer of formula (1) and a polymerisable monomer of formula (7):

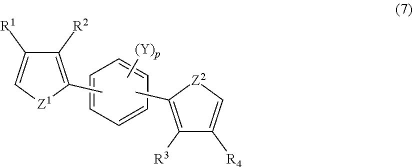

wherein p, R¹, R², R³, R⁴, Z¹, Z² and Y are as defined herein.

In some embodiments the method comprises co-polymerising the polymerisable monomer of formula (1) and a polymerisable monomer of formula (7A):

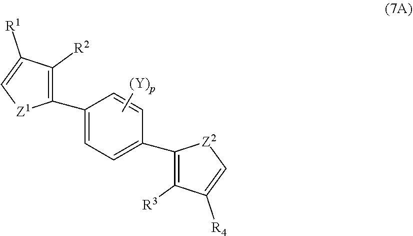

wherein p, R¹, R², R³, R⁴, Z¹, Z² and Y are as defined herein.

In some embodiments the method comprises co-polymerising the polymerisable monomer of formula (1) and a polymerisable monomer of formula (7B):

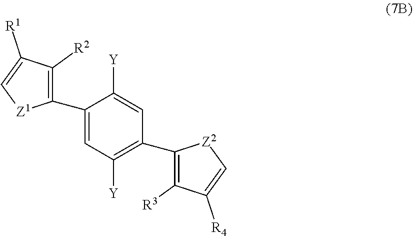

wherein R¹, R², R³, R⁴, Z¹, Z² and Y are as defined herein.

In some embodiments the method comprises co-polymerising the polymerisable monomer of formula (1) and a polymerisable monomer of formula (7C) or (7D):

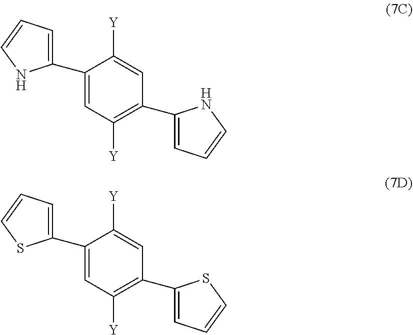

wherein Y is as defined herein.

In some embodiments the method comprises:

(i) providing a plurality of monomers of the formula (1) as defined herein

(ii) providing a substrate; and

(iii) polymerising each monomer of the formula (1) to provide a conducting polymer and depositing each conducting polymer at a separate, predetermined location on a surface of the substrate to provide a coating of the conducting polymer at the location; or

(iii) depositing each monomer of the formula (1) at a separate, predetermined location on a surface of the substrate and polymerising each monomer to provide a coating of the conducting polymer at the location;

wherein at least two locations on the surface of the substrate are coated with a conducting polymer having a different probe.

In some embodiments the method comprises:

(i) providing a plurality of monomers of the formula (1) as defined herein;

(ii) providing a substrate comprising a plurality of electrodes; and

(iii) polymerising each monomer of the formula (1) to provide a conducting polymer and depositing each conducting polymer on a surface of a different electrode to provide a coating of the conducting polymer on the surface of the electrode; or

(iii) depositing each monomer of the formula (1) on a surface of a different electrode and polymerising each monomer to provide a coating of the conducting polymer on the surface of the electrode;

wherein the surfaces of at least two of the electrodes are coated with a conducting polymer having a different probe.

Preferably, each conducting polymer coated on the surface of the substrate or electrode has a different probe. Preferably the different probes are adapted to bind or capable of binding different targets.

In some embodiments the monomer(s) are deposited on the surface of the substrate or electrode and polymerised to provide a coating of the conducting polymer on the surface of the substrate or electrode.

In some embodiments, the monomer(s) are polymerised by electroless oxidative polymerisation, wherein the oxidant is oxygen or hydrogen peroxide.

In various embodiments, the monomer(s) are polymerised by electroless oxidative polymerisation, wherein the oxidant is air or dissolved oxygen.

In some embodiments the electroless oxidative polymerisation is catalysed by an oxygen or hydrogen peroxide reduction catalyst.

In some embodiments the catalyst for the electroless oxidative polymerisation comprises Pt, Pd, Ru, or Ir; an oxide of Pt, Pd, Ru, or Ir; carbon (for example carbon nanotubes, fullerines, or graphene); or a mixture of any two or more thereof.

In various embodiments, the electroless oxidative polymerisation catalyst is Pt or Pd.

In various embodiments, the electroless oxidative polymerisation catalyst is Pt.

In various embodiments the electroless oxidative polymerisation catalyst is in the form of nano-particles.

In some embodiments the method comprises monomer(s) that are stable to oxidative polymerisation by oxygen or hydrogen peroxide in the absence of an oxygen or hydrogen peroxide reduction catalyst for at least 4, 8, 12, 24 or 48 hours.

In some embodiments the surface of the substrate or electrode on which the conducting polymer(s) or monomer(s) are deposited consists of or comprises the catalyst.

In various embodiments the electroless oxidative polymerisation is of monomer(s) wherein Z¹ and Z² are each S.

In some embodiments the oxidative polymerisation provides a polymer film having a thickness of from about 5 nm to 10 μm, preferably from 5 nm to 100 nm, for example from 5 nm to 75 nm, from 5 nm to 50 nm, from 5 nm to 25 nm, from 10 nm to 100 nm, from 10 nm to 75 nm, from 10 nm to 50 nm, from 10 nm to 25 nm, from 20 nm to 100 nm, from 20 nm to 75 nm, from 20 nm to 50 nm, from 20 nm to 25 nm, from 30 nm to 100 nm, from 30 nm to 75 nm, from 30 nm to 50 nm, from 40 nm to 100 nm, from 40 nm to 75 nm, from 40 nm to 50 nm, from 50 nm to 100 nm, or from 50 nm to 75 nm, when carried out for a period of time from about 1 second to about 120 seconds.

In some embodiments the monomer(s) are polymerised by electropolymerisation.

In some embodiments the electropolymerisation is carried out at a potential of about 0 to about 1.0V vs. Ag/AgCl (3 M KCl), for example from about 0.2 to 1.0, 0.3 to 1.0, 0.4 to 1.0, 0.5 to 1.0, 0.6 to 1.0, 0.7 to 1.0, 0.8 to 1.0, 0.2 to 0.9, 0.3 to 0.9, 0.4 to 0.9, 0.5 to 0.9, 0.6 to 0.9, 0.7 to 0.9, or 0.8 to 0.9V vs. Ag/AgCl (3 M KCl).

In some embodiments the electropolymerisation provides a polymer film having a thickness of from about 5 nm to 10 μm, preferably from 5 nm to 100 nm, when carried out for a period of time from about 0.1 seconds to about 10 seconds. In some embodiments the electropolymerisation provides a polymer film having a thickness of from about 5 nm to 10 μm, preferably from 5 nm to 100 nm, when carried out for a period of time from about 0.1 seconds to about 20 seconds, or from about 0.1 seconds to about 30 seconds.

In some embodiments the sensor comprises a substrate comprising at least one electrode having a surface coated with a conducting polymer as described herein.

In some embodiments the sensor comprises a substrate comprising a plurality of electrodes, each electrode comprising a surface coated with a conducting polymer as described herein, wherein the surfaces of at least two of the electrodes are coated with a conducting polymer having a different probe.

In various embodiments, the detector is capable of detecting binding of a target by a probe.

In some embodiments the sensor system comprises a detector capable of measuring an electrochemical property of the conducting polymer.

In some embodiments the sensor system comprises a detector capable of measuring the impedance of the conducting polymer.

In some embodiments the sensor or sensor system further comprises a redox couple.

In some embodiments the sensor or sensor system comprises a counter electrode and optionally a reference electrode.

In some embodiments the sensor system may comprise a positive control. For example in some embodiments the system may comprise a positive control sample comprising a target, which probes of the conducting polymer(s) are capable of binding.

In some embodiments of the method, sensor, or sensor system as described herein, the electrode(s) on which the conducting polymer(s) is/are coated is/are a gold (e.g. screen printed gold), platinum, carbon (e.g. glassy or screen printed carbon), stainless steel, indium tin oxide (ITO), or doped silicon wafer electrode.

In certain embodiments of the method, sensor or sensor system as described herein, the electrode(s) on which the conducting polymer(s) are coated is a screen printed carbon electrode.

In some embodiments, the electrode(s) on which the conducting polymer(s) are coated is a screen printed electrode, such as screen printed carbon electrode, the surface of which has been modified prior to formation of the coating of the conducting polymer(s) by a treatment that increases the sensitivity of the electrode to detection of the target.

In some embodiments, the treatment is selected from laser glazing or plasma treatment.

In some embodiments of the method for determining the presence or absence or amount of a target in a sample comprises detecting binding of the target when present in the sample by a probe.

In some embodiments, the presence or absence or amount of a target in a sample is determined electrochemically or the presence or absence or amount of a target in a sample is detected electrochemically.

In some embodiments, the presence or absence or amount of a target in a sample is determined by electrochemical impedance spectroscopy or the presence or absence or amount of a target in a sample is detected by electrochemical impedance spectroscopy.

In some embodiments the method comprises contacting the sample and the sensor in the presence of a redox couple.

In various embodiments the redox couple is ferro-ferricyanide.

In some embodiments the method comprises amplifying a target nucleic acid in a sample according to the eighth aspect.

In various embodiments, the sample comprises double stranded nucleic acid.

In various embodiments, the sample comprises genomic nucleic acid.

In some embodiments, the sample comprises a lysate.

In various embodiments, the sample comprises a lysate comprising genomic nucleic acid.

In various embodiments, the lysate is a cell lysate.

In some embodiments, the cell lysate is a bacterial cell lysate.

In various embodiments, the sample or lysate comprises nucleic acid, preferably genomic nucleic acid, protein, lipids and other components, for example cellular components, produced by lysis.

In various embodiments, the sample comprises a lysate from which at least a portion of solid components or particles produced by lysis have been removed.

In various embodiments, the sample has not been subjected to nucleic acid extraction and/or purification.

In various embodiments, the sample has not been subjected to a nucleic acid extraction and/or purification comprising treatment with a proteinase, treatment (for example extraction) with one or more organic solvents, precipitation of the nucleic acid, and/or purification and/or isolation of the precipitated nucleic acid.

In various embodiments, the sample has not been subjected to a nucleic acid extraction and/or purification comprising treatment with one or more organic solvents.

In some embodiments the reaction mixture comprises a second single-stranded nucleic acid molecule comprising a nucleic acid sequence complementary to a second portion of the target nucleic acid sequence.

In some embodiments the reaction mixture comprises the first single-stranded nucleic acid molecule, or a single-stranded nucleic acid molecule capable of hybridizing to the first portion of the target nucleic acid sequence.

In some embodiments the method comprises the additional step of determining the presence or amount of polynucleotide in the reaction volume on the basis of the one or more impedance measurements.

In some embodiments the method comprises the additional step of measuring the impedance of the first electrode before the first elongation step of the nucleic acid amplification reaction.

In some embodiments the impedance is measured continuously throughout at least a portion of the polymerase chain reaction.

In some embodiments the method comprises measuring cumulative charge passed through the electrode.

In some embodiments the method comprises measuring cumulative charge passed through the electrode and terminating the polymerisation on the basis of the measurement.

In some embodiments the method comprises measuring cumulative charge passed through the electrode and terminating the polymerisation when a total charge of from about 1.0×10⁻⁵ C to about 5×10⁻⁵ C is measured.

In some embodiments the redox couple is a ferro-ferricyanide.

In some embodiments the target nucleic acid is present at an initial concentration of less than 1 pg/mL.

In some embodiments the target nucleic acid is present at an initial concentration of less than 1 fg/mL.

In some embodiments, the apparatus additionally comprises a thermocycler suitable for PCR.

In some embodiments, the apparatus additionally comprises a device for measuring the impedance of at least the first electrode.

In some embodiments, the device for measuring impedance is an LCR meter or is a potentiostat.

In some embodiments the device for measuring impedance is an LCR meter, a potentiostat, or the device measures impedance by determining the transconductance of or at the first electrode or by cyclic voltammetry.

In various embodiments, the sample comprises a double stranded nucleic acid and the method comprises:

heating the sample for a period at a temperature sufficient to dissociate the nucleic acid strands, and

contacting the dissociated nucleic acid strands with the sensor or sensor system of the present invention, and

cooling to anneal the target nucleic acid with a probe of the sensor or sensor system.

In various embodiments, the sample comprises microbes (for example, cells, such as bacteria, or viruses) comprising a target nucleic acid and the method comprises: lysing the microbes,

heating the sample for a period at a temperature sufficient to dissociate double stranded nucleic acid contained therein,

contacting the dissociated nucleic acid strands with the sensor or sensor system of the present invention, and

cooling to anneal the target nucleic acid with a probe of the sensor or sensor system.

In various embodiments, the sample comprises microbes (for example, cells, such as bacteria, or viruses) comprising a target nucleic acid and the method comprises: heating the sample for a period at a temperature sufficient to lyse the microbes and dissociate double stranded nucleic acid contained therein,

contacting the dissociated nucleic acid strands with the sensor or sensor system of the present invention, and

cooling to anneal the target nucleic acid with a probe of the sensor or sensor system.

In various embodiments, the method is for determining the presence or absence or amount of a target nucleic acid in an aqueous sample which may comprise a double stranded nucleic acid (for example, double stranded DNA) and the method comprises: admixing into the sample a buffer (eg phosphate-buffered saline) and a redox couple (for example, potassium ferri- and ferro-cyanide),

contacting the resultant mixture with a sensor or sensor system of the present invention,

heating to about the melting temperature of the double stranded nucleic acid to dissociate the strands (for example to about 95° C. for 1-5 minutes),

cooling to a temperature at which the nucleic acid strands re-anneal (for example, about 40 to 50° C.) to anneal the target nucleic acid with a probe of the sensor or sensor system, and

detecting binding of the target by the probe (for example, by measuring a sensor signal (for example, impedance of the conducting polymer) over time).

In some embodiments, if the target is present the sensor signal increases over time (depending on the target concentration).

In some of such embodiments, the method is for determining the presence or absence or amount of a target nucleic acid in an aqueous sample which may comprise double stranded nucleic acid (for example, double stranded DNA) and the method comprises:

admixing into the sample a buffer (eg phosphate-buffered saline), a redox couple (for example, potassium ferri- and ferro-cyanide), and nucleotides, nucleic acids and enzymes for a nucleic acid amplification reaction (for example, polymerase amplification),

contacting the resultant mixture with a sensor or sensor system of the present invention,

oscillating the temperature so as to cause successively melting of the double stranded nucleic acid, amplification of the target nucleic acid, and annealing of the target nucleic acid to a probe of the sensor or sensor system, and

detecting binding of the target by the probe (for example, by measuring a sensor signal (for example, impedance of the conducting polymer) over time).

In some embodiments, if the target is present the sensor signal increases over time as the temperature is oscillated.

In various embodiments, the method is for determining the presence or absence or amount of bacteria comprising a target nucleic acid in a water sample which may comprise the bacteria, and the method comprises:

admixing into the sample a buffer (eg phosphate-buffered saline) and a redox couple (for example, potassium ferri- and ferro-cyanide),

heating to lyse the bacteria (for example to about 95° C. for 5 min) and dissociate double stranded nucleic acid contained therein,

contacting the hot lysate with a sensor or sensor system of the invention,

cooling to a temperature at which the nucleic acid strands re-anneal (for example 40 to 50° C.) to anneal the target nucleic acid with a probe of the sensor or sensor system, and

detecting binding of the target by the probe (for example, by measuring a sensor signal (for example, impedance of the conducting polymer) over time).

In some embodiments, if the bacteria are present, the sensor signal increases with time (depending on the bacterial concentration).

In various embodiments, the method further comprises filtering or otherwise removing solid particles from the hot lysate.

In some of such embodiments, the method is for determining the presence or absence or amount of bacteria comprising a target nucleic acid in a water sample which may comprise the bacteria, and the method comprises:

admixing into the sample a buffer (eg phosphate-buffered saline), a redox couple (for example, potassium ferri- and ferro-cyanide), and nucleotides, nucleic acids and enzymes for a nucleic acid amplification reaction (for example, polymerase amplification),

heating to lyse the bacteria (for example to about 95° C. for 5 min) and dissociate double stranded nucleic acid contained therein,

contacting the hot lysate with a sensor or sensor system of the invention,

oscillating the temperature so as to cause successively melting of the double stranded nucleic acid, amplification of the target nucleic acid, and annealing of the target nucleic acid to a probe of the sensor or sensor system, and

detecting binding of the target by the probe (for example, by measuring a sensor signal (for example, impedance of the conducting polymer) over time).

In various embodiments, if the bacteria are present is present the sensor signal increases over time as the temperature is oscillated.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Although the present invention is broadly as defined above, those persons skilled in the art will appreciate that the invention is not limited thereto and that the invention also includes embodiments of which the following description gives examples.

### BRIEF DESCRIPTION OF THE FIGURES

The invention will be described with reference to the accompanying figures, in which:

FIG. 1 is a graph showing EIS measurements in the presence of 5 mM K₃Fe(CN)₆ and 5 mM K₄Fe(CN)₆. The EIS measurements were carried out after deposition of the homopolymer-oligonucleotide complex (homopolymer of monomer 22 with a DNA-probe bound) on the Pt disk electrode (CP and DNA), and after 1000nM target oligonucleotide hybridization to the homopolymer-DNA probe (1000nM target). The un-shaded squares represent experimental EIS values for the ferro-ferricyanide redox reaction on the homopolymer-oligonculeotide probe complex on the Pt disk electrode. The shaded diamonds represent experimental EIS values after hybridisation of the target oligonucleotide to the homopolymer-oligonucleotide probe. The data were fitted to a Randle's equivalent circuit (inset), consisting of solution resistance Ri, a constant phase element Q₂, a charge transfer resistance R₂ and a Warburg diffusion element (W₂) as indicated by the solid line. Changes in the parameters of the fitted model were used as signals for the detection of the target oligonucleotide.

FIG. 2 is a graph showing sensor response (EIS for the ferri-ferrocyanide redox reaction on the electrode surface) in the absence (BARE) and presence (CP & DNA) of different target concentrations (100 nM, 200 nM, 2 μM, 5 μM, 10 μM and 20 μM). The data were fitted to an equivalent circuit model (inset), consisting of solution resistance R₁, a constant phase element Q₂, a charge transfer resistance R₂ and a Warburg diffusion element (W₂) as indicated by the solid line, and changes in a parameter of the fitted model (charged transfer resistance) were used as signals for the detection of the target oligonucleotide.

FIG. 3 shows FTIR spectra before (I) and after (II) attachment of oligonucleotides (ON) onto A) monomer 38 and B) monomer 22.

FIG. 4 shows potentiodynamic electrocopolymerisation of A) pyrrole and monomer 50 to form P70 (Py: monomer 50 (50:1 mol/mol)) on a glassy carbon (GC) electrode (3 mm), B) monomer 7 and monomer 60 to form P80 (TGThP 7 : ThPhON 60 50:1 mol/mol) on a Au electrode (1.6 mm). The electopolymerisation was carried out for 5 cycles at scan rate of 100 mV/s in 1:1 PBS/DMF for P80 and 9:1 PBS/DMF for P70.

FIG. 5 shows the current-time trace, following application of a constant potential of +0.8V for electropolymerisation to form copolymers A) PyPhON-co-Py P70 (1:50 mole ratio) on a 3mm diameter GC electrode and B) ThPhON-co-ThPhEG P80 (1:50 mole ratio) on a 1.6mm diameter Au electrode. Prior to electrode deposition, Non-Hodgkin probe sequence was attached to the monomer 38 and PBGD sequence was attached to monomer 22. Electropolymerisation was carried out vs. Ag/AgCl for the GC electrode and vs. leak free reference for the gold electrode.

FIG. 6 shows cyclic voltammograms (CVs) of A) P70 and C) P80 in PBS buffer (pH 7.4) at various scan rates (100, 200, 300, 400 and 500 mV s⁻¹). The insets show Log of oxidation peak currents (y axis) vs. Log of scan rate (x axis). FIGS. 6B and 6D show SEM images of: B) P70 and D) P80.

FIG. 7 shows Nyquist plots for A) P(PyPhON-co-Py) P70 and B) P(ThPhON-co-ThPhEG) P80 upon hybridization with 1 μM and 1 nM target concentrations, respectively. Spectra after 10, 30, 60 and 90 minutes of incubation are shown.

FIG. 8 shows Nyquist diagrams of A) P70 and C) P80 electrodes after incubation with increasing concentrations of Non-Hodgkin and PBGB sequence solutions, respectively. Experimental data are presented as symbols and the fitting curves to the equivalent circuit as solid lines. The data were fitted to a Randle's equivalent circuit (inset), consisting of solution resistance Rs, a constant phase element CPE, a charge transfer resistance R_{CT} and a Warburg diffusion element (W) Normalized sensor responses, ΔR_{CT}/R_{CT}⁰, of the electrodes modified with B) P70 and D) P80 are shown versus the logarithm of the target concentration. Each experiment was repeated three times (n=3).

FIG. 9 shows the normalized sensor responses of A) P91 (poly(PyPhON-co-Py), polymer formed from the attachment of the Non-Hodgkin probe to monomer 38 and co-polymerising with pyrrole) and B) P92 (poly(ThPhON-co-ThPhEG), polymer formed from the attachment of the Non-Hodgkin probe to monomer 22 and copolymerising with monomer 7), sensing films upon incubation with non-complementary (Un-comp), a first base mismatched (1-mis; Non-Hodgkin mismatch A of Table 3), a second base mismatched (2-mis; Non-Hodgkin mismatch B of Table 3), and fully complementary (Comp) sequences. P91 electrodes were incubated with 1 μM of the oligonucleotide solutions, and P92 were incubated with 1 nM of the oligonucleotide solutions.

FIG. 10 shows the EIS spectra of electrochemically deposited films of A) polymer P63 (labelled ‘electrode 2’), B) polymer P64 (labelled ‘electrode 1’) and C) polymer P65 (labelled ‘electrode 3’) deposited on different Au electrodes 1-3 respectively, before (empty symbols) and after (solid symbols) incubation of the sensing films with a PBS solution containing two target oligonucleotides (Non-Hodgkin and PBGD genes) at concentrations of 1 μM. Polymer P64 carries a Non-Hodgkin lymphoma (Non-Hodgkin) probe, polymer P63 a chronic lymphocytic leukemia (PBGD) probe and polymer P65 a bladder cancer (FGFR3) probe.

FIG. 11A shows the cyclic voltammogram (CV) traces in PBS solution, at pH 7.4, of the GC electrodes in the presence of 5 mM each of ferri- and ferrocyanide, before (BARE GC, dotted trace) and after (PtNP modified GC, dashed trace) Pt nanoparticle (PtNP) deposition. Optical pictures of GC electrodes before and after Pt nanoparticle deposition (50× lens, Leica optical microscopy) are shown in FIGS. 11B and 11C respectively.

FIG. 12A shows the CV trace for the ferro-ferricyanide redox reaction in PBS solution at pH 7.4 before (GC-PtNP, dashed trace) and after (CP deposited GC-PtNP, dotted trace) deposition of conducting polymer (CP) from a solution containing monomer 22 and monomer 7, mole ratio 1:50, in PBS only. FIG. 12B shows how the EIS spectrum for the ferro-ferricyanide redox changes before (GC-PtNP, dashed trace) and after (CP deposited GC-PtNP, dotted trace) co-polymerisation of monomer 22 and monomer 7. FIG. 12C shows an optical picture of GC electrodes after deposition of the co-polymer of monomers 22 and 7 (50× lens. Leica optical microscopy).

FIG. 13A shows the CV trace for the ferro-ferricyanide redox reaction in PBS solution at pH 7.4 before (GC-PtNP, dashed trace) and after (CP deposited GC-PtNP, dotted trace) deposition of conducting polymer from a solution containing monomer 22 and monomer 7 (mole ratio 1:50) in PBS also containing 0.1M sodium tosylate (NaTos). EIS of the GC electrodes with the Pt nanoparticles in PBS solution, pH 7.4, containing 5 mM each of ferri- and ferrocyanide before (GC-PtNP, dashed trace) and after (CP deposited GC-PtNP, dotted trace) deposition of the co-polymer of monomers 22 and 7 in the presence of NaTos (to accelerate polymer formation) as dopant are shown in FIG. 13B. FIG. 13C shows optical pictures of GC electrodes after deposition of co-polymer of monomers 22 and 7 (5× lens. Leica optical microscopy). Inset is 50x lens.

FIG. 14A shows the CV traces of a Pt nanoparticle-activated GC electrode in PBS buffer, at pH 7.4 before exposure (dotted trace), and after 30, 60 and 120 seconds (black trace) of exposure of the electrode to a solution of monomer 60 and monomer 7 (mole ratio 1:50) in PBS solution containing 0.1M NaTos. Microscopic pictures of the Pt nanoparticle-activated GC electrode before exposure, 14B), and of P80 (poly(ThPhCOOH-co-ThPhEG)) deposited electrode after 14C) 30 seconds, 14D) 60 second, 14E) 120 second, 14F) 240 seconds, 14G) 360 seconds of exposure to the mixed monomer solution (50× Lens).

FIG. 15 shows the relative change in charge transfer resistance for the redox reaction of ferro/ferricyanide on the conducting polymer film, measured in PBS, pH 7.4, containing 5 mM of each of K₃Fe(CN)₆ and K₄Fe(CN)₆, on a Pt nanoparticle-activated GC electrode after increasing time of exposure to the mixed solution of monomer 60 and monomer 7 in PBS/NaTos followed by washing. The change in charge transfer resistance is expressed with respect to the charge transfer resistance measured in the ferro-ferricyanide solution before exposure of the electrode to the mixed monomer solution.

FIGS. 16A and 18A are Nyquist plots that show the response of electrodes comprising P100 (P(PyPhON-Py)) and P200 (P(PyPhON-PyPhEG)) sensing films respectively to different concentrations of synthetic E. coli target DNA. The Nyquist plot corresponding to the probe only (indicated by squares) represents the response observed in the absence of ssON synthetic E. coli F1630 target DNA (5′ CCTTCCTAGCTGTCTAAACTAG 3′ (SEQ ID NO: 10)). For graph 16A the data points plotted as circles, triangles, diamonds and stars correspond to the electrode responses at 100 aM, 1 fM, 10 fM and 100 fM of the synthetic E. coli target DNA respectively. For graph 18A the data points plotted as triangles, diamonds, circles and stars correspond to the electrode responses at 100 aM, 1 fM, 10 fM and 100 fM of ssON synthetic E. coli F1630 target DNA respectively.

FIGS. 16B and 18B show the response of electrodes comprising P100 (P(PyPhON-Py)) and P200 (P(PyPhON-PyPhEG)) sensing films respectively to different concentrations of ssON synthetic E. coli F1630 target DNA (5′ CCTTCCTAGCTGTCTAAACTAG 3′ (SEQ ID NO: 10)).

FIGS. 16C and 18C compare the response of electrodes comprising P100 (P(PyPhON-Py)) sensing films (Comp) with electrodes comprising P300 sensing films (Non-Comp) (FIG. 16C), and electrodes comprising P200 (P(PyPhON-PyPhEG)) (Comp.) sensing films and electrodes comprising P400 (Non-Comp) sensing films (FIG. 18C) respectively to ssON synthetic E. coli F1630 target DNA (5′ CCTTCCTAGCTGTCTAAACTAG 3′ (SEQ ID NO: 10)) present at a concentration of 10 fM.

FIGS. 17A and 19A are Nyquist plots that show the response of electrodes comprising P100 (P(PyPhON-Py)) and P200 (P(PyPhON-PyPhEG)) sensing films respectively to different concentrations of extracted genomic E. coli BL21 target DNA. The Nyquist plot corresponding to the probe only (indicated by squares) represents the response observed in the absence of extracted genomic E. coli BL21 target DNA For graph 17A the data points plotted as circles, triangles, diamonds and stars correspond to the electrode responses at 100 aM, 1 fM, 10 fM and 100 fM of extracted genomic E. coli BL21 target DNA respectively. For graph 19A the data points plotted as circles, triangles, diamonds, and stars correspond to the electrode responses at 100 aM, 1 fM, 10 fM and 100 fM of extracted genomic E. coli BL21 target DNA respectively.

FIGS. 17B and 19B show the response of electrodes comprising P100 (P(PyPhON-Py)) and P200 (P(PyPhON-PyPhEG)) sensing films respectively to different concentrations of extracted genomic E. coli BL21 target DNA.

FIGS. 17C and 19C compare the response of electrodes comprising P100 (P(PyPhON-Py)) sensing films (Comp) with electrodes comprising P300 sensing films (Non-Comp) (FIG. 17C), and electrodes comprising P200 (P(PyPhON-PyPhEG)) (Complementary) sensing films and electrodes comprising P400 (Uncomplementary) sensing films (FIG. 19C) respectively to extracted genomic E. coli BL21 target DNA present at a concentration of 10 fM.

FIG. 20A is a Nyquist plot that shows the response of an electrode comprising a P200 (P(PyPhON-PyPhEG)) sensing film to different concentrations of crude E. coli BL21 lysate DNA. The Nyquist plot corresponding to the probe only (indicated by squares) represents the response observed in the absence of E. coli BL21 lysate DNA.

FIG. 20B shows the response of an electrode comprising a P200 (P(PyPhON-PyPhEG)) sensing film to different concentrations of crude E. coli BL21 lysate DNA.

FIG. 20C compares the response of electrodes comprising P200 (P(PyPhON-PyPhEG)) (Comp.) sensing films and electrodes comprising P400 (Non-Comp) sensing films to crude E. coli BL21 lysate DNA present at a concentration of 10 fM.

FIG. 21 shows the continuous kinetics measurements of the ssON synthetic E. coli F1630 target DNA (5′ CCTTCCTAGCTGTCTAAACTAG 3′ (SEQ ID NO: 10)) binding experiments performed in the presence of the 5 mM [Fe (CN)₆^{3-/4-}] with and without stirring the solution comprising the synthetic E. coli target DNA.The grey bars and the black bars represent 10 fM of synthetic E. coli target DNA binding to P200 P(PyPhON-PyPhEG) without mixing (10 fM without mix) the solution and with constantly mixing at 50 rpm (10 fM with mix) respectively.

FIG. 22 compares the response of sensors based on electrodes comprising P100 (PyPhON-Py) sensing films and P200 (PyPhON-PyPhEG) sensing films to ssON synthetic E. coli F1630 target DNA (5′ CCTTCCTAGCTGTCTAAACTAG 3′ (SEQ ID NO: 10)) (10 fM).

FIG. 23 compares the response of sensors based on electrodes comprising P100 (P(PyPhON-Py)) sensing films and P200 (P(PyPhON-PyPhEG)) sensing films to extracted genomic E. coli BL21 DNA (10 fM) respectively.

FIG. 24 compares the response of a sensor based on P200 (P(PyPhON-PyPhEG)) in the presence of ssON synthetic E. coli F1630 target DNA (5′ CCTTCCTAGCTGTCTAAACTAG 3′ (SEQ ID NO: 10)) (synthetic), extracted E. coli genomic BL21 DNA samples (extracted) and crude E. coli BL21 lysate DNA (crude bacterial) (10 fM).

FIG. 25 shows the increase in impedance of a screen-printed carbon electrode functionalized with P200 P(PyPhON-PyPhEG) at different concentrations of ssON synthetic E. coli F1630 target DNA (5′ CCTTCCTAGCTGTCTAAACTAG 3′ (SEQ ID NO: 10)) sequence, where the squares correspond to impedance measurements of the functionalised electrode after 20 seconds in the absence of target DNA and upright triangles ( [symbol] ), sideways triangles ( [symbol] ), diamonds and circles correspond to the impedance of the electrode after incubation with 1 fM, 100 fM, 10 pM and 100 pM concentrations of target DNA respectively.

FIG. 26 compares dose-response for screen printed carbon P200 (poly(PyPhON-PyPhEG) modified Gwent electrodes formed by different electropolymerisation times (5 s, 7 s, 10 s, 15 s and 20 s represented by squares, circles, triangles pointing up ( [symbol] ), triangles pointing down ( [symbol] ) and triangles pointing to the left ( [symbol] ) respectively) after incubation in PBS buffer with 1 fM, 100 fM and 10 pM ssON synthetic E. coli F1630 target DNA (5′ CCTTCCTAGCTGTCTAAACTAG 3′ (SEQ ID NO: 10)).

### DETAILED DESCRIPTION OF THE INVENTION

Definitions

The term “comprising” as used in this specification and claims means “consisting at least in part of”. When interpreting each statement in this specification and claims that includes the term “comprising”, features other than that or those prefaced by the term may also be present. Related terms such as “comprise” and “comprises” are to be interpreted in the same manner.

As used herein the term “and/or” means “and” or “or”, or both.

As used herein “(s)” following a noun means the plural and/or singular forms of the noun.

The general chemical terms used in the formulae herein have their usual meanings.

Unless indicated otherwise, nomenclature used to describe chemical groups or moieties as used herein follow the convention where, reading the name from left to right, the point of attachment to the rest of the molecule is at the right-hand side of the name. For example, the group “arylalkyl” is attached to the rest of the molecule at the alkyl end.

The term “alkyl” employed alone or in combination with other terms, unless indicated otherwise, refers to a straight chain or branched chain hydrocarbon group having from 1 to 12 carbon atoms. In some embodiments, alkyl groups have from 1 to 10, from 1 to 8, from 1 to 6, or from 1 to 4 carbon atoms. Examples of straight chain alkyl groups include, but are not limited to, methyl, ethyl, n-propyl, n-butyl, n-pentyl, n-hexyl, n-heptyl, and n-octyl. Examples of branched alkyl groups include, but are not limited to, isopropyl, iso-butyl, sec-butyl, tert-butyl, neopentyl, isopentyl, and 2,2-dimethylpropyl.

The term “alkenyl” employed alone or in combination with other terms, unless indicated otherwise, refers to a straight or branched chain hydrocarbon group having from 2 to 12 carbon atoms and having at least one double bond between two carbon atoms. In some embodiments, alkenyl groups have from 2 to 10, from 2 to 8, from 2 to 6, or from 2 to 4 carbon atoms. In some embodiments, alkenyl groups have one, two, or three carbon-carbon double bonds. Examples of alkenyl groups include, but are not limited to, vinyl, allyl, —CH═CH(CH₃), —CH═C(CH₃)₂, —C(CH₃)═CH₂, and —C(CH₃)═CH(CH₃).

The term “cycloalkyl” employed alone or in combination with other terms, unless indicated otherwise, refers to a mono-, bi- or tricyclic hydrocarbon group having from 3 to 12 carbon atoms in the ring(s). In some embodiments, cycloalkyl groups have from 3 to 10, from 3 to 8, from 3 to 7, from 3 to 6, from 4 to 6, from 3 to 5 or from 4 to 5 carbon atoms in the ring(s). In some embodiments, cycloalkyl groups have 5 or 6 ring carbon atoms. Examples of monocyclic cycloalkyl groups include, but are not limited to, cyclopropyl, cyclobutyl, cyclopentyl, cyclohexyl, cycloheptyl, and cyclooctyl. Bi- and tricyclic ring systems include bridged, spiro, and fused cycloalkyl ring systems. Examples of bi- and tricyclic ring cycloalkyl systems include, but are not limited to, bicyclo[2.1.1]hexanyl, bicyclo[2.2.1]heptanyl, adamantyl, and decalinyl.

The term “cycloalkenyl” employed alone or in combination with other terms, unless indicated otherwise, refers to a non-aromatic mono-, bi- or tricyclic hydrocarbon groups having from 4 to 12 carbon atoms in the ring(s) and having at least one double bond between two carbon atoms. In some embodiments, cycloalkenyl groups have one, two or three double bonds. In some embodiments, cycloalkenyl groups have from 5 to 12, from 5 to 10, from 5 to 8, or from 5 to 6 carbon atoms in the ring(s). In some embodiments, cycloalkenyl groups have 5, 6, 7, or 8 ring carbon atoms in the ring(s). Examples of cycloalkenyl groups include cyclohexenyl, cyclopentenyl, cyclohexadienyl, butadienyl, pentadienyl, and hexadienyl.

The term “aryl” employed alone or in combination with other terms, unless indicated otherwise, refers to a cyclic aromatic hydrocarbon group having from 6 to 14 carbon atoms in the ring(s) and no heteroatoms in the ring(s). Aryl groups include monocyclic, fused bicyclic, and fused tricyclic ring systems. Examples of aryl groups include, but are not limited to, phenyl, azulenyl, heptalenyl, fluorenyl, phenanthrenyl, anthracenyl, indenyl, indanyl, pentalenyl, and naphthyl. In some embodiments, aryl groups have from 6 to 12, or from 6-10 carbon atoms in the ring(s). In some embodiments, the aryl groups are phenyl or naphthyl. Aryl groups include aromatic-aliphatic fused ring systems. Examples include, but are not limited to, indanyl and tetrahydronaphthyl.

The term “heterocyclyl” employed alone or in combination with other terms, unless indicated otherwise, refers to a non-aromatic ring system containing from 3 to 16 atoms in the ring(s), of which one or more is a heteroatom. In some embodiments, the heteroatom is nitrogen, oxygen, or sulfur. In some embodiments, the heterocyclyl group contains one, two, three, or four heteroatoms. In some embodiments, heterocyclyl groups include mono-, bi- and tricyclic rings having from 3 to 16, from 3 to 14, from 3 to 12, from 3 to 10, from 3 to 8, or from 3 to 6 atoms in the ring(s). Heterocyclyl groups include partially unsaturated and saturated ring systems, for example, imidazolinyl and imidazolidinyl. Heterocyclyl groups include fused and bridged ring systems containing a heteroatom, for example, quinuclidyl. Heterocyclyl groups include, but are not limited to, aziridinyl, azetidinyl, azepanyl, diazepanyl, 1,3-dioxanyl, 1,3-dioxolanyl, isoxazolidinyl, morpholinyl, piperazinyl, piperidinyl, pyranyl, pyrazolidinyl, pyrrolinyl, pyrrolidinyl, tetrahydrofuranyl, tetrahydrothienyl, thiadiazolidinyl, and trithianyl. In some embodiments, heterocyclyl groups have 5 or 6 ring carbon atoms.

The term “heteroaryl” employed alone or in combination with other terms, unless indicated otherwise, refers to an aromatic ring system containing from 5 to 16 atoms in the ring(s) and at least one heteroatom in the ring(s). In some embodiments, the heteroatom is nitrogen, oxygen, sulfur, or selenium, preferably oxygen, nitrogen, or sulfur. In some embodiments, heteroaryl groups comprise 1, 2, or 3 heteroatoms in the ring(s). In some embodiments, heteroaryl groups include monocyclic, fused bicyclic, and fused tricyclic ring systems having from 5 to 16, from 5 to 14, from 5 to 12, from 5 to 10, from 5 to 8, or from 5 to 6 atoms in the ring(s). Heteroaryl groups include, but are not limited to, pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, selenophenyl, benzothiophenyl, furanyl, benzofuranyl, indolyl, azaindolyl (pyrrolopyridinyl), indazolyl, benzimidazolyl, pyrazolopyridinyl, triazolopyridinyl, benzotriazolyl, benzoxazolyl, benzothiazolyl, imidazopyridinyl, isoxazolopyridinylxanthinyl, guaninyl, quinolinyl, isoquinolinyl, tetrahydroquinolinyl, quinoxalinyl, and quinazolinyl. Heteroaryl groups include fused ring systems in which all of the rings are aromatic, for example, indolyl, and fused ring systems in which only one of the rings is aromatic, for example, 2,3-dihydroindolyl.The term “halo” or “halogen” employed alone or in combination with other terms is intended to include F, Cl, Br, and I.

As used herein, the term “substituted” is intended to mean that one or more hydrogen atoms in the group indicated is replaced with one or more independently selected suitable substituents, provided that the normal valency of each atom to which the substituent/s are attached is not exceeded, and that the substitution results in a stable compound.

The term “stable” as used herein refers to compounds which possess stability sufficient to allow manufacture and which maintain their integrity for a period of time sufficient to be useful for the purposes described herein.

The term “electron withdrawing group” is intended to mean an atom or a functional group that removes electron density from a conjugated or aromatic ring system via resonance or inductive effects, for example a nitro group.

The term “electron donating group” is intended to mean an atom or a functional group that donates electron density into a conjugated or aromatic ring system via resonance or inductive effects, for example an alkoxy group.

The term “polyether” as used herein refers to a group of formula —O—(C₁₋₆alkyl-O)_{q}—C₁₋₆alkyl, wherein q is an integer from 2-50. For example, q may be an integer from 2-40, from 2-30, from 2-20, from 2-10, from 2-9, from 2-8, from 2-7, from 2-6, from 2-5, from 2-4, from 3-50, from 3-40, from 3-30, from 3-20, from 3-10, from 3-9, from 3-8, from 3-7, from 3-6, from 3-5, or from 3-4.

The term, “polyether alcohol” as used herein refers to a group of formula —O—(C₁₋₆alkyl-O)_{q}—H, wherein q is an integer from 2-50. For example, q may be an integer from 2-40, from 2-30, from 2-20, from 2-10, from 2-9, from 2-8, from 2-7, from 2-6, from 2-5, from 2-4, from 3-50, from 3-40, from 3-30, from 3-20, from 3-10, from 3-9, from 3-8, from 3-7, from 3-6, from 3-5, or from 3-4.

Asymmetric centers may exist in the compounds described herein. Asymmetric centers may be designated as (R) or (S), depending on the configuration of substituents in three dimensional space at the chiral atom. All stereochemical isomeric forms of the compounds, including diastereomeric, enantiomeric, and epimeric forms, as well as d-isomers and I-isomers, and mixtures thereof, including enantiomerically enriched and diastereomerically enriched mixtures of stereochemical isomers, are within the scope of the invention.

The compounds described herein may also exist as conformational or geometric stereoisomers, including cis, trans, syn, anti, entgegen (E), and zusammen (Z) isomers. All such stereoisomers and mixtures thereof are within the scope of the invention.

Also within the scope of the invention are any tautomeric isomers or mixtures thereof of the compounds described. As would be appreciated by those skilled in the art, a wide variety of functional groups and other structures may exhibit tautomerism. Examples include, but are not limited to, keto/enol and imine/enamine tautomerism.

Also within the scope of the invention are salts of the compounds described herein. Such salts include, acid addition salts, base addition salts, and quaternary salts of basic nitrogen-containing groups. Acid addition salts can be prepared by reacting compounds, in free base form, with inorganic or organic acids. Base addition salts can be prepared by reacting compounds, in free acid form, with inorganic or organic bases. Quaternary salts of basic nitrogen-containing groups in the compounds may be may be prepared by, for example, reaction with alkyl halides.

The compounds described herein may form or exist as solvates with various solvents. If the solvent is water, the solvate may be referred to as a hydrate, for example, a mono-hydrate, a di- hydrate, or a tri-hydrate. All solvated forms and unsolvated forms of the compounds described herein are within the scope of the invention.

Polymerisable Monomers

The present invention relates to a polymerisable monomer of formula (1):

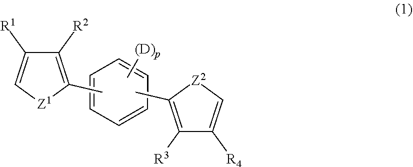

wherein p, R¹, R², R³, R⁴, D, Z¹, Z² and R^{a} are as defined herein.

The polymerisable monomer comprises a central benzene ring substituted with two heteroaryl ring systems. The heteroaryl ring systems are each independently a pyrrole ring system or a thiophene ring system. In some embodiments the pyrrole ring system is a pyrrole ring and the thiophene ring system is a thiophene ring or 3,4-ethylenedioxythiophene (EDOT) ring. The heteroaryl ring systems may be the same or different. In some embodiments the two heteroaryl ring systems are both pyrrole rings or both thiophene rings.

The monomers are capable of polymerising via dehydrogenation of hydrogen atoms at the 5-positions of each the heteroaryl ring systems. The polymerisable monomers may have a polymerisation oxidation potential from about 0 to about 1.0 vs. Ag/AgCl (3 M KCl). For example from about 0.2 to 1.0, 0.3 to 1.0, 0.4 to 1.0, 0.5 to 1.0, 0.6 to 1.0, 0.7 to 1.0, 0.8 to 1.0, 0.2 to 0.9, 0.3 to 0.9, 0.4 to 0.9, 0.5 to 0.9, 0.6 to 0.9, 0.7 to 0.9, or 0.8 to 0.9V vs. Ag/AgCl (3 M KCl). Advantageously, the inventors have found that in some embodiments polymerisable monomers of the formula (1) have a polymerisation potential of from about 0.6 to about 1.0 vs. Ag/AgCl (3 M KCl).

The polymerisation potential of the monomer is preferably sufficiently low that the probe attached to the monomers is not oxidized during the polymerisation reaction.

The heteroaryl ring systems may be unsubstituted or substituted with one or more electron withdrawing or electron donating groups. Electron withdrawing or electron donating groups may be selected such that the monomer has a polymerisation potential within a predetermined range, for example from about 0.6 to about 1.0 V vs. Ag/AgCl (3 M KCl). In various embodiments R¹ and R² together and/or R³ and R⁴ together represent an electron withdrawing group or an electron donating group. For example R¹ and R² and/or R³ and R⁴ may represent a moiety that together with the atoms to which they are attached forms a heterocyclic or carbocyclic ring fused to the pyrrole or thiophene ring system, such as the —OCH₂CH₂O— moiety present in 3,4-ethylenedioxythiophene (EDOT).

In various embodiments, R¹ and R⁴, R² and R³, and Z¹ and Z² in the heteroaryl ring systems are identical such that the heteroaryl ring systems are identical.

In exemplary embodiments, the two heteroaryl ring systems are attached to the benzene ring in a 1,4-relationship.

The benzene ring of the polymerisable monomer may be substituted with one or two D groups, depending on p. In exemplary embodiments, p is 2 and the two D groups are attached to the benzene ring in a 1,4-relationship.

D at each instance of p is a group of the formula -L-P^{x}, wherein L is a bond or a linker group, and P^{x} is a probe capable of binding one or more nucleic acids or comprising a nucleic acid or an analogue thereof. Preferably, the probe is capable of binding one or more nucleic acids in a sequence-specific manner, for example hybridizing with one or more oligonucleotides.

The probe may comprise a nucleic acid or a functional analogue thereof, for example a single or double stranded oligonucleotide, polynucleotide or analogue thereof.

In some embodiments the probe may comprise a single or double stranded oligonucleotide, or single or double stranded polynucleotide.

In various embodiments the probe may comprise a single stranded oligonucleotide or single stranded polynucleotide.

In exemplary embodiments the probe may comprise a single stranded oligonucleotide.

As used herein, a “functional analogue” of a nucleic acid refers to a substrate that differs from the nucleic acid of which it is an analogue, but is capable of binding the same target that the nucleic acid is capable of binding or adapted to bind. A functional analogue may be capable of producing a detectable signal on binding of the target comparable to that provided on binding of the target by the nucleic acidof which it is an analogue, for example at least 50, 55, 60, 65, 70, 75, 80, 85, 90, 95, 97, 98, or 99% of the signal using the nucleic acid of which it is an analogue. As described herein, the detectable signal produced on binding of a target by a probe may be a measurable change in an electrochemical property of a polymer formed from the polymerisable monomers. Examples of functional analogues of nucleic acids include but are not limited to peptide nucleic acids and the like. Functional analogues of nucleic acids can employ any backbone and any sequence capable of resulting in a probe that hybridizes to complementary DNA and/or RNA. Examples of suitable backbones include, but are not limited to, phosphodiesters and deoxyphosphodiesters, phosphorothioates and deoxyphosphorothioates, 2′-O-substituted phosphodiesters and deoxy analogs, 2′-O-substituted phosphorothioates and deoxy analogs, morpholino, 2′-O-alkyl methylphosphonates, 3′-amidates, MMI, alkyl ethers, in addition to peptide nucleic acids.

In some embodiments the probe comprises a single or double stranded nucleic acid, oligonucleotide, or polynucleotide, or an analogue thereof. An oligonucleotide or polynucleotide may comprise from 10 to about 60 nucleotide residues, for example from 10 to about 50, from 10 to about 40, from 10 to about 30, from 10 to about 20, from 15 to about 50, from 15 to about 50, from 15 to about 40, from 15 to about 30, from 13 to about 30, from 15 to about 20, from 20 to about 60, from 20 to about 50, from 20 to about 40, from 20 to about 30, from 30 to about 60, from 30 to about 50, from 30 to about 40, from 40 to about 60, or from 40 to about 50 nucleotide residues. In certain embodiments, an oligonucleotide or polynucleotide may comprises less than 60, less than 50, or less than 40 nucleotide residues. Probes comprising nucleic acids or analogues thereof are commercially available or may be prepared by methods well known in the art.

The probe may be adapted to bind or capable of binding one or more targets other than, or comprising moieties other than, nucleic acids. For example, the probe may comprise an aptamer. Aptamers are single stranded DNA or RNA capable of binding pre-determined targets with both high specificity and affinity, in a manner similar to antibodies. Pre-determined aptamer targets can vary in structure and include, but are not limited to, proteins, peptides, ions and small molecules.

The specificity of the binding of an aptamer may be defined in terms of the dissociation constant Kd of the aptamer for its target. Aptamers can have high affinity with Kd range similar to antibody (pM to nM) and specificity similar/superior to antibody. Aptamers, their uses, and manufacture are described, for example, in U.S. Pat. Nos. 5,840,867, 6,001,648, 6225,058, 6,207,388 and U.S. patent publication 20020001810, the disclosures of all of which are incorporated by reference in their entireties.

A probe may be functionalized to facilitate attachment to the benzene ring either directly or via a linker group. Suitably functionalized probes are readily commercially available or can be prepared by synthetic methods well known in the art. For example, the probe may be amino functionalised, such as the single stranded oligonucleotide probes used in the Examples described below.

The linker group is a group that provides spacing between the benzene ring of the monomers, which on polymerisation form the conductive backbone of the polymer, and the probe. The linker group is typically covalently bound to both the benzene ring and the probe.

The structure of the linker group is not particularly limited. Preferably, the linker allows detection of a signal produced on binding of a target by a probe. Suitable linkers include those capable of transducing a detectable signal, such as an electrochemical signal, between the probe and a polymer formed from the monomers on binding of a target by a probe.

The linker group may be adapted to locate the probe at a predetermined distance from the conjugated backbone of a polymer formed from the monomers to optimize binding of the target by the probe, for example by hybridizing and/or otherwise interacting through non-covalent bonding and the like. It will be appreciated that there is less steric hindrance on formation of a probe-target complex when a linker is longer. However, transduction of the signal produced on binding of a target by the probe may be reduced using a longer linker. Additionally, longer linkers may cause steric hindrance during polymerisation of the monomers.

Linker groups of various lengths can be employed. In some embodiments, the linker group is from about 1 to 15 atoms in length. The atoms of the 1 to 15 atom length of the linker group may be selected from C, N, O, and S, provided that the linker group is stable.

In certain embodiments the linker group has the formula:

—X¹—[(CH₂)ₘ—X²]ₓ—(CH₂)ₙ—X³—

wherein X¹, X², X³, m, x and n are as defined herein.

In various embodiments, the linker is of the formula:

—O—(CH₂)ₙ—X³—

wherein X³ and n are as defined herein.

X³ is a functional group through which the probe is attached. The functional group may be formed, as described below, by the reaction of a probe and a monomer containing a linker group precursor that forms the linker group on reaction with the probe. It will be apparent that the functional group may comprise atoms derived from both the probe and the precursor of the linker group. For example, reaction of an amine functionalized oligonucleotide probe with a linker group precursor comprising a carboxylic acid under suitable peptide coupling conditions provides a linker group wherein X³ is —C(O)NH—. The nitrogen atom of the amide group is derived from the amine group of the amine functionalized probe and the carbonyl group is derived from the carboxylic acid of the linker group precursor.

The nature of the functional group of X³ may thus be determined by the crosslinking reaction used to attach the probe. As described herein, a wide range of crosslinking reactions are suitable.

In exemplary embodiments X³ is —C(O)NH—.

In the polymerisable monomers, the central benzene ring and the two heteroaryl ring systems attached thereto together form a conjugated system comprising alternating single and multiple bonds. In such systems, conjugation is the interaction of one p-orbital with another across an intervening σ-bond. Polymerisation of the monomers can provide intrinsically conducting polymers—that is, organic polymers that conduct electricity. In the polymers, monomer units are bound together to form a conjugated backbone. Due to the conjugation of the backbone, the polymers are electrically conductive. The polymerisable monomers described herein may be prepared by synthetic routes including processes analogous to those well known in the art, such as those described in the Examples below.

The starting materials may be readily available from commercial sources or may be prepared by using methods well known in the art.

Synthetic chemistry transformations and methodologies useful for preparing the compounds described herein include those described in R. Larock, Comprehensive Organic Transformations (1989), which is incorporated herein by reference. The method used depends on the structure of the compound.

Preparation of the compounds may involve the protection and deprotection of various chemical groups. The need for protection and deprotection, and the selection of appropriate protecting groups, can be readily determined by a person skilled in the art. For example, nitrogen protecting groups useful herein include but are not limited to tert-butyloxycarbonyl (Boc), fluorenylmethyloxycarbonyl (Fmoc), carboxybenzyl (CBz), benzyl, and 2-trimethylsilylethyoxymethyl (SEM). Protecting groups for protecting reactive functional groups are well known in the art, as are methods for their introduction and removal (see, for example, P. J. Kocienski, Protecting Groups, Georg Thieme Verlag Stuttgart, New York, 1994; and T. W. Greene and P. G. M. Wuts, Protective Groups in Organic Synthesis, 2d. Ed. (1991), both of which are incorporated herein by reference).

As shown in scheme A below, monomers of the formula (1) can be prepared by reacting a probe (P^{x}) with a monomer of formula (1e), wherein FG is a functional group reactive with the probe and L^{x} is a bond or a linker group precursor that forms the linker on reaction with the probe. In various embodiments, FG is a group that forms X³ on reaction with the probe and L^{x} represents the remainder of the linker group.

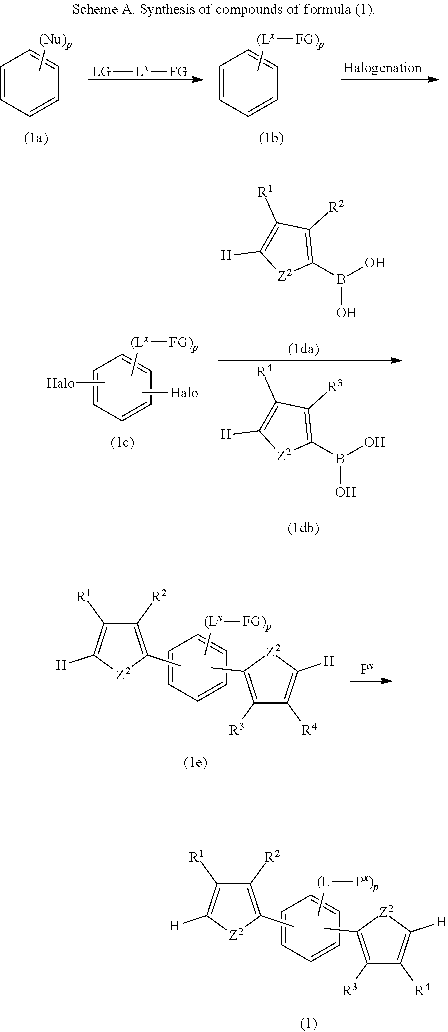

Any suitable crosslinking reaction may be used to attach the probe. Suitable crosslinking reactions include those described in the Thermo Scientific Pierce Crosslinking Technical Handbook 2009, Thermo Fisher Scientific. To facilitate attachment to the monomer, the probe may be functionalized with a suitable reactive functional group. In various embodiments, FG in the monomer of formula (1e) and the reactive functional group of the probe may be selected from the pairs of reaction partners listed in the following Table 1 to provide monomers of the formula (1) with the X³ group indicated. Such crosslinking reactions are well known in the art.

TABLE 1
Examples of pairs of reaction partners that may be used to form the X³
group. R^{v} and R^{w} are at each instance independently H or C₁₋₆ alkyl, preferably H. R^{c}
is C₁₋₆ alkyl, preferably methyl.

| FG | Probe functional group | X³ |
|---|---|---|
| —CO₂H | H₂N— | —C(O)NH— |
| —NH₂ | HO—C(O)— | —NHC(O)— |
| —CO₂H | HO— | —C(O)O— |
| —OH | HO—C(O)— | —OC(O)— |
| —SH | HS— | —SS— |
| —CHO | H₂NNHC(O)— | —CH═NNHC(O)— |
| —C(O)NHNH₂ | HC(O)— | —C(O)NHN═CH— |
| —NHC(O)CR^{v}R^{w}I | HS— | —NHC(O)CR^{v}R^{w}S— |
| —SH | ICR^{w}R^{v}C(O)NH— | —SCR^{w}R^{v}C(O)NH— |
| —C(═NH)OR^{c} | H₂N— | —C(═NH)NH— |
| —NH₂ | R^{c}OC(═NH)— | —NHC(═NH)— |
| —CHO | H₂N— | —CH═N— |
| —NH₂ | HC(O)— | —N═CH— |
| —NH₂ | 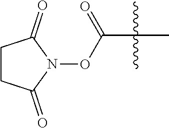 [SMILES: *C(=O)ON1C(=O)CCC1=O] | —NHC(O)— |
| 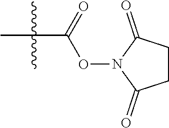 [SMILES: *C(=O)ON1C(=O)CCC1=O] | H₂N— | —C(O)NH— |
| —SH | 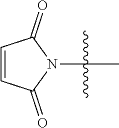 [SMILES: *N1C(=O)C=CC1=O] | 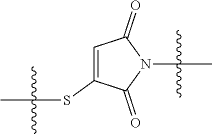 [SMILES: *SC1=CC(=O)N(*)C1=O] |
| 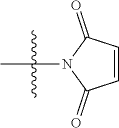 [SMILES: *N1C(=O)C=CC1=O] | HS— | 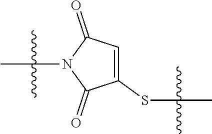 [SMILES: *SC1=CC(=O)N(*)C1=O] |

In other embodiments, X³ may be a group formed by the non-specific reaction of a monomer of the formula (1e) or a probe comprising an azide, for example a phenyl azide, hydroxyphenyl azide, nitrophenyl azide, tetrafluorophenyl azide, with the other reaction partner. It will be appreciated by those skilled in the art that cross-linking reactions involving the use of an azide can result in a number of different products each having different X³ groups.

Other cross-linking reactions and the X³ groups produced thereby will be apparent to those skilled in the art.

Compounds of formula (1e) may be prepared by coupling the two heteroaryl ring systems simultaneously or sequentially to the central benzene ring. The heteroaryl ring systems may be coupled via a transition metal catalysed cross-coupling reaction, for example a Suzuki reaction. The Suzuki reaction typically involves the cross-coupling of a boronic acid or a boronate ester and a halide in the presence of a palladium(0) complex. A Pd(0) complex, such as tetrakis(triphenylphosphine)palladium(0), may be provided directly to the reaction or may be formed in situ (e.g. from Pd(OAc)₂). Any suitable palladium-based catalyst known in the art may be used. As shown in scheme A, compounds of formula (1e) may be prepared by coupling a suitable dihalide (1c), such as a dibromide or diiodide, with boronic acids comprising the heteroaryl ring systems (1da and 1db). In other embodiments, compounds of formula (1e) may be prepared by coupling a suitable dihalide (1c) with boronates corresponding to the compounds of formula (1da) and (1db) wherein the boronic acid moiety is replaced with a boronate ester (such as a pinacol ester). In certain exemplary embodiments (1da) and (1db) are identical. The Suzuki reaction may be carried out in a liquid solvent in which the boronic acid(s) are soluble, for example ethanol or n-butanol. The reaction is typically carried out at elevated temperature, for example 70° C. or 110° C., until complete. Additives that enhance the rate of reactivity of the palladium complex, for example SPhos, may be included in the reaction mixture.

The reaction conditions for the Suzuki reaction may be varied depending on the nature of the other groups present in the molecule, in particular the -L^{x}-FG groups. FG may need to be protected with a suitable protecting group during the Suzuki reaction and then deprotected prior coupling the probe.

To increase the rate of the Suzuki reaction and/or to avoid or limit the formation of mono-addition products, the reaction conditions may be varied for example by increasing the reaction time and/or by adding more (for example, a stoichiometric excess) of one or more or of the reactants to the reaction mixture.

Various compounds of the formula (1da) and (1db) are commercially available. Others can be readily prepared by literature methods or adaptations thereof.

Compounds of the formula (1c) can be prepared by halogenation of the corresponding compound of formula (1b). For example, as described in the Examples, a benzene ring 1,4-disubstituted with two protected -L^{x}-FG groups may be treated with iodine, in the presence of Hg(OAc)₂, to provide the corresponding 1,4-diiodide (i.e. a diiodide wherein the two iodine atoms are also in a 1,4-relationship). In other examples the diiodide may be formed using a mixture of periodic acid and iodine or using iodine monochloride. Other iodination conditions will be apparent to a person skilled in the art. The halogenation conditions used may be varied depending on the nature and/or substitution pattern of the L^{x}-FG groups present.

Compounds of the formula (1b) may be commercially available or readily prepared by known methods. For example, as shown in scheme A, compounds of formula (1b) may be prepared by reacting a compound of the formula LG-L^{m}-FG with a compound of the formula (1a). Nu is nucleophile and LG is a suitable leaving group. L^{m} is a group that forms L^{x} on displacement of the leaving group by the nucleophile. Examples of suitable leaving groups include sulfonates, for example tosyl and triflate groups, and halides, for example iodides and bromides. In some embodiments, the nucleophile is an alcohol, amine, or thiol.

Other methods of preparing compounds of formula (1b) will be apparent to those skilled in the art. The method used will depend on the structure of the -L^{x}-FG group(s). For example, compounds of formula (1b) may be prepared by reacting a compound of the formula Nu-L^{m}-FG and a compound of formula (1a) wherein each Nu is replaced with LG. Alternatively, compounds of formula (1b) may be prepared by reacting a compound of formula Nu-FG and a compound of formula (1a) wherein each Nu is replaced with a -L^{m}-LG group.

Compounds of formula (1b) may also be prepared by reacting a compound of the formula LG-L^{p}-FG and a compound of the formula (1a) wherein each Nu is replaced with a -L^{o}-Nu, and wherein L^{p} and L^{o} are each groups representing a portion of L^{x} which on displacement of the leaving group by the nucleophile combine together to form L^{x}. Alternatively, compounds of formula (1b) may be prepared by reacting a compound of the formula Nu-L^{p}-FG and a compound of the formula (1a) wherein each Nu is replaced with a -L^{o}-LG.

The reactive functional group of each -L^{x}-FG group may be protected with a protecting group during preparation of the polymerisable monomers. The protecting group used depends on the reactive functional group. As noted above, protecting groups and methods for their introduction and removal are well known in the art. For example, carboxylic acid functional groups may be protected as esters.

Similarly, where one or both of the heteroaryl rings attached to the central benzene ring is a pyrrole ring system, the nitrogen atom of the pyrrole ring may be protected during synthesis of the polymerisable monomers, for example using a Boc group during the cross-coupling reaction.

The reactive functional group of an -L^{x}-FG group in the polymerisable monomers may be reacted to convert the reactive functional group into another reactive functional group for crosslinking with the probe. Methods for the converting reactive functional groups into other reactive functional groups are well known in the art.

The products of each reaction in the synthetic sequence may be isolated and/or purified from the reaction mixture by standard methods known in the art or, where possible, used without purification.

Polymerisable monomers of the formula (7) as described herein may be prepared by analogous synthetic routes. In such compounds, the central benzene ring is substituted with one or two Y groups, rather than the D groups present in the compounds of formula (1).

Conducting Polymers

The present invention also relates to a conducting polymer comprising a monomer unit of the formula (2)

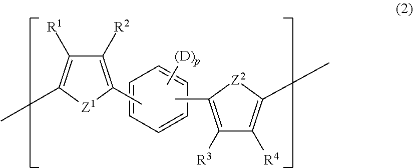

wherein p, R¹, R², R³, R⁴, D, Z¹, and Z² are as defined herein.

The polymer may be homopolymer comprising just one type of monomer unit or may be a copolymer comprising monomer units of formula (2) and one or more monomer units different to the monomer unit of formula (2).

As used herein, the term “copolymer” refers to a polymer comprising two or more, for example three or more, different monomer units—that is, two or more monomer units having different structures. Copolymers of two different monomers may be referred to as bipolymers and copolymers of three different monomers may be referred to as terpolymers. The different monomer units may be randomly distributed throughout the polymer or in an ordered arrangement, for example in the form of blocks, depending on how the copolymer is formed.

The one or more monomer units different to the monomer unit of the formula (2) in the copolymers may be a monomer unit of the formula (3), (4), (5) or a mixture of any one or more thereof as described herein. It will be appreciated that these monomer units are derived from monomers of thiophene, pyrrole, and 3,4-ethylenedioxythiophene (EDOT), respectively. Such monomer units may be present in the polymer in any suitable ratio with the monomer unit(s) of formula (2).

In certain embodiments the one or more monomer unit different to the unit of formula (2) is a monomer unit of the formula (6)

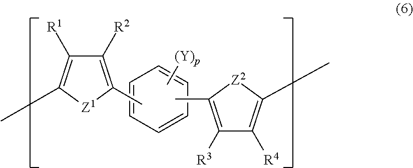

wherein Y, p, R¹, R², R³, R⁴, Z¹, and Z²are as defined herein.

In certain embodiments, the monomer unit of formula (2) and monomer unit of formula (6) are identical, except for the D and Y groups, which are different.

The monomer units of the polymers described herein are linked together to form conjugated backbone. The backbone can comprises pyrrole ring systems, thiophene ring systems, or a mixture of pyrrole and thiophene ring systems. In certain exemplary embodiments, the pyrrole or thiophene ring systems in the backbone are either all pyrrole ring systems or all thiophene ring systems. Such backbones may be easier to form because the polymerisation oxidation potentials of the corresponding monomers are similar. The pyrrole or thiophene ring systems may be the same or different. For example, a polymer backbone wherein the pyrrole or thiophene ring systems are all thiophene ring systems may comprise a mixture of thiophene rings and 3,4-ethylenedioxythiophene rings.

In certain embodiments, each Y is independently a water solubilising and/or protein repellent group. Examples of such groups include polyethers and polyether alcohols, such as polyethylene and polypropylene glycols and glycol ethers. In exemplary embodiments Y is a polyether. Without wishing to be bound by theory, the inventors believe protein repellant groups such as polyethylene glycols and glycol ethers in copolymers of the invention are capable of blocking the adsorption of proteins on the surface of the polymer by forming a layer of well-solvated brushes on the surface of the polymer that creates a high activation barrier for proteins to adsorb.

The one or more monomer units different to the monomer unit of the formula (2) in the copolymers can provide spacing between monomer units of formula (2), which may reduce steric effects detrimental to formation of probe-target complexes. The ratios of various monomers in the copolymer may be varied to optimize the properties of the polymer for a desired application, for example the ability of probes in the polymer to bind a target or the signal produced on binding of a target by probe, or to optimize solubility in solvents used in sensor preparation. In certain embodiments, the conducting polymer comprises monomer units of formula (2) and monomer units of the formula (6) in a ratio of from about 10.1 to 1:1,000, 10:1 to 1:500, 10:1 to 1:100, 1:1 to 1:100, 1:1 to 1:50, 1:1 to 1:5, or 1:2 to 1:4, or about 1:3.

As described herein, the monomer units of the polymers form a conjugated backbone that enables the polymer to conduct electricity. The ability of the conducting polymers to conduct electricity enables electrochemical detection of the recognition of a target by probes attached to the polymer. The conducting polymers described herein may have a conductivity in S/cm of at least about 1×10⁻⁹.

The present invention also relates to a method of making a conducting polymer of the present invention, the method comprising:

(a) providing a polymerisable monomer of the formula (1): and

(b) polymerising the monomer to provide a conducting polymer comprising a monomer unit of the formula (2).

The present invention also relates to conducting polymers made by the method.

Copolymers may be made by copolymerising the monomer of formula (1) and at least one additional polymerisable monomer, for example thiophene, pyrrole, 3,4-ethylenedioxythiophene (EDOT), or a monomer of the formula (7):

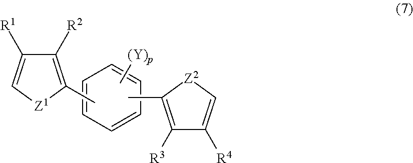

wherein Y, p, R¹, R², R³, R⁴, Z¹, and Z² are as defined herein.

Polymerisation may carried out by any suitable method, as described herein. Without wishing to be bound by theory the inventors believe that in certain embodiments having D groups located on the central benzene ring of the polymerisable monomers described herein allows polymerisation at the alpha positions of the thiophene/pyrrole ring system with relative ease.

Sensors and Sensor Systems

The present invention also relates to a sensor comprising a substrate having a surface coated with a conducting polymer of the invention. The substrate may provide a solid surface that supports the attached coating of the conducting polymer. The substrate may for example be flexible or rigid.

In various embodiments, the substrate comprises at least one electrode having a surface coated with the conducting polymer. In certain embodiments, the substrate is or consist of an electrode.

Signals produced on binding of a target by a probe can be transduced through the conducting polymer to the electrode. Thus, changes in the electrochemical properties of the conducting polymer may be measured via the electrode on which it is coated.

In various embodiments, the substrate comprises a plurality of electrodes, each electrode having a surface coated with a conducting polymer of the invention At least two of the electrodes are coated with conducting polymers having different probes. In various embodiments, each electrode is coated with a conducting polymer having a different probe. The electrodes are separated from each other by an insulating material. The electrodes may be in the form of an array, for example a microelectrode array. An array may comprise a plurality, for example 2-100, 5-100, 5-90, 5-80, 5-70, 5-60 or 5-50 of electrodes coated with different probes in an ordered arrangement, for example a two dimensional arrangement of columns and rows. Each electrode is independent from the others such that binding a target to a probe of one electrode can be detected independently of any interactions with probes at other electrodes. Processes for the fabrication microelectrode arrays are well known the art including for example photolithography, etching and screen printing.

In some embodiments the sample may be in a sample holder or sampling device fabricated onto one or more electrode.

In some embodiments the sample may not be in a receptacle or sample holder, but may be retained on the surface of one or more electrodes, for example by the formation of a capillary gap above one or more electrodes.

The electrode(s) coated with the conducting polymer(s) may be a gold (screen printed gold), platinum, carbon (e.g. glassy or screen printed carbon), stainless steel, indium tin oxide (ITO), or doped silicon wafer electrode. Other suitable electrodes will be apparent to those skilled in the art. Conveniently, arrays of electrodes, such as screen printed carbon electrodes, are readily commercially available.

Screen printed electrode(s), such as screen printed carbon electrode(s), useful herein may be surface modified prior to formation of the coating of the conducting polymer(s) by a treatment that increases the sensitivity of the electrode to detection of the target. Suitable surface treatments include but are not limited to laser glazing and plasma treatment. In some embodiments, laser glazing using a high-power laser such as an eximer laser converts a screen-printed carbon surface into one resembling glassy carbon.

The coating of the conducting polymer is typically in the form of a thin film. In certain embodiments, the thin film is porous. The thickness of the coating or film can range from 1 to about 1000 nm, for example from about 100 to about 1000 nm, for example 20 to about 1000 nm, for example from about 500 to about 1000 nm, for example from about 7500 to about 1000 nm, for example from about 900 to about 1000 nm, for example from about 100 to about 900 nm, 250 to about 900 nm, for example from about 500 to about 900 nm, for example from about 750 to about 900 nm, for example from about 100 to about 800, 250 to about 800 nm, for example from about 500 to about 800 nm, for example from about 7500 to about 800 nm, for example from about 100 to 700 nm, for example from about 250 to about 700 nm, for example from about 500 to about 700 nm, for example from about 100 to about 600 nm, for example from about 250 to about 600 nm, for example from about 500 to about 600 nm, for example from about 100 to about 500 nm, for example from about 250 to about 500 nm.

The present invention also provides a sensor system comprising a sensor of the present invention and a detector for determining the presence or absence or amount of a target, for example a detector capable of detecting binding of the target by the probe. Binding of the target by the probe may be sequence specific. For example, in some embodiments sequence specific binding may be by nucleic acid hybridization. It will be appreciated by a person skilled in the art that binding may be achieved by hybridization of a target with less than 100% complementarity to the sequence of the probe by hybridizing under stringent conditions. For example, hybridization under stringent conditions may involve hybridization at a specific temperature and salt concentration. The stringent conditions required for hybridization may be determined by hybridizing under less stringent conditions initially and adjusting the conditions as desired until the stringent hybridization conditions are identified.

The detector is selected based on the desired detection method. The detector may be capable of measuring an electrochemical property of the conducting polymer. For example, the detector may detect by ampereometry, cyclic voltammetry, conductometry, electrochemical impedance spectroscopy, or any other suitable method known in the art. In certain exemplary embodiments, the detector is capable of measuring the impedance of the conducting polymer, for example by electrochemical impedance spectroscopy.

To measure an electrochemical property of the conducting polymer, the detector may be connected in an electrical circuit with an electrode coated with the conducting polymer—the working electrode—and a counter electrode, and optionally a reference electrode. Thus a sensor, for example the substrate of a sensor, or a sensor system may comprise a counter electrode and optionally a reference electrode. The counter electrode and reference electrode, if applicable, may be selected based on their compatibility with the working electrode and with the sample and measurement procedure adopted. Suitable counter electrodes and reference electrodes will be apparent to those skilled in the art.

The system may further comprise a positive control. For example in some embodiments the system may further comprise a positive control sample comprising a target, which probes of the conducting polymer(s) are capable of binding.

The system may further comprise a redox couple, such as ferro-ferricyanide.

Without wishing to be bound by theory the inventors believe that the electrochemistry of such redox couples on conducting polymers is sensitively affected by binding of or hybridization of a target with a probe.

The present invention also relates to a method of making a sensor comprising:

(i) providing a monomer of the formula (1) as defined herein;

(ii) providing a substrate; and

(iii) polymerising the monomer of the formula (1) to provide a conducting polymer and depositing the conducting polymer on a surface of the substrate to provide a coating of the conducting polymer on the surface of the substrate; or (iii) depositing the monomer of the formula (1) on a surface of the substrate and polymerising the monomer to provide a coating of a conducting polymer on the surface of the substrate.

The present invention also relates to a sensor made by the method.

A plurality of monomers of formula (1) (i.e. a plurality of different monomers) may be be polymerised to make a sensor to provide a sensor comprising a plurality of conducting polymers (i.e. a plurality of different conducting polymers) at separate, predetermined locations on a surface of the substrate, such as on a surface of an electrode. In some embodiments a plurality of monomers of formula (1) and one or more additional monomers, for example a monomer of formula (7), may be polymerised to make a sensor. Each of monomer of the plurality of monomers differ in structure, each monomer preferably comprising a different probe.

Each monomer may be polymerised and the resultant conducting polymer deposited at a separate, predetermined location on a surface of the substrate to provide a coating of the conducting polymer at the location. Alternatively, each monomer may be deposited at a separate, predetermined location on a surface of the substrate and polymerised to provide a coating of the conducting polymer at the location. The monomer or conducting polymer deposited may be in the form of a solution or suspension comprising the monomer or conducting polymer and optionally one or more suitable solvents. Examples of suitable solvents include volatiles organic solvents, such as DMF or THF, and may include a buffer such as PBS. Following deposition, the one or more optional solvents may be removed, for example by evaporation. Where a monomer is deposited, the monomer may be polymerised prior to, during, or after removal of the one or more optional solvents.

At least two of the locations are coated with a conducting polymer having a different probe. Preferably, each location is coated with conducting polymer having different probe.

The substrate may comprise a plurality of electrodes. Each location may comprise an electrode and the coating of the conducting polymer at each location may be on a surface of the electrode at that location.

Sensors comprising a plurality of conducting polymers with different probes at separate, predetermined locations are capable of simultaneously detecting a plurality of different targets. The separate, predeteremined locations at which the conducting polymers are coated may be selected such that the sensor comprises an array of conducting polymers comprising different probes in an ordered arrangement, for example a two dimensional arrangement of columns and rows. The array may, for example be a DNA microarray.

The monomers or polymers may be deposited on the surface by any suitable method. Where a plurality of monomers or polymers are deposited at separate, predetermined locations on the substrate, such as on separate electrodes, the monomers or polymers may be deposited simultaneously or sequentially. For example, an array of pipettes (for example, an array of micropipettes), each pipette comprising a solution of a monomer or polymer bearing a different probe, may be used to simultaneously deposit the monomers or polymers on the surfaces of a corresponding array of electrodes.

Polymerisation of the monomers may be carried out by any suitable method, for example chemical oxidative polymerisation or electropolymerisation.

In chemical polymerisation, the monomers are treated with an extraneous chemical oxidizing agent, such as ammonium persulfate or iron(III) chloride, to oxidise and polymerise the monomers. The reaction may be carried out at ambient temperature in a suitable solvent, for example nitromethane. Chemical polymerisation is commonly used for the preparation of conducting polymers in solution or bulk solids.

In various embodiments, the monomers are polymerised by electroless oxidative polymerisation, wherein the oxidant is oxygen or hydrogen peroxide. Compared to conventional chemical polymerisation employing other oxidants, oxidative polymerisation using oxygen or hydrogen peroxide as the oxidant is cleaner as the by-product produced is water. In certain embodiments, the oxidant may be oxygen in air or oxygen dissolved in a solution of the monomer(s).

An oxygen reduction catalyst where the oxidant is oxygen or hydrogen peroxide reduction catalyst where the oxidant is hydrogen peroxide can be used to increase the rate of polymerisation. Any suitable oxygen and hydrogen peroxide reduction catalysts may be used. A large number of such catalysts are known in the art. Suitable catalysts include, but are not limited to, elemental Pt, Pd, Ru, or Ir; oxides of Pt, Pd, Ru, or Ir; and carbon, for example carbon nanotubes, fullerines, or graphene; and mixtures any two or more of such catalysts. In certain embodiments, the catalyst is platinum or palladium. The catalyst may be in any suitable form. In certain embodiments, the catalyst is in the form of nanoparticles.

The polymerisable monomers described herein may have an oxidation polymerisation potential sufficiently low that polymerisation spontaneously proceeds in the presence of oxygen (e.g. air or dissolved oxygen) or hydrogen peroxide. However, in certain embodiments, the polymerisable monomers are stable to polymerisation by oxygen or hydrogen peroxide in the absence of an oxygen or hydrogen peroxide catalyst for a period of time of at least 4 hours, for example for at least 4, 6, 8, 10, 12, 18, 24, 30, 36, 42 or 48 hours and useful ranges may be selected from any of these values. In such embodiments, the rate of polymerisation in the absence of the catalyst is so slow that no appreciable amounts of polymer are formed over the period of time, for example less than about 5, 4, 3, 2, or 1 mol % of the monomer(s) polymerise. Monomers having such stability thus and can be stored in solution for later polymerisation. In various embodiments, Z¹ and Z² in the monomers stable to polymerisation in absence of the catalyst are each S.

A solution comprising the polymerisable monomer(s) and optionally one or more suitable solvents, the oxidant, and optionally the catalyst may be admixed in any order to polymerise the monomers and form a solution or suspension of a conducting polymer, which may then be deposited on the substrate. In certain embodiments where the oxidant is oxgen, it may not be necessary to admix the oxidant - the oxidation may be carried out by oxygen dissolved in the solution comprising the monomer(s) or by oxygen in the air. In certain embodiments, where the oxidant is hydrogen peroxide an aqueous solution of hydrogen peroxide is admixed with the polymerisable monomer(s) and optionally the catalyst.

Alternatively, a solution comprising polymerisable monomer(s) and one or more suitable solvents can be deposited on a surface of a substrate or electrode that consists of or comprises the catalyst. In certain embodiments where the oxidant is dissolved oxygen or hydrogen peroxide, the solution comprising the polymerisable monomers further comprises the oxidant. The catalyst catalyses polymerisation of the monomers such that a coating of a conducting polymer is formed on the surface. In various embodiments, the monomer solution is deposited on a surface of an electrode consisting of the catalyst, for example a Pt electrode. In other embodiments, the monomer solution is deposited on a surface of comprising nanoparticles of the catalyst, for example Pt nanoparticles. The nanoparticles may be deposited on the surface by any suitable method. In various embodiments, nanoparticles of the catalyst, for example Pt nanoparticles, are deposited on a surface of an electrode by electrodeposition or by deposition and evaporation of a colloidal dispersion of the nanoparticles onto the electrode.

The inventors have found that in certain embodiments wherein oxygen is the oxidant, thin films of conducting polymer rapidly form when monomers are deposited on a surface comprising an oxygen reduction catalyst. Advantageously, in certain embodiments the rate of polymerisation may be such that carrying out the electroless oxidative polymerisation reaction for a period of time from about 1 to 120 seconds may be sufficient to provide a polymer film having a thickness from about 5 nm to 10 μm, preferably from 5 to 100 nm.

In the case of electropolymerisation, polymer film thickness may be controlled, for example by controlling the applied potential and the length of time for which the polymerisation potential is applied and controlling the concentration of the monomer solution.

In the case of electroless polymerisation, polymer film thickness may be controlled, for example by controlling the concentration of the monomer solution and the length of time for which polymerisation is carried on. Electroless polymerisation may be stopped, for example, by washing away a monomer solution with a solvent that does not dissolve the polymer.

Electropolymerisation can be used for deposition of films of conducting polymers on conducting substrates. In electropolymerisation, polymerisation occurs on the surface of an electrode (the working electrode). The conducting polymer forms as a film on the electrode surface. The electrode may be a gold (e.g. screen printed gold), platinum, carbon (e.g. glassy or screen printed carbon), stainless steel, indium tin oxide (ITO), or doped silicon wafer electrode. The electrode is contacted with a solution comprising one or more monomers and one or more suitable solvents. For example, the solution may be deposited on a surface of the electrode, or the electrode may be immersed in the solution. The solution may further comprise a buffer, such as PBS.

To polymerise the monomers and form a coating or thin film of the conducting polymer a potential is applied between the working electrode and a counter electrode. A reference electrode may also be empolyed. Suitable counter and reference electrodes will be apparent to those skilled in the art.

Any suitable potential may be applied to polymerise the monomers. Depending on the polymerisation potential of the monomers, the electropolymerisation may be carried out at potential from about 0 to about 1.0V vs (Ag/AgCl (3M KCl), for example from about 0.6 to about 1.0. The potential applied is preferably such that the probe attached to the monomers is not oxidized during the polymerisation reaction.

Various parameters of the electropolymerisation can be used to control the thickness of the conducting polymer coating and the speed of polymerisation. Advantageously, the inventors have found that in some embodiments conducting polymer films can be formed rapidly, in less than 1 second by electropolymerisation at a potential of 0.8V. Such a rapid speed is useful for the production of sensors and sensor arrays on a commercial scale. In various embodiments, the electropolymerisation provides a polymer film having a thickness of from about 5 nm to 10 μm, preferably between 5 nm and 100 nm, when carried out for a period of time from about 0.1 seconds to about 10 seconds.

Conveniently, a monomer may be deposited on an electrode, and polymerised using, for example a pipette comprising a solution of the monomer and an electrode at the tip of the pipette. The amount of the solution of the monomer is deposited on the surface of the electrode and/or the distance between the tip of the pipette and the surface of the electrode is such that the solution deposited contacts both the tip of the pipetted and the surface of the electrode. The monomer is polymerised on the surface of the electrode by applying potential between the electrode of the substrate and the electrode at the tip of the pipette. An array of such pipettes (for example, an array of micropipettes), each pipette comprising a solution of a monomer bearing a different probe, may be used to simultaneously deposit and subsequently polymerise the monomers on the surfaces of a corresponding array of electrodes.

Detecting or Amplifying Targets

In another aspect, the present invention provides a method for determining the presence or absence or amount of a target in a sample, the method comprising:

(a) contacting
(1) a sample which may comprise a target, and
(2) a sensor or sensor system according to the invention; and

(b) determining the presence or absence or amount of the target when present in the sample.

In certain embodiments, the determining step comprises detecting binding of the target when present in the sample.

The sensors of the invention may comprise a polymer deposited on the surface of an electrode. One or more monomers of the polymer bear a probe capable of binding or adapted to bind a target. In some embodiments the sensor may be a sensor array comprising a plurality of electrodes, each electrode bearing a different probe, and each capable of binding a different target. Such sensor arrays may be useful as diagnostic tools and in various areas of research, including for example forensics and genome analysis.

The term “sample” as used herein refers to a composition obtained from any source which may comprise a target. A sample may be an environmental, clinical, biological, food, forensic, or other suitable sample. Environmental samples include, but are not limited to, soil, sediment, water, and aerosol samples. A biological sample may be obtained from plants, humans or non-human animals (including vertebrates and invertebrates),In some embodiments, a biological sample may be obtained from microbes (such as cells and viruses). Examples of human samples include, but are not limited to, saliva, sputum, feces, tissue, blood, synovial fluid, spinal fluid, serum, and urine samples. A sample may be purified or unpurified and may be treated or processed prior to contacting with the sensor. For example, a sample comprising cells may be treated to lyse the cells and release DNA and other nucleic acids contained therein. A sample may comprise a plurality of targets (i.e. a plurality of different targets).

In some embodiments, the sample comprises double stranded nucleic acid. In various embodiments, the sample comprises genomic nucleic acid. In some embodiments, the sample comprises a lysate. In various embodiments, the sample comprises a lysate comprising genomic nucleic acid. The lysate may be cell lysate, such as a bacterial cell lysate. A lysate, such as bacterial cell lysate, in addition to nucleic acid, typically comprises protein, lipids, and various other components produced on lysis. Lysis may produce solid components or particles in the lysate, which may be removed, for example by filtration, prior to contacting with the sensor.

Advantageously, the inventors have found that samples comprising lysates, such as bacterial cell lysates, comprising genomic nucleic acid can be used directly in the methods, systems, and apparatus of the present invention without having to first subject the sample to nucleic acid extraction and/or purification to separate the nucleic acid from other cellular components, such as lipid and/or protein. Nucleic acid extraction and/or purification methods are well known in the art and typically involve multiple processing steps, are time consuming, and require various equipment (for example, centrifuge, refrigerator, etc.). Nucleic acid extraction and/or purification methods may comprise treatment with a proteinase, extraction with one or more organic solvents, precipitation of the nucleic acid, and/or purification and/or isolation of the precipitated nucleic acid. In various embodiments, the sample has not been subjected to such nucleic acid extraction and/or purification methods.

The ability to use a lysate as a sample in the present invention, either directly or for example after removal of at least a portion of the solid components or particles produced by lysis, significantly reduces the amount of sample preparation required. As no nucleic acid extraction and/or purification is necessary, the methods, systems, and apparatus can be employed in the field, away from traditional cleanrooms and/or other laboratory facilities necessary for nucleic acid extraction and/or purification, enabling rapid nucleic acid detection on-site.

A sample comprising microbes (for example, cells, such as bacteria, or viruses) comprising a target nucleic acid may be treated to lyse the microbes and release double stranded nucleic acid, for example genomic nucleic acid, contained therein. A sample comprising double stranded nucleic acid may be heated for a period at a temperature sufficient to dissociate the nucleic acid strands. Heating may be carried out prior to brining the sample into contact with the sensor or while the sample is in contact with the sensor. The dissociated nucleic acid strands are contacted with a sensor of the invention, such as a sensor comprising a probe comprising a single stranded oligonucleotide or polynucleotide. Cooling allows annealing of the dissociated single stranded target nucleic acid to the probe of the sensor. Lysing of microbes may be carried out by heating or other suitable means. The heating may be sufficient to lyse the microbes and dissociate the nucleic acid strands of the double stranded nucleic acid released.

Various components to facilitate and/or assist in the detection or quantification of a target may be admixed with a sample, for example buffers, redox couples (such as ferri- and ferro-cyanide), and nucleotides, nucleic acids and enzymes for a nucleic acid amplification reaction (for example, polymerase amplification).

The method comprises contacting the sensor or sensor array to a sample which may comprise one or more targets. Without wishing to be bound by theory, the inventors believe that in some embodiments the one or more targets in the sample bind to probe(s) on the surface of the polymer of the sensor, for example by hybridization and/or otherwise interacting through non-covalent bonding and the like. This interaction produces a detectable signal, such as a change in a property of the conducting polymer resulting from a change in the conformation of a probe and/or an electronic property of the probe. Any suitable method for determining the presence or absence or amount of a target, or binding of a target to a probe may be used. Various methods are known in the art.

Without wishing to be bound by theory, the inventors believe that the charge transfer resistance (R_{CT} ) of the conducting polymer is sensitive to changes in the properties of probes, including changes in the conformation of probes that may result from target binding. A signal produced on a target binding to a probe may be transduced through the probe into the conducting polymer, such that the presence or absence of a target in a sample can be detected by reference to the presence or absence of a detectable change in a property of the polymer.

Signal transduction may occur in a number of ways. For example, in various embodiments binding of a target by a probe may be detected electrochemically. Binding of a target may result in a change in the electronic structure and/or charge distribution near the surface of the conducting polymer that can be detected electrochemically.

In certain exemplary embodiments changes in the electrochemical properties of the conducting polymer may be measured via the electrode on which the conducting polymer comprising the probe is coated, and the detector detects changes in the impedance of the conducting polymer, for example by electrochemical impedance spectroscopy (EIS).

EIS is sensitive to the interaction of targets with the polymer surface and may be used to monitor changes in interfacial charge transfer resistance (R_{CT} ). In one embodiment R_{CT} measurements may be used to determine the amount of one or more targets in a sample, for example by interpolating concentration for a measured R_{CT} from the calibration curve of normalized R_{CT} vs. log c. The concentration for a measured R_{CT} is commonly normalized to the R_{CT} value for the film before incubating with the target, denoted as R_{CT0} .

The sensor and sensor array of the invention includes probes selected based on their target-specificity. For example, a probe may be capable of binding or adapted to bind a nucleic acid target or a targets other than, or comprising moieties other than nucleic acids, such as when the probe is an aptamer. The probe may be capable of binding or adapted to bind protein, peptide, polypeptide or nucleic acid targets, for example DNA, mRNA, tRNA or rRNA.

In exemplary embodiments the probe is a nucleic acid, capable of binding or adapted to bind a nucleic acid target.

In various embodiments nucleic acid targets may include for example specific nucleic acid sequences characteristic of bacteria responsible for sexually transmitted diseases, upper respiratory tract infections and food poisoning, viruses such as HIV and nucleic acid sequences that may be indicative of particular types of cancer such as bladder cancer and breast cancer.

In some embodiments the probe is an aptamer capable of binding or adapted to bind targets other than, or comprising moieties other than nucleic acids. Aptamers are commercially available or can be produced by a process known as SELEX.

In certain embodiments the target may be selected from chemicals that mimic hormones, hormones, naturally occurring phytoestrogens, narcotics and metabolites thereof. Preferably, the non-nucleic acid target is an endocrine disrupting compound, a steroidal sex hormone, or a metabolite or synthetic variant thereof. Preferably, the non-nucleic acid target is selected from 7p-oestradiol (E2); oestrone; oestriol; androstenedione; testosterone; dihydrotestosterone; pregnenolone; progesterone; 17a-hydroxyprogesterone, 7a-ethynylestradiol; isoflavones; lignans; coumestans; organohalides including organochlorines, polychlorinated organic compounds, polychlorobiphenyl (PCB); alkylphenols; alkylphenol ethoxylates; phthalates;

bisphenol-A (BPA); Bis (4-hydroxyphenyl) methane; cholesterol; adenosine; triclosan; or synthetic steroids such as diethylstilboestrol (DES); cocaine, heroin and any metabolites thereof. In certain embodiments, the non-nucleic acid target is selected from 17 -oestradiol, testosterone, progesterone, and adenosine.

In some embodiments, the target may be a hormone or marker of a condition of disease in a body. For example, the probe could be selective for the detection of hormones and/or metabolites to establish fertility, or status in an animal.

In some embodiments, the probe can be selected for the detection of known markers of disease, for example over-expression of a cancer gene to detect cancer, detection of molecules or substrates associated with infection, or to establish levels of specific metabolites associated with a particular condition.

In some embodiments the target may be an ion, such as an ion selected from bromide, cadmium, calcium, cerium, chloride, copper, fluoride, iodide, iron, lanthanum, lead, nitrate, potassium, sodium, strontium, sulphate, and zinc.

In certain embodiments the sensor may comprise a redox couple, such as ferro-ferriocyanide. Without wishing to be bound by theory the inventors believe that the inclusion of a redox couple in the system may lead to increased sensitivity of target detection. In some embodiments the sensor of the invention may be capable of detecting the presence of a target in a sample, wherein the concentration of the target in the sample is less than 10 nM or less than 10 fM, for example less than 1×10⁻⁹, 1×10⁻¹⁰, 1×10⁻¹¹, 1×10⁻¹², 1×10⁻⁹, 1×10⁻¹³, 1×10⁻¹⁴, 1×10⁻¹⁵, 1×10⁻¹⁶, or 1×10⁻¹⁷ M.

The concentration of the target in the sample can vary. In some embodiments, the concentration of the target in a sample is from about 1×10⁻¹ to about 1×10⁻¹⁸, 1×10⁻² to about 1×10⁻¹⁸, 1×10⁻⁴ to about 1×10⁻¹⁸, 1×10⁻⁶ to about 1×10⁻¹⁸, 1×10⁻¹ to about 1×10⁻¹⁷, 1×10⁻² to about 1×10⁻¹⁷, 1×10⁻⁴ to about 1×10⁻¹⁷, 1×10⁻⁶ to about 1×10⁻¹⁷, 1×10⁻¹ to about 1×10⁻¹⁵, 1×10⁻² to about 1×10⁻¹⁵, 1×10⁻⁴ to about 1×10⁻¹⁵, 1×10⁻⁶ to about 1×10⁻¹⁵, 1×10⁻¹ to about 1×10⁻¹³, 1×10⁻² to about 1×10⁻¹³, 1×10⁻⁴ to about 1×10⁻¹³, 1×10⁻⁶ to about 1×10⁻¹³, 1×10⁻¹ to about 1×10⁻¹¹, 1×10⁻² to about 1×10⁻¹¹, 1×10⁻⁴ to about 1×10⁻¹¹, 1×10⁻⁶ to about 1×10⁻¹¹, 1×10⁻¹ to about 1×10⁻⁹, 1×10⁻² to about 1×10⁻⁹, 1×10⁻⁴ to about 1×10⁻⁹, 1×10⁻⁶ to about 1×10⁻⁹, 1×10⁻¹ to about 1×10⁻⁷, 1×10⁻² to about 1×10⁻⁷, 1×10⁻⁴ to about 1×10⁻⁷, 1×10⁻⁶ to about 1×10⁻⁷, 1×10⁻¹ to about 1×10⁻⁵, 1×10⁻² to about 1×10⁻⁵, 1×10⁻⁴ to about 1×10⁻⁵, or 1×10⁻¹ to about 1×10⁻¹³ M.

It will be appreciated by a person skilled in the art that in some situations the method may comprise one or more amplification steps to increase the concentration of the target in a sample to a level that the sensor of the invention is capable of detecting. Suitable methods include, for example, the methods described in US2016/0046977, the entire contents and disclosures of which are hereby incorporated by reference.For example, in some embodiments the amplification reaction may be a polymerase chain reaction (PCR), for example a real-time nucleic acid amplification such as a real-time polymerase chain reaction (RT-PCR),In another aspect the present invention relates to a a method for amplifying a target nucleic acid, and an apparatus and system therefor.

Those skilled in the art will understand that the term “real-time nucleic acid amplification reaction” contemplates the monitoring in real time of the amplification of nucleic acid, for example by elongation catalysed by a nucleic acid polymerase, for example in a polymerase chain reaction.

Those skilled in the art will understand that the terms “real-time polymerase chain reaction”, “real-time PCR”, and “RT-PCR are used interchangeably herein and contemplate the monitoring in real time of the amplification of nucleic acid by the polymerase chain reaction.

A polymerase chain reaction typically comprises repeated steps of annealing a forward and a reverse probe to a target nucleotide or oligonucleotide, elongation, and dissociation.

A polymerase chain reaction typically comprises the repeated steps of annealing a forward and a reverse probe to target nucleic acid, elongation, and dissociation. In one embodiment, the PCR of the methods of the invention involves optionally maintaining the reaction volume or PCR reaction mixture for a period and at a temperature sufficient to allow initial dissociation of the target nucleic acid, followed by repeated cycles of maintaining the reaction volume or the PCR reaction mixture for a period and at a temperature sufficient to allow hybridisation of the target nucleic acid to forward and reverse primers, maintaining the reaction volume or the PCR reaction mixture for a period at a temperature sufficient to allow elongation of the forward and reverse primers by polymerisation, maintaining the reaction volume or the PCR reaction mixture for a period at a temperature sufficient to allow dissociation of the nucleic acid duplex, thereby to provide amplified nucleic acid.

Those skilled in the art will appreciate that the reagents for a nucleic acid amplification reaction will typically include buffers, and nucleotides, particularly nucleotide triphosphates such as dATP, dCTP, dGTP or dTTP.

As will be appreciated by those skilled in the art on reading the specification, target nucleic acid specificity can be readily achieved by appropriate selection of the nucleic acid primers covalently bound to the electrode and present in the reaction mixture. The present invention provides highly sensitive real-time PCR using electrochemical detection and/or measurement in the presence of one or more redox couples, via an electrode comprising electrochemically-active conducting polymer to which is covalently bound one or more nucleic acid primers. The electrode is present in a reaction volume in which the polymerase chain reaction takes place.

In various embodiments, the reaction volume comprises an electrochemical cell, such as a miniature electrochemical cell. In one embodiment, the electrochemical cell comprises a heat source, such as an embedded heater, and two electrodes, for example two printed carbon electrodes. It will be apparent to a person skilled in the art on reading this specification that one of the electrodes is the working (or detection) electrode, comprising the electrochemically-active conducting polymer and surface bound primer, and the other electrode is the reference or counter electrode.

In one embodiment, the reaction volume is in the form of a miniature well, such as that present on a microtitre plate. In one embodiment, the invention provides multiple reaction volumes, for example multiple wells, having a single heater or thermocycler. In one embodiment each reaction volume, for example each well, is individually addressable and may be configured to amplify the same target sequence thereby to achieve redundancy of measurement for improved accuracy, or to amplify a different target sequence thereby to have multiple analyte capability.

In one embodiment, the real-time PCR system comprises multiple reaction volumes provided with a corresponding heating portion and a detector for detecting the impedance of the first electrode. In certain embodiments, each reaction volume is provided with a controller for controlling temperatures of the individual heating regions independently with high accuracy.

In one embodiment, the reaction volumes for PCR reactions are desirably in the form of microcavities. For example, in one embodiment the reaction volume is in the nanolitre volume range, so as to allow for extremely high density arrays of reaction volumes.

In one embodiment the reaction volume is associated with a Peltier element to perform heating, cooling, and/or temperature control.

In one embodiment, the detection electrode is prepared as follows: the electrochemically-active copolymer is prepared in colloidal suspension by chemical oxidation or electroless oxidative polymerisation, optionally in the presence of one or more templating agents to maximize the ratio of surface to volume and control the microstructure in the final deposit through control of size and shape of the colloidal particles. For example, in one embodiment the electrochemically-active conducting polymer comprises one or more nanotubes, nano wires, or similar nano-scale structures. The polymer is separated by centrifugation, and washed then resuspended in buffer. The polymer with attached primer is then deposited onto the carbon working electrode by micropipette or by electrochemical printing.

Other methods for preparing substrates comprising electrochemically-active conducting polymers are well known in the art, and are amenable to use in the preparation of the electrodes of the present invention.

The present invention recognizes that during real time PCR, the composition of the solution steps in a defined way from one cycle to the next. Therefore, signal correlated with a step is derived specifically from the effect of the presence of the nucleic acid target. Since the concentration of target in the solution approximately doubles in each step, the steps are clear and distinct and progress in a well-defined way, and are clearly separable from any general, non-specific drift in the electrochemical properties of the electrode interface. A further advantage is that the high-temperature stage, at 95° C., dissociates nucleic acid from the electrode surface and thus ‘resets’ the surface. Thus, immediately after this step, the surface is in a defined initial condition of un-hybridised primer/probe. Evolution of the signal from this state, and a systematic change from one cycle to the next, provides another specific indicator of the presence of the target nucleic acid. Thus, the present invention allows specific detection and quantification of target nucleic acid in a small number of cycles —that is, in a time that is significantly shorter than that required for detection and quantification using other methods, such as optical fluorescence methods.

It will be appreciated by those skilled in the art on reading this specification that the electrochemical and mechanical stability of the electrochemical measurement interface upon cycling to the high temperatures necessary to implement PCR is important. In particular, for methods utilizing conducting polymers, irregular or otherwise large changes in adhesion of the polymer to the electrode substrate, or in polymer microstructure, or in state of oxidation or doping of the polymer, is undesirable as it may cause changes in electrochemical reaction rate at the polymer-solution interface that militates against reliable and quantitative measurement.

The following non-limiting examples are provided to illustrate the present invention and in no way limit the scope thereof.

### EXAMPLES

Materials and Methods

All reagents for the synthesis of the compounds described in this section were sourced from AK Scientific, Sigma Aldrich, Alpha DNA or other commercial sources and were used as received.

All reactions were carried out under an atmosphere of nitrogen and at room temperature unless otherwise stated.

Solvents were dried prior to use.

Nuclear magnetic resonance was carried out on a Bruker 300 MHz or 400 MHz instrument. Samples were prepared in CDCl₃, DMSO-d₆ or CD₃OD. All melting points were measured using a Reicher-Kofler block and are uncorrected.

Cyclic Voltammetry

Polymers formed either by chemical polymerisation or electropolymerisation were characterised using cyclic voltammetry, UV-visible spectroscopy and by measurements of conductivity.

Cyclic voltammetry was used to evaluate the potential at which polymerisation occurs and to investigate the electroactivity of the polymers formed. Unless indicated otherwise, in the case of polymerisation, CV was performed in the DMF/PBS (1:1) containing the 1:50 ratio of monomers 60 (ThPhON) and 7 (ThPhEG) or monomer 50 (PyPhON) and pyrrole and 0.1M sodium p toluenesulfonate (NaTos). For the characterisation of electroactivity, CV experiments were performed in PBS (pH: 7.4).

Ultraviolet-Visible Spectroscopy

UV-Visible spectroscopy was carried out using a Shimadzu Spectrophotometer (Model UV-1700) through the dissolution of the polymer in CH₂Cl₂. The polymer solution was diluted to an absorbance value below 0.05 to prevent self-quenching.

Conductivity Measurements

Conductivity was measured using a Jandel 4-pin probe. 250 mg of polymer was pressed into a tablet and conductivity was measured by pressing the 4-point probe into the pellet, unless indicated otherwise.

DNA Sensing Experiments

Materials and Methods

The reagents and solvents used in the experiments described in this section were purchased from Sigma Aldrich, J T Baker, Alpha DNA or other commercial sources and were used as received unless stated otherwise.

DNA sequences as shown in Table 3 were obtained from Alpha DNA.

Electrodes

Unless indicated otherwise, 1.6 mm diameter gold disk (MF-2014), 1.6 mm diameter Platinum disk (MF-2013), 3 mm diameter glassy carbon (GC) (MF-2012), standard Ag/AgCl (MF-2052) and platinum (Pt) wire electrodes were purchased from BASI. Leak-free Ag/AgCl (ET072) electrode was purchased from Warner Instruments. Gold, GC and Pt electrodes were employed as working electrodes (WE) whereas standard Ag/AgCl and leak-free electrodes were used as the reference electrodes (RE) and Pt wire was used as the counter electrode (CE). Pt, gold and GC electrodes were polished before use using 0.5 μm alumina slurry and were then ultra-sonicated in ethanol and deionized water (Milli-Q) for 5 minutes each. Screen printed carbon electrodes were from DropSense (type DRP150).

Buffer preparation

PBS buffer was prepared by dissolving phosphate buffered saline tablets in 200 mL of deionised water (Milli-Q, 18.2 MΩ·cm (at 25° C.)), stored in the freezer at −18° C. and degassed for 10 minutes before use.

Instruments

For electropolymerisation in example 7 a CH 650 potentiostat, for electrochemical impedance measurements, and for polymerisation experiments in other examples unless indicated otherwise, a Bio-Logic bio-potentiostat was employed. 1.6 mm

Platinum disk electrode (BASI), 1.6 mm Au disk electrode (BASI), 3 mm glassy carbon electrode (BASI), as working (WE), Ag/AgCl (in 3M KCl) and leak free (Warner Instruments) as reference electrodes and a platinum spiral wire was employed as the counter (CE) electrode respectively.

### 1.1 Exampe 1

As shown in Scheme 1, this example describes the synthesis of a 2,2′-(2,5-bis(2-(2-(2-methoxyethoxy)ethoxy)ethoxy)-1,4-phenylene)dithiophene (TGThP) monomer 7.

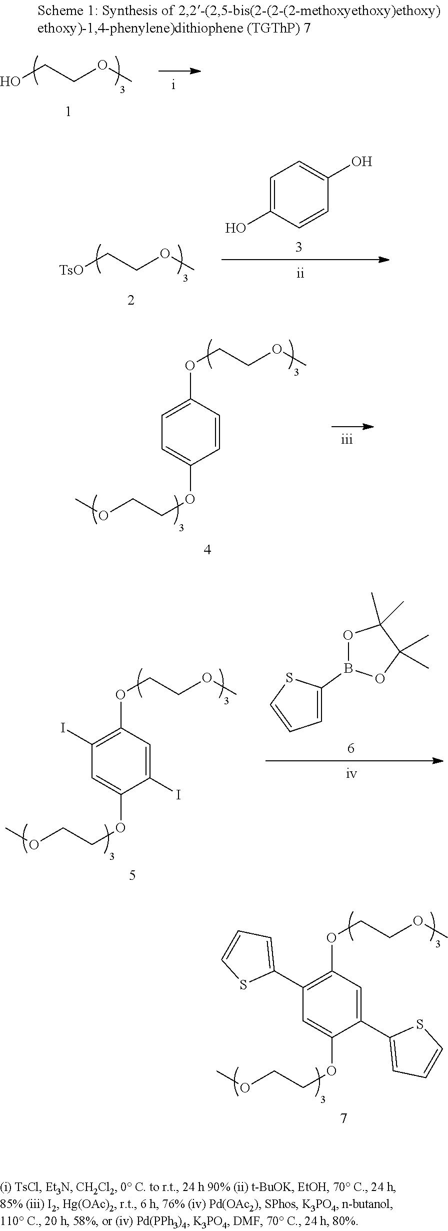

### 2-(2-(2-Methoxyethoxy)ethoxy)ethyl 4-methylbenzenesulfonate 2

To a stirred solution of 2-(2-(2-methoxyethoxy)ethoxy)ethanol 1 (2.00 g, 12.0 mmol) and tosyl chloride (2.29 g, 10.94 mmol) in CH₂Cl₂ (10 mL) under an atmosphere of nitrogen at 0° C., Et₃N (3.06 mL, 21.9 mmol) was added dropwise. The solution was warmed to room temperature and stirred for 18 h. The reaction was then quenched with the addition of water (10 mL) and the organic layer separated. The aqueous phase was extracted with CH₂Cl₂ (3×20 mL) and the combined organic extracts washed with brine (10 mL), dried (MgSO₄) and solvent was removed in vacuo. The crude product was purified by flash chromatography (3:1, ethyl acetate, hexanes) to yield title product 2 (2.630 g, 86%) as a red oil. R_{F}=0.2, 3:1 ethyl acetate, hexanes. ¹H NMR (400 MHz; CDCl₃) 3.37 (3H, s, OCH₃), 3.51-3.70 (10H, m, OCH₂), 3.59 (3H, s, Ar—CH₃), 4.14-4.19 (2H, m, OCH₂), 7.36 (2H, d, J=8.0 Hz, 3-H and 5-H), 7.79 (2H, d, J=8.0 Hz, 2-H and 6-H); ¹³C NMR (100 MHz; CDCl₃) 21.6, 59.6, 68.7, 69.2, 70.5, 70.6, 70.8, 71.9, 128.0, 129.8, 133.1, 144.8. The ¹H NMR and ¹³C NMR are in agreement with literature.¹

### 1,4-Bis(2-(2-(2-methoxyethoxy)ethoxy)ethoxy)benzene 4

To a stirred solution of 2-(2-(2-methoxyethoxy)ethoxy)ethyl 4-methylbenzenesulfonate 2 (2.86 g, 9.00 mmol) and hydroquinone 3 (0.330 g, 3.00 mmol) in ethanol (30 mL) at 0° C., t-BuOK (1.01 g, 9.00 mmol) was added and the resulting mixture was heated at 70° C. for 24 h. The reaction mixture was then cooled to room temperature and quenched with water (10 mL) and extracted with CH₂Cl₂ (3×30 mL). The combined extracts were washed with brine (30 mL), dried (Na₂SO₄) and the solvent removed in vacuo. The crude product was purified using flash chromatography (3:1, ethyl acetate, hexanes) to yield title product 4 (0.591 g, 49%) as a red oil. R_{F}=0.5, (2:1 ethyl acetate, hexanes), ¹H NMR (400 MHz; CDCl₃) 3.38 (6H, s, OCH₃), 3.56-3.59 (4H, m, OCH₂), 3.69-3.73 (8H, m, OCH₂), 3.74-3.76 (4H, m, OCH₂), 3.79-3.83 (4H, m, OCH₂), 4.02-4.08 (4H, m, OCH₂), 6.82 (4H, s, Ar—H); ¹³C NMR (100 MHz; CDCl₃) 59.0 (CH₃), 66.0 (OCH₂), 68.0 (OCH₂), 69.7 (OCH₂), 70.4 (OCH₂), 70.6 (OCH₂), 71.9 (OCH₂), 115.6 (CH), 153.0 (C-1 and C-4). The ¹H NMR and ¹³C NMR were in agreement with literature values.²

### 1,4-Diiodo-2,5-bis(2-(2-(2-methoxyethoxy)ethoxy)ethoxy)benzene 5

To a stirred solution of 1,4-bis(2-(2-(2-methoxyethoxy)ethoxy)ethoxy)benzene 4 (0.71 g, 1.77 mmol) and iodine (1.79 g, 7.09 mmol) in CH₂Cl₂ (20 mL), mercury acetate (2.26 g, 7.09 mmol) was added and the resulting solution was stirred at room temperature for 5 h. The mixture was then diluted with CH₂Cl₂ (20 mL) and filtered through celite. The organic extract was washed with sat. aq. Na₂S₂O₃ (20 mL), sat. aq. NaHCO₃ (20 mL), water (20 mL) and brine (20 mL). The organic extract was dried (Na₂SO₄) and the solvent was removed in vacuo to yield title product 5 (0.782 g, 76%) as an orange oil, which was used without further purification. R_{F}=0.5, 3:1 ethyl acetate, hexanes, ¹H NMR (400 MHz; CDCl₃) 3.38 (6H, s, OCH₃), 3.55-3.57 (4H, m, OCH₂), 3.62-3.66 (4H, m, OCH₂), 3.67-3.71 (4H, m, OCH₂), 3.77-3.80 (4H, m, OCH₂), 3.87 (4H, t, J=4.6 Hz, CH₂), 4.10 (4H, t, J=4.6 Hz, CH₂), 7.23 (2H, s, Ar—H); ¹³C NMR (100 MHz; CDCl₃) 59.1, 69.6, 70.3, 70.5, 70.6, 70.8, 71.2, 86.4, 123.6, 153.1. The ¹H and ¹³C NMR data were in agreement with literature values.³

### 2,2′-(2,5-Bis(2-(2-(2-methoxyethoxy)ethoxy)ethoxy)-1,4-phenylene)dithiophene (TGThP) 7

Compound 7 was prepared by two procedures—A and B.

Procedure A

To a stirred solution of 1,4-diiodo-2,5-bis(2-(2-(2-methoxyethoxy)ethoxy)ethoxy)benzene 5(0.300 g, 0.416 mmol) in n-butanol (3 mL) thiophene boronate 6 (0.231 g, 1.10 mmol), Pd(OAc)2 (0.010 g, 4.46 μmol), SPhos (0.038 g, 0.093 mmol) and K3PO₄ (0.292 g, 1.38 mmol) was added. The mixture was placed under an atmosphere of nitrogen, degassed by freeze-thaw-cycling, sealed and heated at 110° C. in a pressure tube for 20 h. The resulting mixture was then cooled to room temperature, diluted with CH₂Cl₂ (10 mL), filtered through a silica plug and the solvent removed in vacuo. The crude product was purified by flash chromatography (3:1, ethyl acetate, hexanes) to yield title product 7 (0.151 g, 58%) as a brown oil. R_{F}=0.4 (2:1 ethyl acetate, hexane), IR vₘₐₓ (neat)/cm⁻¹ 3671, 2972, 2882, 1453, 1353, 1102; ¹H NMR (400 MHz; CDCl₃) 3.36 (6H, s, OCH₃), 3.53 (4H, m, OCH₂), 3.64 (4H, m, OCH₂), 3.69 (4H, m, OCH₂), 3.75 (3H, m, OCH₂), 3.93 (OCH₂), 4.24 (4H, m, OCH₂), 7.09 (2H, dd, J=5.1, 3.6 Hz, 4-H), 7.28 (2H, s, 3′-H and 6′-H), 7.32 (2H, dd, J=3.6, 1.2 Hz, 5-H), 7.55 (2H, dd, J=5.1, 1.2 Hz, 3-H); ¹³C NMR (100 MHz; CDCl₃) 59.0 (CH₃), 69.2 (OCH₂), 69.8 (OCH₂), 70.6 (OCH₂), 70.7 (OCH₂), 70.9 (OCH₂), 71.9 (ArOCH₂), 113.67 (C-3′ and C-6′), 123.4 (C-2′ and C-5′), 125.6 (C-5), 125.7 (C-3), 126.9 (C-4), 139.0 (C-2), 149.4 (C-1′ and C-4′). HRMS (EI) found (MK⁺) 605.1649. C₂₈H₃₈KO₈S₂ requires 605.1640.

Procedure B

To a solution of 1,4-diiodo-2,5-bis(2-(2-(2-methoxyethoxy)ethoxy)ethoxy)benzene 5(0.670 g, 1.02 mmol) in DMF (20 mL), thiophene boronate 6 (0.492 g, 2.35 mmol), K₃PO₄ (0.719 g, 3.06 mmol), Pd(PPh₃)₄ (0.011 g, 0.010 mmol) was added and the mixture was placed under an atmosphere of N₂. The mixture was heated at 70° C. for 24 h, cooled to r.t., quenched with water (20 mL) and extracted with CH₂Cl₂ (40 mL). The organic extract was then washed with water (3×30 mL), brine (20 mL), dried (MgSO₄) and solvent removed in vacuo. The crude product was purified by flash chromatography (1:1, hexanes, ethyl acetate) to yield title product 7 (0.461 g, 80%) as a yellow solid.

### 1.2 Example 2

As shown in Scheme 2, this example describes the synthesis of 2,2′-(2,5-dimethoxy-1,4-phenylene)dithiophene (MeThP) monomer 10.

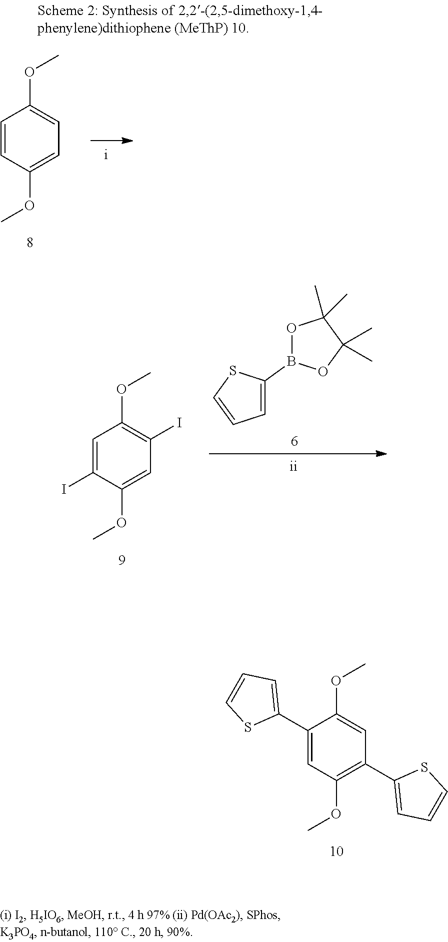

### 1,4-Diiodo-2,5-dimethoxybenzene 9

Following the procedure by Ko et al,³ a solution of H₅IO₆ (2.92 g, 12.5 mmol) in methanol (25 mL) was stirred for 10 min, then iodine (6.38 g, 25.0 mmol) was added to the mixture. 1,4-Dimethoxybenzene 8 (2.70 g, 20.0 mmol) was added and the mixture was then heated at 70° C. for 4 h. The mixture was poured into a solution of Na₅S₂O₃ (5.00 g, 31.6 mmol) in water (50 mL). The solution was filtered and the precipitate was washed with methanol (20 mL), redissolved in CH₂Cl₂ (20 mL) and filtered through a sintered funnel and the filtrate was evaporated in vacuo to afford title product 9 as a white solid (7.52 g, 97%). ¹H NMR (400 MHz; CDCl₃) 3.82 (6H, s, CH₃), 7.19 (2H, s, Ar). The ¹H NMR data was in agreement with literature values.⁴

### 2,2′-(2,5-Dimethoxy-1,4-phenylene)dithiophene (MeThP) 10

To a stirred solution of 1,4-diiodo-2,5-dimethoxybenzene 9 (0.16 g, 0.416 mmol) in n-butanol (3 mL), thiophene boronate 6 (0.23 g, 1.10 mmol), Pd(OAc)₂ (0.010 mg, 4.46 μmol), SPhos (0.038 g, 0.093 mmol) and K₃PO₄ (0.29 g, 1.38 mmol) was added. The mixture was placed under an atmosphere of nitrogen, degassed by freeze-thaw-cycling, sealed and heated at 110° C. in a pressure tube for 20 h. The resulting mixture was then cooled, diluted with CH₂Cl₂ (10 mL), filtered through a silica plug and the solvent removed in vacuo. The crude product was purified by flash chromatography (3:1, ethyl acetate, hexanes) to yield title product 10 (0.54 g, 90%) as a yellow solid. MP=58-60° C., R_{f}=0.7 (3:1, hexanes ethyl acetate), IR vₘₐₓ (neat)/cm⁻¹ 3340, 3093, 2993, 2939, 2829, 1533, 1393, 1289, 1039; ¹H NMR (400 MHz; CDCl₃); 3.93 (6H, s, OCH₃), 7.10 (2H, dd, J=5.1, 4.1 Hz, 4-H), 7.25 (2H, s, 3′-H and 6′-H), 7.33 (2H, dd, J=4.1, 1.1 Hz, 5-H), 7.35 (2H, dd, J=5.1, 1.1 Hz, 3-H); ¹³C NMR (100 MHz; CDCl₃) 36.5 (CH₃O), 112.0 (C-3′ and C-6′), 123.0 (C-2′ and C-5′), 125.5 (C-5), 125.7 (C-3), 126.9 (C-4), 139.0 (C-2), 150.0 (C-1′ and C-4′). HRMS (EI) found (MH⁺) 303.0497. C₁₆H₁₅O₂S₂ requires 303.0508.

### 1.3 Example 3

As shown in Scheme 3, this example describes the synthesis of 6,6′-((2,5-di(thiophen-2-yl)-1,4-phenylene)bis(oxy))dihexanoic acid 22, a monomer comprising two carboxylic acid groups.

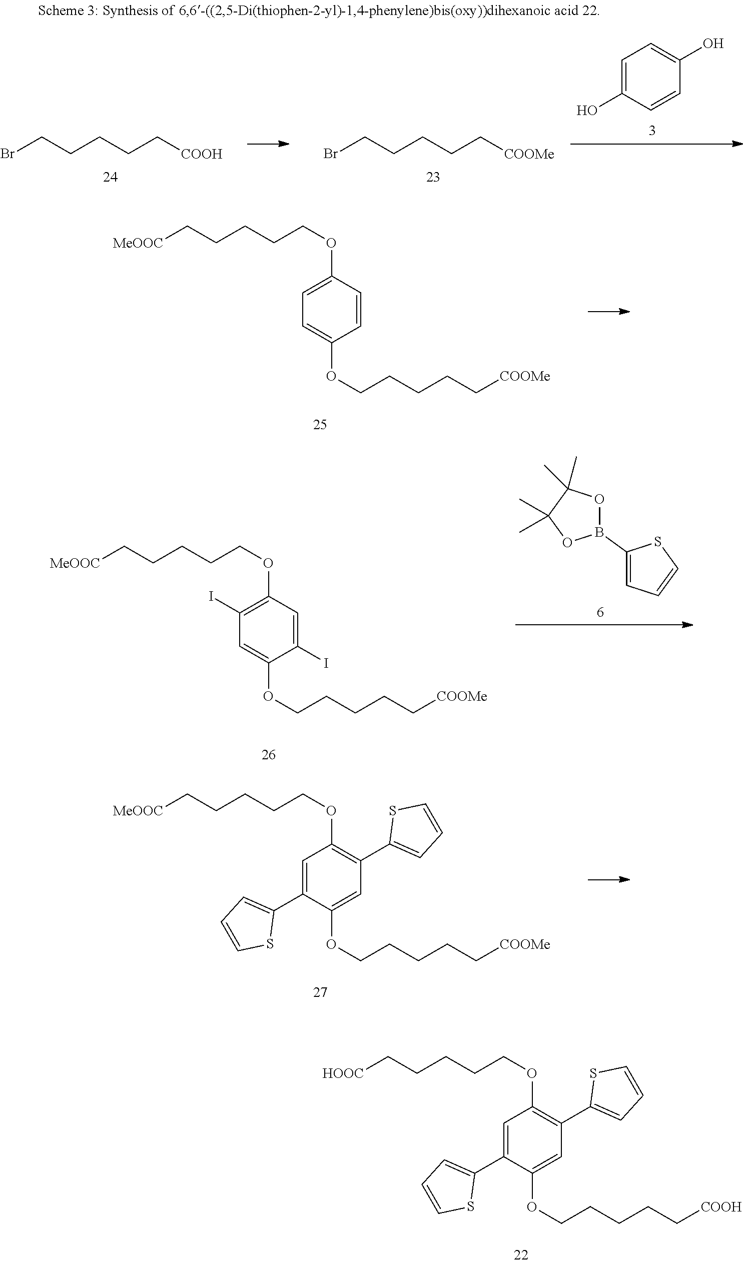

### Methyl 6-bromohexanoate 23

To a stirred solution of 6-bromohexanoic acid 24 (5.00 g, 25.6 mmol) in methanol (75 mL) at 0° C., thionyl chloride (1.86 mL, 25.6 mmol) was added dropwise. The mixture was warmed to room temperature and stirred for 24 h. The solvent was removed in vacuo and the residue was dissolved in ethyl acetate (20 mL), washed with water (2×10 mL), sat. NaHCO₃ (10 mL), brine (10 mL) and dried (MgSO₄). The solvent was removed in vacuo to give the title product 23 (5.13 g, 96%) as a yellow oil which was used without further purification. ¹H NMR (300 MHz; CDCl₃) 1.42-1.52 (2H, m, CH₂), 1.56-1.66 (2H, m, CH₂), 1.85-1.90 (2H, m, CH₂), 2.33 (2H, t, J=3.0 Hz, CH₂), 3.41 (2H, t, J=6.0 Hz, CH₂), 3.67 (3H, s, CH₃). The ¹H values are in agreement with literature.⁵

### Dimethyl 6,6′-(1,4-phenylenebis(oxy))dihexanoate 25

Potassium hydroxide (1.00 g, 17.8 mmol) was added to a solution of 1,4-hydroquinone 3 (0.650 g, 5.90 mmol) and methyl 6-bromohexanoate 23 (3.23 g, 15.3 mmol) in DMSO (10 ml). The mixture was stirred at room temperature for 24 h and then quenched with water (50 mL), extracted with CH₂Cl₂ (30 mL), dried (MgSO₄) and the solvent was removed in vacuo. The crude product was recrystallized from hexane to give the title compound 25 (3.76 g, 90%) as an off-white solid. ¹H NMR (300 MHz; CDCl₃) 1.80-1.49 (12H, m, CH₂), 2.39 (4H, t, J=6.0 Hz, CH₂CO₂Me), 3.67 (6H, s, OCH₃), 3.90 (4H, t, J=6.0 Hz, OCH₂), 6.80 (2H, s, Ar—H). The ¹H values are in agreement with literature.²

### Dimethyl 6,6′-((2,5-diiodo-1,4-phenylene)bis(oxy))dihexanoate 26

A solution of iodine monochloride (3.20 g, 19.7 mmol) in methanol (10 mL) was added dropwise to a stirred solution of dimethl 6,6′-(1,4-phenylenebis(oxy))dihexanoate 25 (2.00 g, 5.46 mmol) in methanol (20 mL) at 0° C. The mixture was heated at reflux for 4 h and left to cool to room temperature. The precipitate was collected, washed with cold methanol to yield title product 26 (3.24 g 96%) as pale yellow crystals. ¹H NMR (300 MHz; CDCl₃) 1.59-1.80 (12H, m, CH₂), 2.39 (4H, t, J=6.0 Hz, CH₂CO₂Me), 3.71 (6H, s, OCH₃), 3.99 (4H, t, J=6.0 Hz, OCH₂), 7.20 (2H, s, Ar—H). The ¹H NMR data was in agreement with literature values.⁶

### Dimethyl 6,6′-((2,5-di(thiophen-2-yl)-1,4-phenylene)bis(oxy))dihexanoate 27

A stirred solution of dimethyl 6,6′-((2,5-diiodo-1,4-phenylene)bis(oxy))dihexanoate 26 (1.00 g, 1.68 mmol), thiophene boronate 6 (0.747 g, 3.05 mmol), tripotassium phosphate (1.18 g, 5.04 mmol), and tetrakis(triphenylphosphine)palladium(0) (0.185 g, 0.168 mmol) in DMF under an atmosphere of nitrogen was heated at 70° C. for 24 h. The mixture was then cooled, quenched with water (20 mL) and extracted with CH₂Cl₂ (2×20 mL). The combined organic extracts were washed with brine (20 mL), dried (MgSO₄) and the solvent was removed in vacuo. The crude product was purified using flash chromatography (4:1, hexanes ethyl acetate) to yield title product 27 (0.624 g, 70%) as a yellow solid. R_{F}=0.5 (3:1 hexanes, ethyl acetate); IR vₘₐₓ (neat)/cm⁻¹ 2947, 2862, 1728, 1393, 1216; ¹H NMR (400 MHz; CDCl₃) 1.55-1.65 (4H, m, CH₂), 1.69-1.77 (4H, m, CH₂), 1.88-1.95 (4H, m, CH₂), 2.36 (4H, t, J=7.4 Hz, CH₂), 3.67 (6H, s, CH₃), 4.08 (4H, t, J=6.1 Hz, CH₂), 7.09 (2H, dd, J=5.5, 3.7 Hz, 4-H), 7.24 (2H, s, 3′-H), 7.43 (2H, dd, J=5.5, 1.1 Hz, 3-H), 7.51 (2H, dd, J=3.7, 1.1 Hz, 5-H); ¹³C NMR (100 MHz; CDCl₃) 24.7 (CH₂), 25.9 (CH₂), 29.1 (CH₂), 34.0 (CH₂), 51.5 (CH₃), 69.4 (OCH₂), 112.9 (C-2′), 113.9 (C-3′), 123.1 (C-3), 125.2 (C-5), 125.8 (C-4), 139.2 (C-2), 149.2 (C-1′), 174.1 (C═O); HRMS (EI) found (M⁺) 531.1863. C₂₈H₃₅O₆S₂ requires 531.1870.

### 6,6′-((2,5-Di(thiophen-2-yl)-1,4-phenylene)bis(oxy))dihexanoic acid 22

To a stirred solution of dimethyl 6,6′-((2,5-di(thiophen-2-yl)-1,4-phenylene)bis(oxy))dihexanoate 27 (0.419 g, 0.79 mmol) in THF (30 mL) and MeOH (30 mL), 4M aq. NaOH (20 mL) was added and the resulting solution was stirred at room temperature for 3 h. The solvent was removed in vacuo and the crude product was re-dissolved in water (20 mL). The mixture was acidified with 2M HCl until a precipitate was formed and the precipitate was collected via vacuum filtration to yield title product 22 (0.355 g, 90%) as a yellow solid, which was used without further purification. ¹H NMR (400 MHz; DMSO-_{d}6) 1.53-1.56 (4H, m, CH₂), 1.57-1.64 (4H, m, CH₂), 1.83-1.87 (4H, m, CH₂), 2.20-2.26 (4H, m CH₂), 4.12-4.17 (4H, m, CH₂), 7.14 (2H, dd, J=4.9, 3.9 Hz, 4-H), 7.43 (2H, s, 3′-H), 7.57 (2H, d, J=4.9 Hz, 3-H), 7.71 (2H, d, J=3.9 Hz, 5-H) 12.00 (2H, s, COOH);).; ¹³C NMR (100 MHz; DMSO-d₆) 24.3 (C-4′), 25.3 (C-3′), 28.6 (C-2′), 35.0 (C-5′), 69.0 (C-1′), 112.0 (C-3′), 112.1 (C-2′), 125.5 (C-3), 126.7 (C-5), 126.8 (C-4), 138.0 (C-2), 148.9 (C-1′), 174.4 (COOH); IR vₘₐₓ (neat)/cm⁻¹ 2943, 2861, 2623, 1706, 1493, 1217; HRMS (EI) found (M⁺) 525.1370. C₂₆H₃₀NaO₆S₂ requires 525.1376.

### 1.4 EXAMPLE 4

As shown in Scheme 4 the following example describes the synthesis of 2,2′-(2,5-dimethoxy-1,4-phenylene)bis(1H-pyrrole) 28

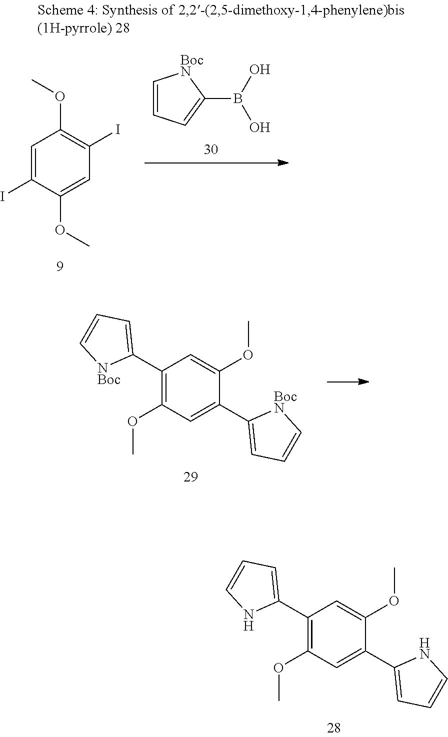

### Di-tert-butyl 2,2′-(2,5-dimethoxy-1,4-phenylene)bis(1H-pyrrole-1-carboxylate) 29

A stirred solution of 1,4-diiodo-2,5-dimethoxybenzene 9 (3.34 g, 7.43 mmol), boronate 30 (3.45 g, 16.4 mmol), tripotassium phosphate and tetrakis(triphenylphosphine)palladium(0) (0.410 g, 0.372 mmol) in DMF (30 mL) under at atmosphere of nitrogen was stirred at 70° C. for 24 h. The mixture was then cooled, quenched with water (20 mL) and extracted with CH₂Cl₂ (3×10 mL), washed with brine (20 mL), dried (MgSO₄) and the solvent was removed in vacuo. The crude product was purified using flash chromatography (3:1, hexanes, ethyl acetate) to yield title product 29 (1.55 g, 40%) as a red solid. MP=134-136° C.; R_{F}=0.2 (2:1 ethyl acetate, hexanes), IR vₘₐₓ (neat)/cm⁻¹ 3442, 3311, 2978, 1732, 1325; ¹H NMR (400 MHz; CDCl₃) 1.34 (18H, s, (CH₃)₃C), 3.71 (6H, OCH₃), 6.17 (2H, dd, J=3.1, 1.6 Hz, 4-H), 6.24 (2H, t, J=3.1 Hz, 3-H), 6.81 (2H, s, 3′-H), 7.35 (2H, dd, J=3.1, 1.8 Hz, 5-H); ¹³C NMR (100 MHz; CDCl₃) 27.6 ((CH₃)₃C), 56.0 (OCH₃), 83.1 (C(CH₃)₃), 110.3 (C-3), 112.9 (C-4), 114.0 (C-3′), 122.0 (C-5), 123.7 (C-2′), 131.1 (C-1), 149.3 (C═O), 150.1 (C-1′).

The ¹H and ¹³C NMR values are in agreement with literature.⁷

### 2,2′-(2,5-Dimethoxy-1,4-phenylene)bis(1H-pyrrole) 28

Sodium (0.635 g, 4.30 mmol) was dissolved in methanol (5 mL) and the resulting solution was added to a solution of di-tert-butyl 2,2′-(2,5-dimethoxy-1,4-phenylene)bis(1H-pyrrole-1-carboxylate) 29 (0.101 g, 0.216 mmol) in THF (5 mL). The mixture was stirred at room temperature for 24 h, quenched with water (10 mL) and extracted with ethyl acetate (3×5 mL), dried and the solvent was removed in vacuo. The crude product was purified with flash chromatography (3:1 hexanes, ethyl acetate) to yield title product 28 (0.40 g, 71%) as a white solid. ¹H NMR (400 MHz; DMSO-_{d}6) 3.89 (6H, s CH₃), 6.10-6.12 (2H, m, 4-H), 6.62-6.64 (2H, m, 3-H), 6.82-6.84 (2H, m, 5-H), 7.25 (2H, s, 3-H′), 11.0 (2H, s, 1-H); ¹³C NMR (100 MHz; DMSO-_{d}6) 56.0 (CH₃), 108.2 (C-3), 108.3 (C-4), 109.7 (C-3′), 118.5 (C-5), 119.1 (C-2′), 127.9 (C-1), 149.1 (C-1′). The ¹H and ¹³C NMR values are in agreement with literature.⁷

### 1.5 Example 5

As shown in Scheme 5 this example describes the synthesis of 2,2′-(2,5-bis(2-(2-(2-methoxyethoxy)ethoxy)ethoxy)-1,4-phenylene)bis(1H-pyrrole) 33.

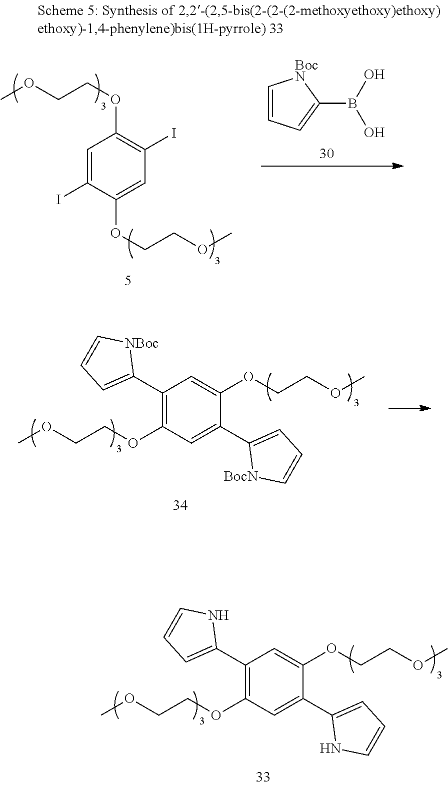

### Di-tert-butyl 2,2′-(2,5-bis(2-(2-(2-methoxyethoxy)ethoxy)ethoxy)-1,4-phenylene)bis(1H-pyrrole-1-carboxylate) 34

To a stirred solution of 1,4-diiodo-2,5-bis(2-(2-(2-methoxyethoxy)ethoxy)ethoxy)benzene 5 (0.300 g, 0.416 mmol) in n-butanol (5 mL), pyrrole boronic acid 30 (0.232 g, 1.10 mmol), Pd(OAc)₂ (0.005 g, 0.09 mmol), SPhos (0.038 g, 0.090 mmol) and tripotassium phosphate (0.292 g, 1.38 mmol) were added. The resulting solution was placed under an atmosphere of nitrogen, degassed by freeze-thaw-cycling, sealed and heated at 80° C. in a pressure chamber for 24 h. The resulting mixture was diluted with CH₂Cl₂ (10 mL) and filtered through a silica plug then purified by flash chromatography (3:1, ethyl acetate, hexanes) to yield the protected pyrrole product 34 (0.243g, 80%) as a green oil.

R_{f}=0.4 (4:1, ethyl acetate, hexanes); IR vₘₐₓ (neat)/cm⁻¹ 2876, 1488, 1430, 1350, 1200, 1100, 1070; ¹H NMR (400 MHz; CDCl₃) 1.37 (18H, s, (CH₃)₃C), 3.36 (6H, s, CH₃), 3.54-3.72 (8H, m, CH₂), 3.88-3.92 (4H, m, CH₂), 3.95-3.98 (4H, m CH₂), 3.99-4.03 (4H, m, CH₂), 4.19 (4H, m, CH₂), 6.25-6.28 (2H, m, 3-H), 6.57-6.59 (2H, m, 4-H), 6.88-6.89 (2H, m, 5-H), 7.15 (2H, s, 3′ -H); ¹³C NMR (100 MHz; CDCl₃) 27.8 ((CH₃)3C), 59.0 (CH₃), 69.2 (CH₂), 69.8 (CH₂), 70.6 (CH₂), 70.7 (CH₂), 70.9 (CH₂), 71.9 (CH₂), 78.0 (C(CH₃)₃), 113.7 (C-3′), 123.4 (C-3), 125.6 (C-4), 126.4 (C-2) 126.9 (C-5) 139.0 (C-2′), 149.4 (C═O), 150.4 (C-1′). HRMS (EI) found (MH⁺) 733.3917. C₃₈H₅₆N₂O₁₂ requires 733.3912.

### 2,2′-(2,5-bis(2-(2-(2-methoxyethoxy)ethoxy)ethoxy)-1,4-phenylene)bis(1H-pyrrole) 33

A solution of protected pyrrole 34 was dissolved in THF (5 mL) and added to a solution of sodium (0.191 g, 8.32 mmol) dissolved in methanol (5 mL). The mixture was stirred at room temperature for 24 h, quenched with water (20 mL) and extracted with ethyl acetate (3×15 mL). The combined extracts were dried (MgSO₄) and the solvent was removed in vacuo. The crude product was purified with flash chromatography (2:1 ethyl acetate, hexanes) to yield title product 33 (0.089 g, 40%) as a red oil. R_{F}=0.4 (2:1 ethyl acetate hexanes), IR vₘₐₓ (neat)/cm⁻¹ 3380, 2873, 1448, 1200, 1100; ¹H NMR (400 MHz; CDCl₃) 3.36 (6H, s, OCH₃), 3.53 (4H, m, OCH₂), 3.65 (4H, m, OCH₂), 3.73 (4H, m, OCH₂), 3.77 (4H, m, OCH₂), 3.92 (4H, m, OCH₂), 4.26 (4H, m, OCH₂), 6.25 (2H, m, 3-H), 6.54 (2H, m, 4-H), 6.87 (2H, m, 5-H), 7.21 (2H, s, 3′-H and 6′-H), 10.47 (2H, s, NH); ¹³C NMR (100 MHz; CDCl₃) 59.2 (OCH₃), 68.7 (OCH₂), 69.6 (OCH₂), 70.5 (OCH₂), 70.6 (OCH₂), 70.7 (OCH₂), 71.9 (OCH₂), 105.8 (C-1), 108.4 (C-3), 112.6 (C-3′ and C-6′), 118.6 (C-4), 120.5 (C-2), 129.4 (C-2′ and C-5′), 149.0 (C-1′ and C-4′). HRMS (EI) found (MNa⁺) 555.2663. C₂₈H₄₀N₂NaO₈ requires 555.2677.

### 1.6 Example 6

As shown in Scheme 6 the following example describes the synthesis of 6,6′-((2,5-Di(1H-pyrrol-2-yl)-1,4-phenylene)bis(oxy))dihexanoic acid 38.

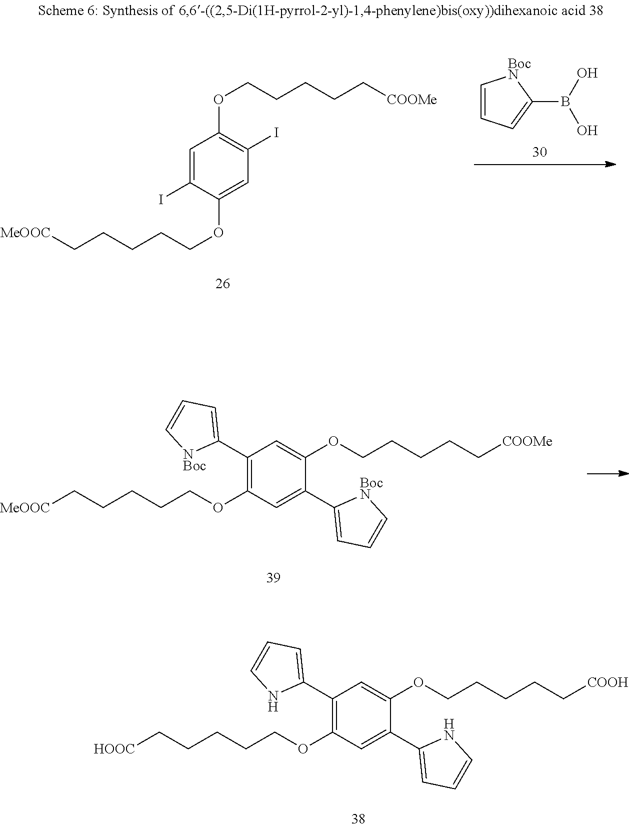 [SMILES: COC(=O)CCCCCOc1cc(I)c(OCCCCCC(=O)OC)cc1I ; CC(C)(C)OC(=O)n1cccc1B(O)O ; COC(=O)CCCCCOc1cc(-c2cccn2C(=O)OC(C)(C)C)c(OCCCCCC(=O)OC)cc1-c1cccn1C(=O)OC(C)(C)C ; O=C(O)CCCCCOc1cc(-c2ccc[nH]2)c(OCCCCCC(=O)O)cc1-c1ccc[nH]1]

### Di-tert-butyl 2,2′-(2,5-bis((6-methoxy-6-oxohexyl)oxy)-1,4-phenylene)bis(1H-pyrrole-1-carboxylate) 39

A stirred solution of dimethyl 6,6′-((2,5-diiodo-1,4-phenylene)bis(oxy))dihexanoate 26 (0.492 g, 0.797 mmol), boronic acid 30 (0.370 g, 1.71 mmol), tetrakis(triphenylphosphine)palladium(0) (0.088 g, 0.080 mmol), K₃PO₄ (0.561 g, 2.39 mmol) in DMF (10 mL) was placed under an atmosphere of nitrogen and heated at 70° C. for 24 h. The mixture was then cooled to room temperature, quenched with water (10 mL) and extracted with CH₂Cl₂ (3×10 mL). The combined organic extracts were washed with brine (10 mL), dried (MgSO₄) and the solvent was removed in vacuo. The crude product was then purified using flash chromatography (4:1, hexanes, ethyl acetate) to yield title product 39 (0.313 g, 56%) as red oil. R_{F}=0.3 (4:1 hexanes, ethyl acetate), IR vₘₐₓ (neat)/cm⁻¹ s 2946, 2868, 1773, 1459, 1331, 1142; ¹H NMR (400 MHz; CDCl₃) 1.33 (18H, s, (CH₃)₃C) 1.50-1.62 (4H, m, CH₂), 1.67-1.75 (4H, m, CH₂), 1.78-1.87 (4H, m, CH₂), 2.23-2.27 (4H, m, CH₂), 3.67 (6H, s, CH₃), 3.75-3.79 (4H, m, CH₂), 6.12 (2H, dd, J=3.4, 2.1 Hz, 4-H), 6.20 (2H, t, J=3.4 Hz, 3-H), 6.77 (2H, s, 3′-H), 7.33 (2H, dd, J=3.4, 2.1 Hz, 5-H); ¹³C NMR (100 MHz; CDCl₃) 24.5 (CH₂), 24.6 (CH₂), 27.7 ((CH₃)₃C) 28.9 (CH₂), 37.8 (CH₃), 69.4 (CH₂), 83.1 (C(CH₃)₃), 110.3 (C-3,) 114.1 (C-4), 114.5 (C-5), 122.1 (C-3′), 126.0 (C-2′), 130.3 (C-1), 149.2 (C═O), 152.0 (C-1′), 174.0 (COOMe). HRMS (EI) found (MNa⁺) 719.3509. C₃₈H₅₂N₂Na₁₀ requires 719.351.

### 6,6′-((2,5-Di(1H-pyrrol-2-yl)-1,4-phenylene)bis(oxy))dihexanoic acid 38

Sodium (0.635 g, 4.30 mmol) was dissolved in methanol (5 mL) and the resulting solution was added to a solution of di-tert-butyl 2,2′-(2,5-bis((6-methoxy-6-oxohexyl)oxy)-1,4-phenylene)bis(1H-pyrrole-1-carboxylate) 39 (101 mg, 0.216 mmol) in THF (5 mL). The mixture was stirred at room temperature for 24 h. The mixture was then acidified to pH 5 with 2M aq. HCl and the solvent was removed in vacuo. The mixture was resdissolved in CH₂Cl₂ (10 mL) and washed with water (10 mL), brine (10 mL), dried (Na₂SO₄) and the solvent was removed in vacuo. The crude product was purified using flash chromatography (2:1, ethyl acetate, hexanes) to yield title product 38 (0.063 g, 62%) as a green solid. MP=98-101° C. R_{F}=0.4 (3:1 ethyl acetate, hexanes), IR vₘₐₓ (neat)/cm⁻¹ s 3449, 2938, 2865, 1695, 1205; ¹H HMR (400 MHz; CDCl₃) 1.55-1.60 (4H, m, CH₂), 1.73-1.78 (4H, m, CH₂), 1.87-1.92 (4H, m, CH₂), 2.37-2.41 (4H, m, CH₂), 3.99-4.05 (4H, m, CH₂), 6.26-6.28 (2H, m, 4-H), 6.58-6.60 (2H, m, 3-H), 6.86-6.89 (2H, m, 5-H), 7.06 (2H, s, 3′-H), 9.80 (2H, s, NH); ¹³C NMR (100 MHz; CDCl₃) 24.3 (CH₂), 25.6 (CH₂), 29.0 (CH₂), 33.8 (CH₂), 69.7 (CH₂), 106.5 (C-3), 109.0 (C-4), 110.2 (C-3′), 118.5 (C-5), 127.9 (C-2′), 129.3 (C-2), 152.6 (C-1′), 178.7 (COON). HRMS (EI) found (MNa⁺) 491.2153. C₂₆H₃₂N₂NaO₆ requires 491.2153.

### 1.7 Example 7

This example describes the preparation and use of a polymer of the invention for DNA sensing.

Attachment of probe to monomer 200 μl of a 1000 μM solution of 6,6′-((2,5-di(thiophen-2-yl)-1,4-phenylenebis(oxy))dihexanoic acid 22 in DMSO, 1685 μl of N-hydroxysuccinimide (NHS)-1-ethyl-3-(3-dimethylaminopropyl)carbodiimide (EDC) (in PBS pH 7.4) and 15 μl poly(4-styrenesulfonic acid) (PSS) were mixed at 28° C. for 30 minutes. Then 100 μl of a 1000 μM solution of probe DNA sequence (see Table 2) was added to the solution and mixed at the same temperature for another 90 minutes to conjugate the probe to the monomer.

In a 2 ml solution the final concentration of compounds were: 50 μM probe DNA, 100 μM monomer, 200 μM NHS-EDC each and 0.0075% PSS (v/v PSS:PBS/DMSO) with a final pH of 6.0.

TABLE 2
DNA sequences (FGFR3) for the probe and the target
used in the DNA sensing experiments. The probe
and target oligonucleotide sequences used in this
example were single stranded oligonucleotides
(ssONs).

| | |
|---|---|
| Probe DNA | NH₂-(CH₂)₆-CAGTAGACGGGGGTGTCACGCGAC |
| sequence | (SEQ ID NO: 2) |
| Target DNA | GTCATCTGCCCCCACAGAGCGCTG (SEQ ID |
| sequence | NO: 8) |

Electopolymerisation

The monomer with the probe DNA attached, was then polymerised on Pt electrodes without further purification.

Electrodeposition was performed using a pulse growth technique applying 0.8 V potential for 2 pulses (25 ms per pulse). Upon polymerisation, the electrode was washed with Milli-Q water to remove excess unreacted monomer and DNA. Electrodeposition of the conducting polymer-DNA complex was confirmed by electrochemical impedance spectroscopy (EIS) measurements in the presence of 5 mM K₃[Fe(CN)₆] and K₄[Fe(CN)₆].3H₂O.

Detection of Target

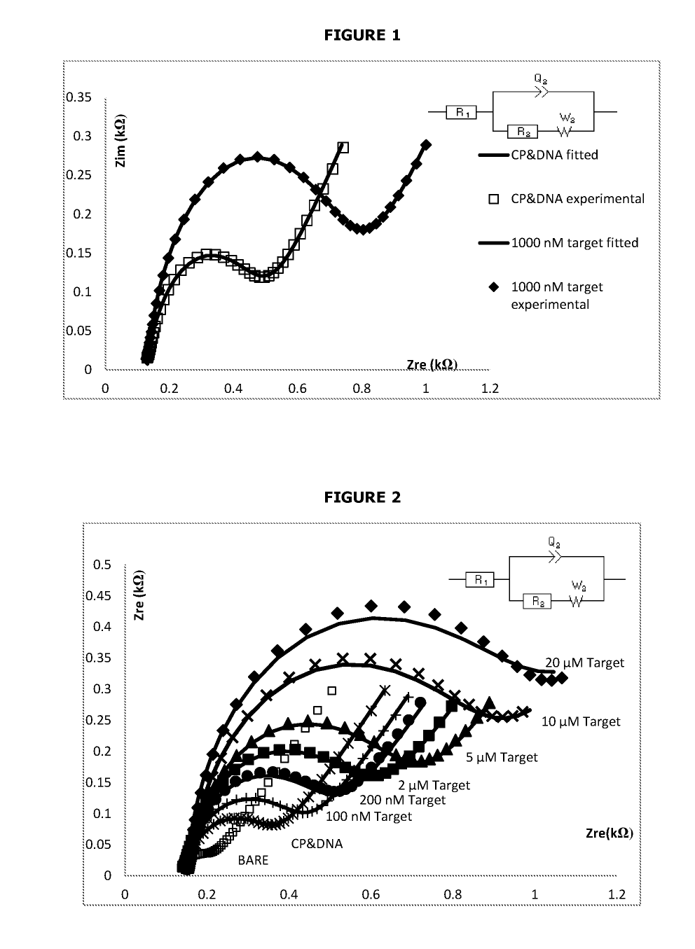

For the detection of target DNA, 1000 nM of the target DNA sequence was dropped on the electrode and the temperature was kept at 42° C. for 60 minutes. The electrode was then washed with Milli-Q water and another EIS measurement was carried out. As can be seen from FIG. 1, target hybridization led to 79% change of the charge transfer resistance of the polymer, which confirms the effective detection of target sequence.

The sensitivity of the fabricated sensor was investigated by measuring the sensor EIS response following the addition of different concentrations of the target DNA shown in Table 2 above. The results are shown in FIG. 2.

### 1.8 Example 8

24 Base sequences of oligonucleotides (ONs) of chronic lymphocytic leukemia (PBGD), bladder cancer (FGFR 3) and non-Hodgkin lymphoma (Non-Hodgkin) were purchased from Alpha DNA. Sequences are provided in Table 3. The probe and target oligonucleotide sequences used in this example were single stranded oligonucleotides (ssONs).

TABLE 3
Names and sequences of employed oligonucleotides

| Oligonucleotide | 24 base sequence from 5′ to 3′ |
|---|---|
| Non-Hodgkin probe | NH₂-(CH₂)₆-GGTCTAGCTACAGAGAAATCTCGA (SEQ |
| | ID NO: 1) |
| Non-Hodgkin target | TCGAGATTTCTCTGTAGCTAGACC (SEQ ID NO: 5) |
| Non-Hodgkin mismatch A | TCGAGATTTCTCAGTAGCTAGACC (SEQ ID NO: 6) |
| Non-Hodgkin mismatch B | TCGAGATTTCTCTCTAGCTAGACC (SEQ ID NO: 7) |
| FGFR 3 probe | NH₂-(CH₂)₆-CAGTAGACGGGGGTGTCACGCGAC |
| | (SEQ: ID NO 2) |
| PBGD probe | NH₂-(CH₂)₆-CATCTTTGGGCTGTTTTCTTCCGC (SEQ |
| | ID NO: 3) |
| PBGD target | GCGGAAGAAAACAGCCCAAAGATG (SEQ ID NO: 9) |

Monomer—DNA Coupling Procedure

All the solvents were degassed by flushing N₂ into the reaction vessel for 10 minutes prior to use. 100 μl of the monomer (either monomer 22 or 38) stock solution (200 μM in tetrahydrofuran (THF)) was pipetted into a plastic 1.5 ml eppendorf tube. 100 μl of PBS (pH 6.5) containing EDC (20 mM) and NHS (10 mM) was added to the eppendorf tube. The solution was gently shaken under N₂ in the dark for 1 hour at 28° C. An additional 100 μl of THF and 100 μl of 1 mM oligonucleotide probe solution in PBS (pH: 7.5) were then added to the eppendorf tube and the solution was mixed for another 2 hours at 28° C., under N₂, in the dark. The final solution contained 250 μM of oligonucleotide (ON), 50 μM of the monomer, 5 mM of EDC and 2.5 mM NHS in total of 400 μl of THF/PBS (pH: 7) 1:1 solution. For Fourier transform infrared spectroscopy (FTIR) and sensitivity experiments, monomer 38 was grafted with the 24 base sequence Non-Hodgkin probe to give monomer 50 and monomer 22 was grafted with the 24 base sequence PBGD probe to give monomer 60. For sensing selectivity measurements both monomers (monomer 38 and monomer 22) were grafted with Non-Hodgkin target sequences. THF was then removed under vacuum or by flushing with N₂ and the remaining aqueous solution was centrifuged at 12500 rpm for 10 minutes. The supernatant solution (containing unreacted NHS, EDC and unbound oligonucleotides) was removed by carefully pipetting it out and the solid residue was washed with excess PBS (pH 7.4) and centrifuged again for another 10 minutes. The supernatant was once more carefully removed by pipetting it out and the precipitate was dried under vacuum. The sample was kept in the freezer at −18° C. unless used immediately. The sample was used within 2 days of preparation.

In the case of coupling probes to monomer 38, weighing the monomer, stock solution preparation, oligonuceotide and THF addition were performed in a box under an atmosphere of N_{2.}

FTIR Characterisation

To confirm the covalent coupling of the oligonucleotide probes to the monomers, FTIR spectra of the monomers prior to and post attachment were recorded. The FTIR spectra were collected between 400-4000 cm⁻¹ using a Bruker Vertex 70 spectrometer in absorbance mode. To take the monomer spectrum, 100 μl of a stock solution of monomers 38 and 22 (200 μM in tetrahydrofuran (THF)) were pipetted into a plastic 1.5 ml eppendorf tube. The solution was carefully pipetted onto the sample compartment of a diamond attenuated total reflection (ATR) cell, then allowed to sit until the THF completely evaporated and the monomers precipitated onto the diamond. FTIR spectra of the monomers post-oligonucleotide probe attachment were collected by dissolving the dried samples in 100 μl of THF, pipetting the solution onto the sample compartment of the FTIR diamond, waiting for the THF to evaporate, and then recording the spectrum.

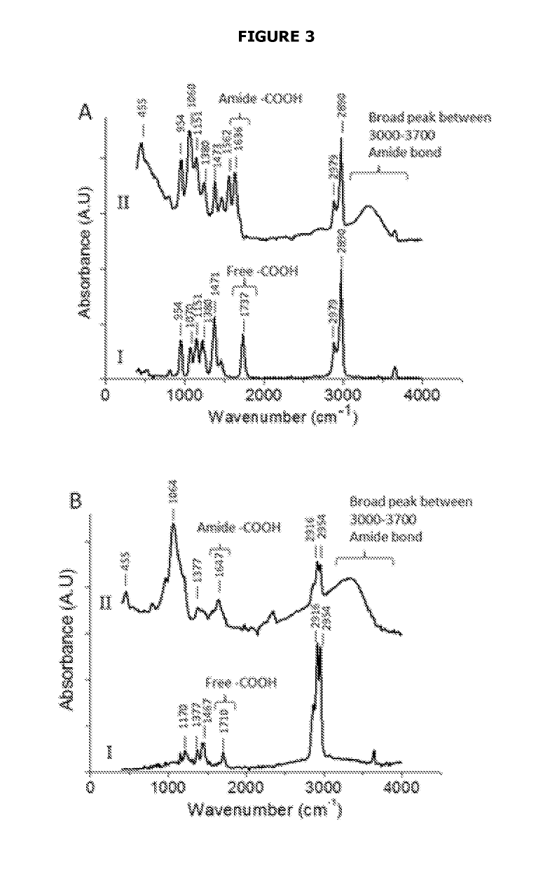

Oligonucleotide coupling of monomer 38 with the Non-Hodgkin probe and monomer 22 with the PBGD probe was confirmed by FTIR. Overlapped FTIR spectra before and after oligonucleotide probe coupling for both monomers 38 and 22 are presented in FIG. 3. Peaks at 1710 cm⁻¹ and 1737 cm⁻¹ represent the C═O stretch of carboxylic acid groups for monomer 22 and monomer 38, respectively. After the attachment these peaks were clearly shifted to 1647 cm⁻¹ and 1636 cm⁻¹ respectively, corresponding to the amide C═O stretch of DNA which the inventors believe to indicate oligonucleotide probe coupling to the carboxylic acid functionalised monomers. The broad peak between 3000 cm⁻¹ and 3700 cm⁻¹, arising from the N—H stretch, was also suggestive of amide bond formation. In the spectra of both monomer 22 and 38 there is no peak in the region up to 1000 cm⁻¹, whereas in the spectra of copolymers P70 and P80 there is a peak at 455 cm⁻¹ which may be attributed to the PO₃ stretching in DNA.

Electrochemical Deposition of the Sensing Films

The monomers with the attached oligonucleotide probes were dissolved in 200 μl of dimethyl formamide (DMF)/PBS (pH=7.4) (1:1, vol/vol). 500 μl of the 2 mM TGThP 7 solution was added into a glass vial. That solution was combined with the monomer 60 solution. 400 μl of DMF and 900 μl of PBS (pH=7.4) were added to make 2 ml of the final solution. Monomer 50 was dissolved in 200 μl of 1:1 dimethyl formamide (DMF)/PBS (pH=7.4) (1:1, vol/vol) and separately 10 mM solution of pyrrole was prepared in DMF. 100 μl of pyrrole solution was combined with the 200 μl monomer 50 solution. 1700 μl of PBS (pH=7.4) was added to make 2 ml of the final solution. In both cases, 0.0388 g of sodium para-toluene sulfonate (NaToS) was weighed into the reaction solutions and mixed until the salt was fully dissolved. Thus, the final solution contained 10 μM of monomer 60 (or monomer 50), 500 μM of TGThP 7 (or pyrrole), and 0.1 M sodium para-toluene sulfonate in 2 ml of DMF/PBS (1:1) mixture. Based on the amount of starting monomers used, the calculated ratio of the oligonucleotide-grafted monomers to either TGThP 7 or pyrrole was 1:50 mol/mol. The monomers were co-polymerised onto a 2 mm diameter gold disk electrode (BASI) in the case of monomer 60 and onto a 3 mm diameter GC electrode in the case of monomer 50, in a 3-electrode electrochemical cell containing a leak-free Ag/AgCl (3 M KCl) reference electrode (+0.242 V vs. standard hydrogen electrode SHE) and Pt wire counter electrode, by applying 0.8 V until 3.0 (±0.5) pC charge was passed, which took approximately 0.5 s.

For electrochemical characterization, cyclic voltammetry (CV) of the copolymer of monomer 50 with pyrrole (copolymer P70) and a copolymer of monomer 60 with monomer 7 (copolymer P80) were recorded in a monomer free solution of PBS at different scan rates (100 to 500 mV/s).

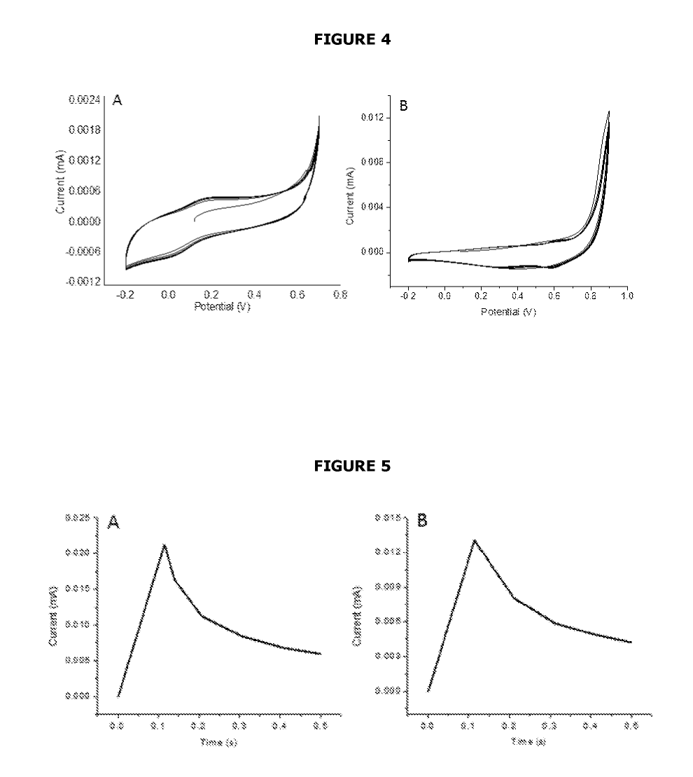

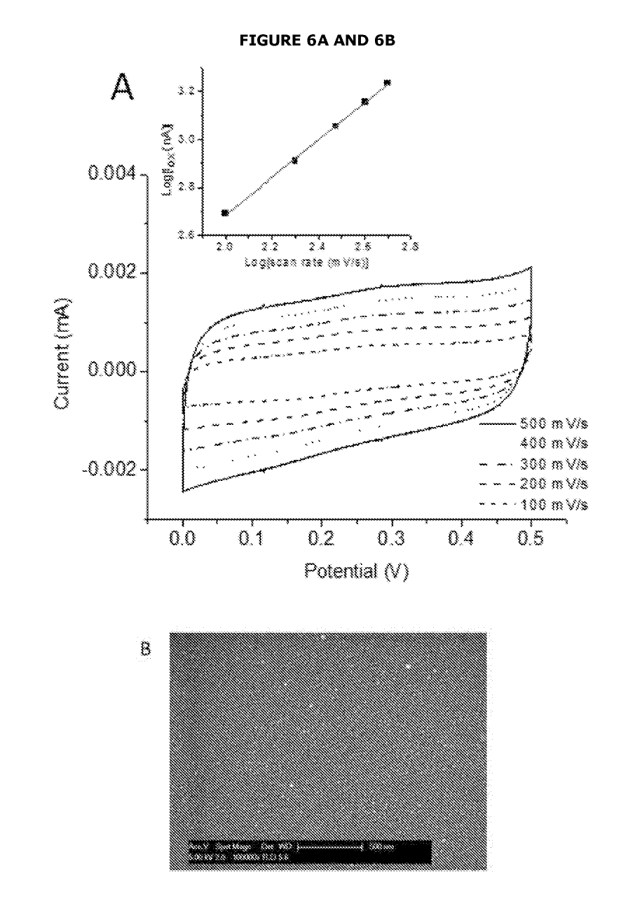

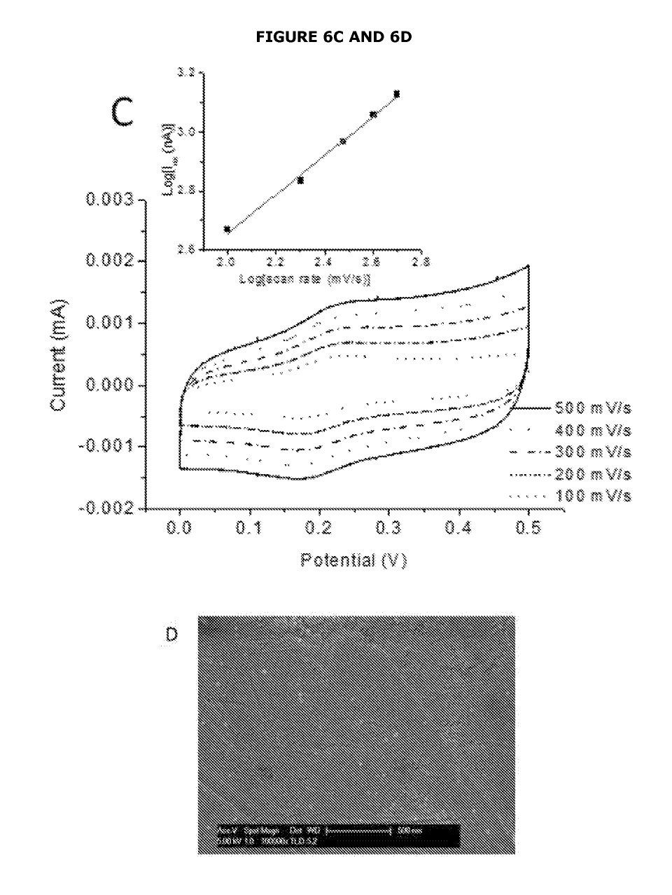

After oligonucleotide coupling to the carboxylic acid functionalised monomers, electrocopolymerisation CVs of the copolymer P80—(TGThP 7: ThPhON 60 50:1 mol/mol) on a gold electrode and of P70 (Py: PyPhON 50:1 mol/mol) on a GC elect—rode—showed copolymer formation with onset at 0.8 V and 0.6 V, respectively (FIGS. 4A and 4B). Based on these results, the sensor films for the further study were electrocopolymerised at a constant potential of 0.8 V. Oligonucleotide-functionalised copolymer films formed in 0.5 s (FIG. 5). They showed electroactivity in PBS buffer (pH 7.4), as seen in FIGS. 6A and 6C. Dependence of oxidation currents for these oligonucleotide-functionalised films versus scan rate, in a log (Iₒₓ)-log (scan rate) graph, further confirmed the electrochemical activity of the polymerised films, can be seen in the insets of FIGS. 6A and 6C. Electron microscopy images (FIGS. 6B and 6D) show the porous morphology of the films, which the inventors believe is important for achieving a high detection sensitivity for DNA hybridisation.

DNA Sensing Experiments

The fabricated sensing electrodes comprising copolymers P70 and P80 functionalised with oligonucleotides were tested by incubating the electrodes in solutions of increasing concentration of the complementary target sequences (5′ GCGGAAGAAAACAGCCCAAAGATG 3′ (SEQ ID NO: 9), the PBGD target oligonucleotide sequence) for copolymer P80 and (5′TCGAGATTTCTCTGTAGCTAGACC 3′ (SEQ ID NO: 5), the Non-Hodgkin target oligonucleotide sequence) for copolymer P70. The incubation (hybridization) was performed at 42° C. for 60 minutes for each target concentration. The electrodes were washed with deionised water (Milli Q, 18.2 MOhm.cm), followed by PBS (pH 7.4) and electrochemical impedance spectroscopy (EIS) measurements were carried out in the presence of K₃[Fe(CN)₆] and K₄[Fe(CN)₆] (5 mM each) redox couple. The EIS curves were fitted with a Randle's equivalent circuit, as shown in the inset of FIGS. 8B and 8D, where Rₛ represents solution resistance, CPE a constant phase element, R_{CT} the charge transfer resistance and W the Warburg diffusion element. The obtained values of R_{CT} were normalised to the R_{CT0} (R_{CT} for the film before incubation with the target oligonucleotide-containing solutions), and plotted in dependence on log[c(oligonucleotide)].

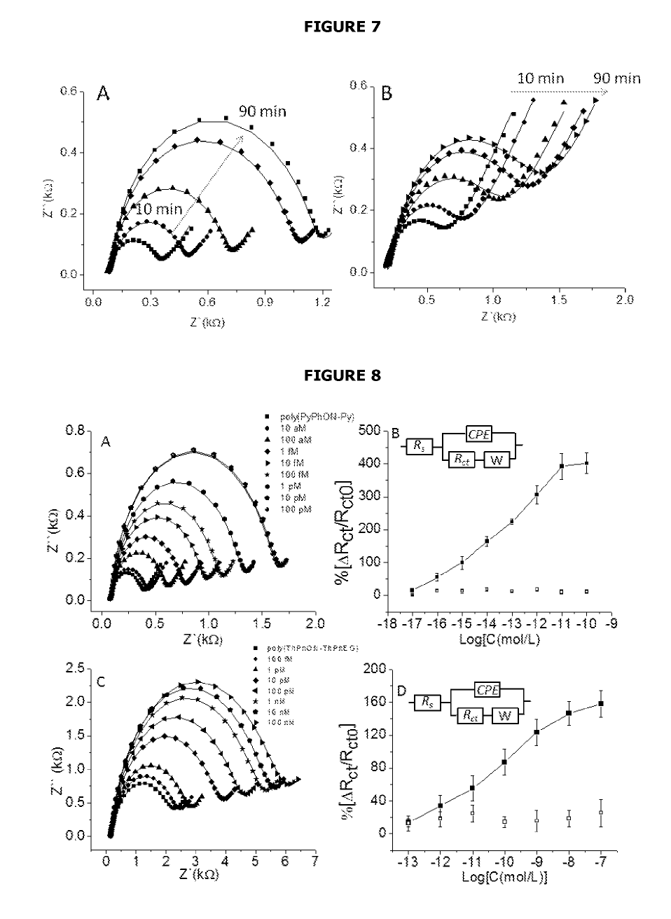

Detection of the oligonucleotide targets, present in the buffer solutions, was investigated by electrochemically probing the sensor films by electrochemical impedance spectroscopy (EIS). EIS was carried out in PBS (pH: 7.4) containing [Fe(CN)₆]^{3-/4-} (5 mM each) after 60 min of incubation of the films in target-containing buffers. 60 min was chosen as a study on the hybridisation kinetics (FIG. 7) showed that by that time >70% of the maximum EIS signal was obtained. A range of concentrations of the complementary target solutions of PBGD and Non-Hodgkin ssON target sequences were introduced onto the copolymer P80 and copolymer P70 modified electrodes, respectively. FIGS. 8A and 8C show impedance diagrams for copolymer P70 and copolymer P80 electrodes respectively, in PBS (pH: 7.4) containing [Fe(CN)₆]^{3-/4-} (5 mM each), incubated with increasing concentration of the complementary target solutions. It is notable that fM range concentrations of the Non-Hodgkin sequence (shown in FIG. 8A) resulted in an observable change in impedance of the copolymer P70 modified electrode and that pM concentrations of PBGD sequence resulted in a distinguishable change in EIS spectra of the copolymer P80 modified electrode (as shown in FIG. 8C). Normalised changes in the values of the fitted charge transfer resistances ΔR_{CT}/R_{CT}⁰, taken as the sensor response, are presented in FIGS. 8B and 8D. The inventors believe that the increase in ΔR_{CT}/R_{CT}⁰ may be attributed to specific oligonucleotide probe—target Watson-Crick base pairing. To examine the extent of any variation in the EIS properties of the films themselves in the absence of DNA hybridisation during the time required for hybridisation, the films were incubated in only PBS (pH: 7.4) without any target sequences present in the solution. Negligible change was observed for both of the polymer films (hollow symbols in the graphs of FIGS. 8B and 8D which represent these blank experiments for each of the different target concentrations).

Detection of very low concentrations of complementary sequences was achieved with these sensor films, along with dynamic range before signal saturation of 5-6 orders of magnitude. Calculations to determine the minimum detectable concentration indicated that 1.6×10⁻¹⁷ M (16 aM) of Non Hodgkin target could be detected with copolymer P70 and 4×10⁻¹³ M (0.4 pM) of PBGD target could be detected with copolymer P80. From the linear range of the sensor response (FIGS. 8B and 8D), sensitivity of 0.341 units/[log (conc./M)] was calculated for P80 sensor and 0.841 units/[log (conc./M)] for the P70 sensor. The inventors believe that the almost 4 orders of magnitude lower detection limit and 2.5 times higher sensitivity of the P70 sensing film compared to that of P80 may be due to the higher hydrophilicity, and therefore better electroactivity and a more nanoporous morphology of P70 sensing film.

### 1.9 EXAMPLE 9

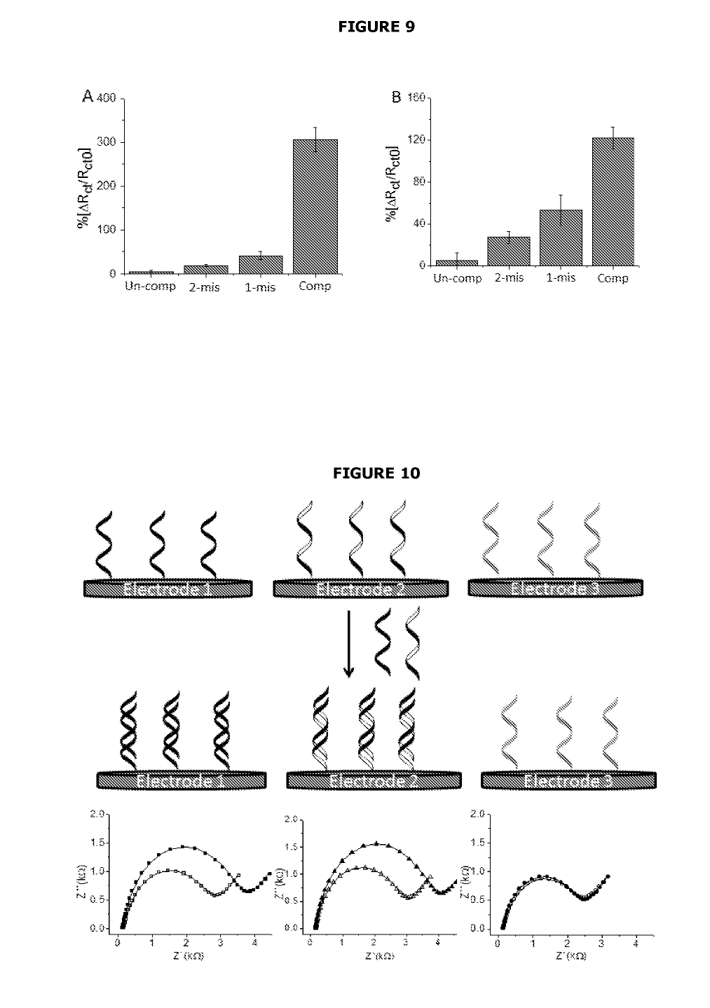

Selectivity of P91 (poly(PyPhON-co-Py), polymer formed by the attachment of the Non-Hodgkin probe (see Table 3) to monomer 38 and co-polymerising with pyrrole) and P92 (poly(ThPhON-coThPhEG), polymer formed by the attachment of the Non-Hodgkin probe (see Table 3) to monomer 22 and copolymerising with monomer 7) modified electrode sensor films towards the fully complementary target oligonucleotides was investigated by incubating the films in solutions of the fully complementary sequence (Non-Hodgkin gene), a first base mismatched Non-Hodgkin sequence (Non-Hodgkin mismatch A, see Table 3), a second base mismatched Non-Hodgkin sequence (Non-Hodgkin mismatch B, see Table 3) and fully non-complementary oligonucleotide sequences. The fully non-complementary target was a 24 base sequence of the PBGD target (see Table 3). For selectivity studies of the P91 sensing films, 1 pM concentrations of the oligonucleotides were used, while for P92 sensing films, 1 nM solutions of each oligonucleotide were used. The different concentrations were chosen from the high concentration region of the linear ranges of the sensor responses, which were different for each sensor as shown in FIGS. 7A and 7B). For P91 sensing films the response signals as a fraction of that for the fully complementary oligonucleotide were 1.4%, 6% and 13% for non-complementary, Non-Hodgkin mismatch B and Non-Hodgkin mismatch A ONs (see Table 3) respectively (FIG. 9A). These results demonstrate the selectivity of the sensor film. In the case of P92 sensing film, 1 nM of the non-complementary, Non-Hodgkin mismatch B and Non-Hodgkin mismatch A oligonucleotide (see Table 3) solutions led to response signals of 4.4%, 22.1% and 43.4% respectively, as a fraction of the signal observed in response to the fully complementary Non-Hodgkin sequence (FIG. 9B).

### 1.10 Example 10

This example shows a process for fabrication of sensing films that involves simultaneous sensing film deposition and probe immobilisation on the electrode surface. This procedure can be adapted for fabrication of gene array sensors comprising a ‘library’ of oligonucleotides pre-attached to monomers that can be immobilised onto electrode surfaces. Monomers carrying three different oligonucleotide probes—sequences from Non-Hodgkin (monomer 61), PBGD (monomer 60) and FGFR3 (monomer 62) were synthesised by reacting monomer 22 with each of the probe sequences shown in Table 3. Monomers 60, 61, and 62 were then copolymerised with monomer 7 to give the resulting copolymer thin films P63, P64 and P65 respectively, each of which were deposited on different gold electrodes. These sensing films were incubated with a PBS solution containing two target oligonucleotides at concentrations of 1 pM for each of Non-Hodgkin and PBGD genes (FIG. 10). Subsequent EIS measurements indicated strong signals from the two sensing films bearing probes for Non-Hodgkin and PBGD genes, with a sensor response of 37% and 35% respectively. A small change was observed in the EIS spectrum of the sensing film carrying the sequence probe complementary to FGFR3 (ca. 1%).

### 1.11 Example 11

This example shows the polymerisation of monomers on a carbon surface using electroless oxidative polymerisation in the presence of a Pt nanoparticle catalyst.

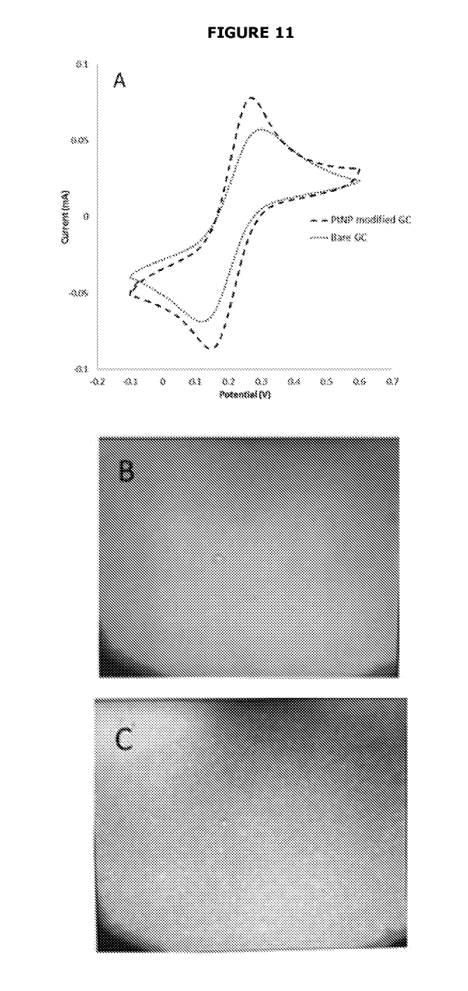

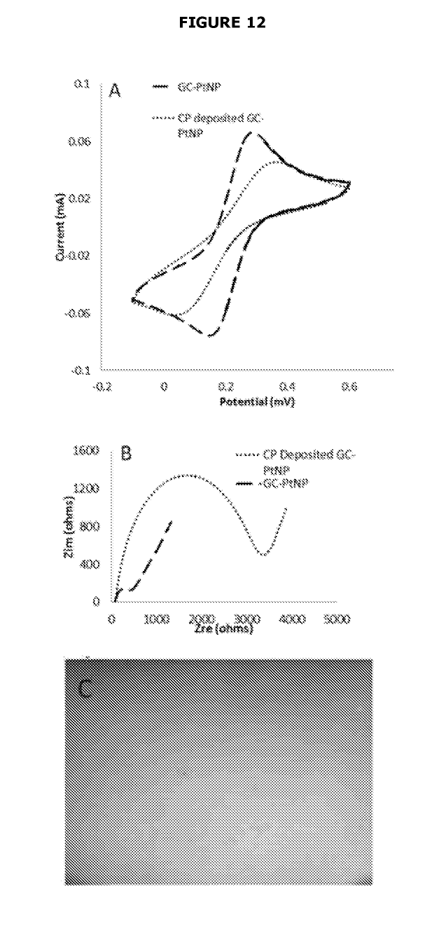

To activate the surface of the electrode, Pt nanoparticles were electrodeposited (−0.1 V for 20 seconds) on 3mm glassy carbon (GC) electrodes from a 0.5M H₂SO₄ containing 5 mM K₂PtCl₄. FIG. 11A shows the CV trace of the GC electrodes before and after Pt nanoparticle deposition. FIGS. 11B and 11C show optical pictures of the electrodes before and after deposition respectively.

In a first experiment a monomer solution containing monomer 22 and monomer 7 (10 μM:500 μM) in PBS-THF (1:1) was prepared. 10 μL of the monomer solution was drop casted on the Pt-nanparticle activated GC electrode with the Pt nanoparticles on its surface and washed after 3 minutes. The change in CV and EIS confirms the formation of the copolymer on the electrode surface.

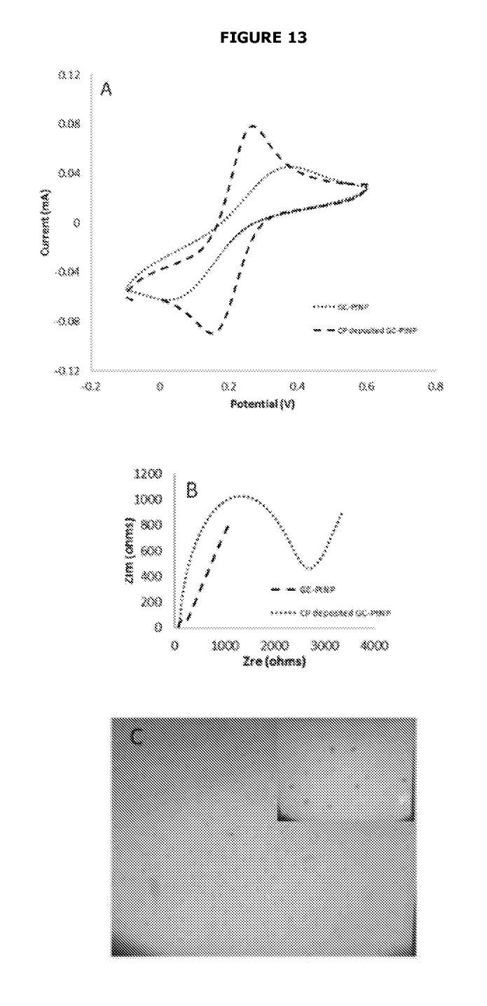

In a second experiment, a monomer solution containing monomer 22 and monomer 7 (10 μM:500 μM) in PBS-THF (1:1) and 0.1M sodium tosylate (NaToS) which acts as the dopant for the conducting polymer, was prepared. 10 μL of the monomer solution was drop casted onto the Pt nanoparticle-activated carbon electrode and washed after 3 minutes. The CV traces and EIS spectra for the ferro-ferricyanide redox reaction and optical pictures of the electrodes resulting from this experiment are shown in FIGS. 13A-C respectively. The flattening of the CV trace and the increase of electrode impedance both confirm coating of the electrode by the copolymer film.

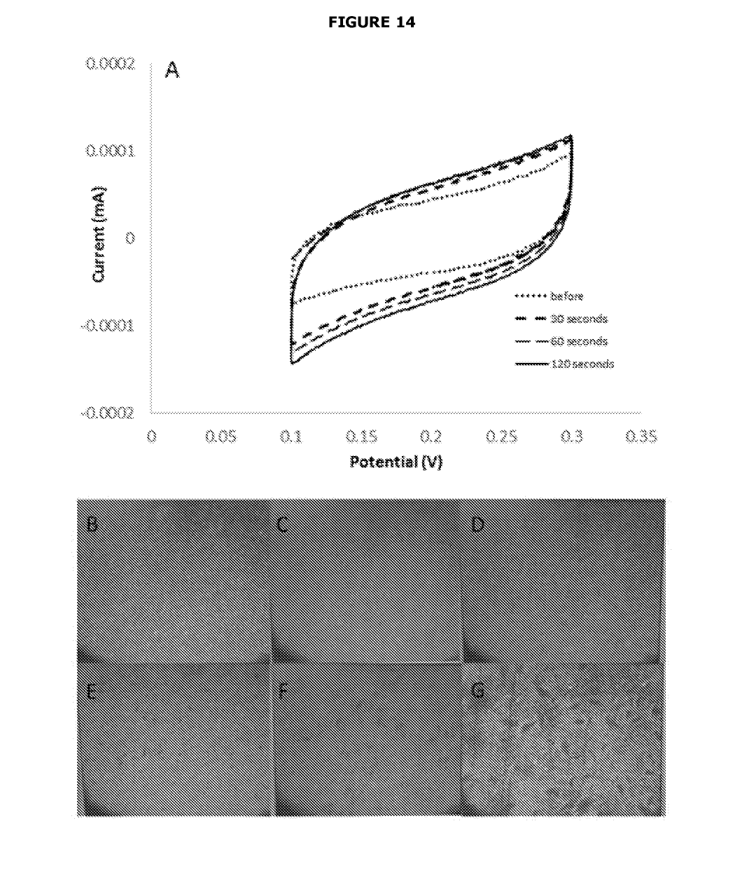

In a third experiment, to investigate polymerisation kinetics a monomer solution containing oligonucleotide-functionalised monomer 60 and monomer 7 (10 μM:500 μM) in PBS-THF (1:1) and 0.1M NaToS was prepared. 10 μL of the monomer solution was drop casted onto the Pt nanoparticle-activated carbon electrode and washed after 30, 60, 120, 240, 360 seconds. The CV traces obtained for the ferro-ferricyanide redox reaction are shown in FIG. 14A. FIGS. 14B-G show pictures of the electrodes obtained before (14B) and after 30 (14C), 60 (14D), 120 (14E), 240 (14F), 360 (14G) seconds of polymerisation respectively. Measurement of the EIS spectrum for the ferro-ferricyanide reaction gave a value for the charge transfer resistance for this reaction on the polymer-modified electrode.

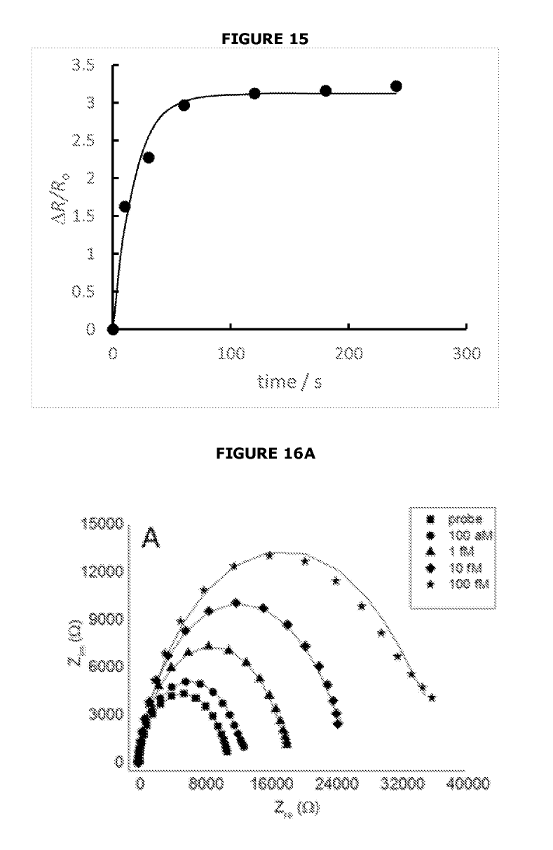

As shown by the relative change in charge transfer resistance in FIG. 15, polymerisation induced by air on a Pt nanoparticle-activated glassy carbon surface of a mixture of monomers 60 and 7 may be achieved within 60 seconds.

### 1.12 Example 12

This example shows the polymerisation of monomers on a screen-printed carbon electrode using electroless oxidisation in the presence of a Pt nanoparticle catalyst, and the use of the modified electrode to detect hybridization of a complementary sequence. The DRP150 screen printed electrode assemblies from DropSense that were used for this proof-of-principle demonstration comprise a central carbon disc working electrode, a surrounding arc of Ag/AgCl normally used as a reference electrode, and a further surrounding arc of carbon as the counter electrode. These were operated as a two-terminal system, utilizing only the two carbon electrodes.

A dispersion of platinum nanoparticles in ethanol was prepared as follows. 2.4 mg of K₂PtCl₆ was dissolved in 5 mL of distilled water (5 μmol) and stirred under nitrogen for 15 min at 1000 rpm. Then, 20 μL of a 2 M NaBH₄ solution in triethylene glycol dimethyl ether (40 μmol) was added in one shot. A dark precipitate appeared immediately. The mixture was stirred at 1000 rpm for a further 15 min under N₂ atmosphere. The reaction mixture was sonicated, centrifuged, and the supernatant solution was removed. The Pt nanoparticles (PtNPs) were then washed with ethanol and centrifuged. They were then suspended in ethanol and sonicated for 10 minutes prior to use. For the activation of printed carbon electrodes, 5 μl of PtNP dispersion in ethanol was dropped onto each working electrode. The solvent completely evaporated within 30 seconds.

5 μL of solution containing 10 μM monomer 60 (ThPhON with PBGD probe), 500 μM monomer 7 and 0.1 M NaTos in (1:1) THF:PBS (pH: 7.4) was drop cast onto the PtNP modified printed carbon working electrode and washed after 60 seconds. An increase in charge transfer resistance measured in a mixture of 5 mM each of K₄Fe(CN)₆ and K₃Fe(CN)₆ in PBS (2-terminal measurement with potential difference between the two carbon electrodes=0V) confirmed the formation of copolymer P90 on the electrode surface. Electrodes were then incubated with a 1×10⁻¹² M solution of the complementary sequence, in PBS containing ferro-ferricyanide at 42° C. for 1hr. The charge transfer resistance increased, signally the hybridisation of the complementary sequence onto the surface-attached probe sequence: ΔR_{ct}/R⁰_{ct}=67%. Incubation with non-complementary sequence (1pM) gave ΔR_{ct}/R⁰_{ct}=7%.

It is not the intention to limit the scope of the invention to the abovementioned examples only. As would be appreciated by a skilled person in the art, many variations are possible without departing from the scope of the invention.

In addition, where features or aspects of the invention are described in terms of a Markush group, those skilled in the art will recognise that the invention is also thereby described in terms of any individual member or subgroup of members of the Markush group.

### 1.13 Example 13

This example demonstrates the use of two copolymers, P100 (P(PyPhON-Py) and P200 (P(PyPhON-PyPhEG)), comprising a ssON F1630 probe in sensors for the detection of (i) synthetic E. coli DNA and (ii) extracted genomic E. coli BL21 DNA. The ssON probe was 5′ NH₂—(CH₂)₆-CTAGTTTAGACAGCTAGGAAGG 3′ (SEQ ID NO: 4).

The E. coli BL21 genome (GenBank CP001509.3) contains a sequence (CTAGTTTAGACA (SEQ ID NO: 11)) that is 100% identical to the 5′ region of the ssON F1630 probe.

The ssON F1630 probe and target DNA sequences and the Non-Hodgkin ssDNA probe sequences used in this and subsequent examples were sourced from Alpha DNA.

General Procedures

Monomer DNA Attachment

Monomer DNA attachment was carried out as follows.All the solvents were degassed by flushing with N₂ into the reaction vessel for 10 minutes prior to use. 100 μL of the monomer 38 stock solution (200 μM in tetrahydrofuran (THF)) was pipetted into a plastic 1.5 mL eppendorf tube. 100 μL of PBS (pH 6.5) containing EDC (20 mM) and NHS (10 mM) was added to the eppendorf tube. The solution was gently shaken under N₂ in the dark for 1 hour at 28° C. An additional 100 μL of THF and 100 μL of 1 mM ssON F1630 probe solution (5′ NH₂—(CH₂)₆-CTAGTTTAGACAGCTAGGAAGG 3′ (SEQ ID NO: 4)) in PBS (pH: 7.5) were then added to the eppendorf tube and the solution was mixed for another 2 hours at 28° C., under N₂, in the dark. The final solution contained 250 μM of oligonucleotide (ON), 50 μM of the monomer, 5 mM of EDC and 2.5 mM NHS in total of 400 μL of THF/PBS (pH: 7) 1:1 solution. THF was then removed under vacuum or by flushing with N₂ and the remaining aqueous solution was centrifuged at 12500 rpm for 10 minutes. The supernatant solution (containing unreacted NHS, EDC and unbound oligonucleotides) was removed by carefully pipetting it out and the solid residue was washed with excess PBS (pH 7.4) and centrifuged again for another 10 minutes. The supernatant was once more carefully removed by pipetting it out and the precipitate was dried under vacuum. The sample of the product monomer 80 was kept in the freezer at −18° C. unless used immediately. The sample was used within 2 days of preparation.

Monomer 90 was prepared in the same manner as described above for monomer 80, except with single-stranded Non-Hodgkin probe sequence (5′ NH₂—(CH₂)₆-GGTCTAGCTACAGAGAAATCTCGA 3′ (SEQ ID NO: 1)) attached to monomer 38 instead of the ssON F1630 probe.

In the case of coupling probes to monomer 38, weighing the monomer, stock solution preparation, oligonuceotide and THF addition were performed in a box under an atmosphere of N₂.

Electrochemical Deposition of the Sensing Films

P100 (PyPhON-Py) and P200 (PyPhON-PyPhEG) were formed by polymerising 10 μM of monomer 80 with 500 μM pyrrole (for P100) and 10 μM of monomer 80 with 500 μM of monomer 33 (for P200) respectively, via applying 0.8 V potential for 0.5s in a three terminal electrochemical cell where glassy carbon (GC) was the working electrode, platinum (Pt) coil was the counter electrode (CE) and leakless Ag/AgCl was the reference electrode (RE). Upon polymerisation electrodes were removed from the solution immediately and washed with PBS (pH: 7.4).

P300 and P400 were formed in the same manner as described above, except that 10 μM of monomer 90 was polymerised with 500 μM pyrrole (for P300) and 10 μM of monomer 90 was polymersied with 500 μM of monomer 33 (for P400) respectively.

Treatment Before Target Incubation

After the polymerisation, three cycles of CV were performed on the films in PBS (pH=7.4) between 0-0.3V to allow conditioning of the sensing films prepared by the electrochemical deposition of the sensing films as described above. Before incubation of the sensor with the target oligonucleotide solution, the electrodes were kept in PBS at 42° C. for 1 hour.

Electrochemical Measurements

EIS measurements were performed in PBS containing K₃[Fe(CN)₆] and K₄[Fe(CN)₆] (5 mM each) redox couple in a three terminal electrochemical cell again where, depending on the experiment, P100, P200, P300 or P400 on glassy carbon electrode was the working electrode (WE), Pt coil was counter electrode (CE) and leakless Ag/AgCl was the RE.

A frequency range 100 kHz-0.1 Hz was scaned at an applied bias potential of 0.23V using leakless Ag/AgCl RE.The EIS curves were fitted with a Randle's equivalent circuit (identical to that shown in the inset of FIG. 8), where Rₛ represents solution resistance, CPE a constant phase element, R_{CT} the charge transfer resistance and W the Warburg diffusion element. The obtained values of R_{CT} were normalised to the R_{CT0} (R_{CT} for the film before incubation with the target oligonucleotide-containing solutions).

Extracted Genomic E. coli BL21 DNA Target Sample Preparation—Procedure for Growth of Cell Culture, Lysis and Genomic DNA Extraction from the Cellular Lysate

BL21 strain of Escherichia coli (E. coli) was grown in 2.5 mL lysogeny broth (LB) for 18 h. Bacteria in the LB medium were transferred to PCR tubes and lysed by heating at 95° C. for 5 min using a PCR thermo cycler. Crude bacterial lysate was prepared by diluting this heated preparation using phosphate-buffered saline (pH 7.4) to 6×10⁸ cfu/mL (equivalent to 1 pM of genomic DNA assuming 1 DNA per bacterium) then passing this mixture through a 0.22 μm filter and diluting further as required using phosphate-buffered saline (pH 7.4). Genomic E.coli DNA was obtained using commercially available DNA extraction kits (in general: bacterial lysis which may be followed by digestion with proteinase K, then extraction of DNA with phenol/chloroform/isoamyl alcohol and precipitation of DNA with sodium acetate and isopropanol).

Synthetic E. coli DNA Target Sample Preparation

The synthetic E. coli ssDNA that was used in the DNA sensing experiments in the examples herein is the F1630 sequence, 5′ CCTTCCTAGCTGTCTAAACTAG 3′ (SEQ ID NO: 10).

The synthetic E. coli DNA target samples used in the DNA sensing experiments in this example were prepared as follows. Synthetic E. coli DNA target sequence (F1630 sequence, 5′ CCTTCCTAGCTGTCTAAACTAG 3′ (SEQ ID NO: 10)) was purchased from Alpha DNA in dried/solid state and dissolved in PBS (pH: 7.4) to a concentration of 1 mM at room temperature. The stock solution obtained was further diluted to lower concentrations using PBS (pH: 7.4) as necessary.

DNA Sensing Experiments

### Synthetic E. coli DNA sensing experiments using P100 (P(PyPhON-Py)) and P200 (P(PyPhON-PyPhEG))

Electropolymerisation and treatment before target incubation of P100, P200, P300 and P400 sensing films was carried out as described above. 50 μL of the 1 mM stock solutioncomprising the synthetic E. coli DNA target prepared as described above was transferred onto electrodes comprising P100 (P(PyPhON-Py), P200 (P(PyPhON-PyPhEG), P300 or P400 and the electrodes were sealed with parafilm. Each electrode was immediately transferred to the water bath and kept at 42° C. for 1 hour. After that time, electrodes were washed with PBS and the EIS measurement was performed. After the measurement, each electrode was washed with PBS again. The electrodes were then incubated in increasing concentrations of 100 aM, 1 fM, 10 fM and 100 fM of the ssON F1630 target solution.

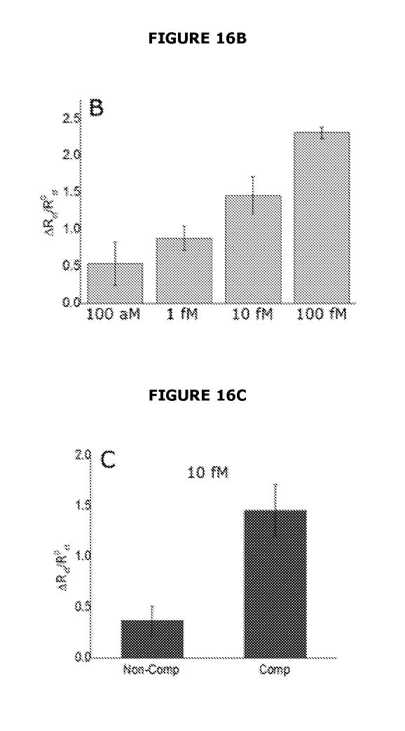

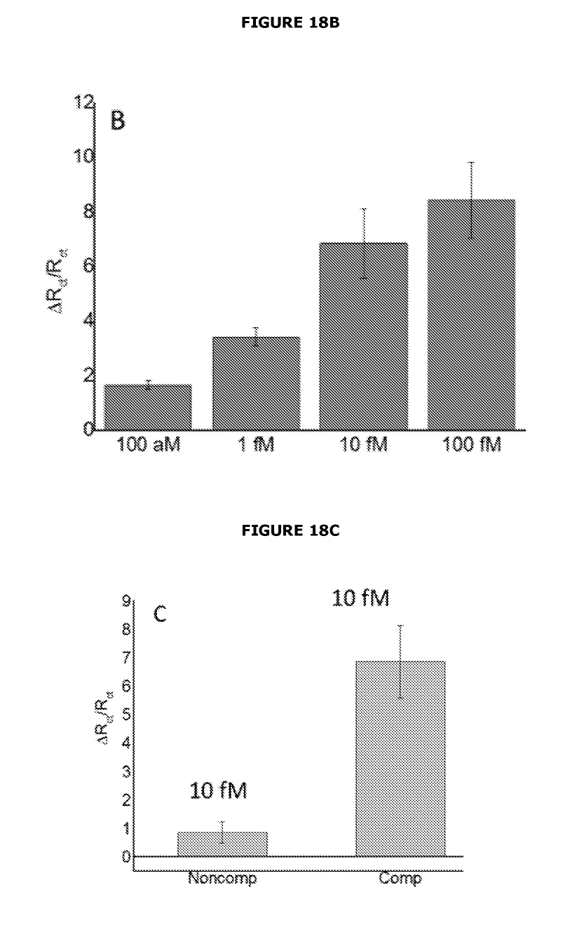

The results for these experiments are shown in FIGS. 16A-C and 18A-C. FIGS. 16A-B relate to P100, FIG. 16C relates to P100 and P300, FIGS. 18A-B relates to P200 and FIG. 18C relates to P200 and P400.

### Extracted genomic E. coli BL21 DNA Sensing Experiments using P100 (P(PyPhON-Py))

Electropolymerisation and treatment before target incubation of P100, P200, P300 and P400 sensing films was carried out as described above. 100 pL of the extracted genomic E. coli BL21 DNA target sample was kept at 95° C. for 5 min in order to denature the double-stranded DNA into single-stranded DNA. Then 50 μL of the resultant dissociated solution (100 aM) was quickly transferred onto electrodes comprising either the P100 (P(PyPhON-Py) sensing film or the P200 (P(PyPhON-PyPhEG), P300 or P400 and the electrodes were sealed with parafilm. Electrodes were immediately transferred to the water bath and incubated at 42° C. for 1 hour. The electrodes were washed with PBS and EIS measurement was performed. After the measurement, each electrode was washed with PBS again and the electrodes were then incubated in increasing concentrations of 1 fM, 10 fM and 100 fM of the extracted genomic BL21 DNA target solution prepared as described above.

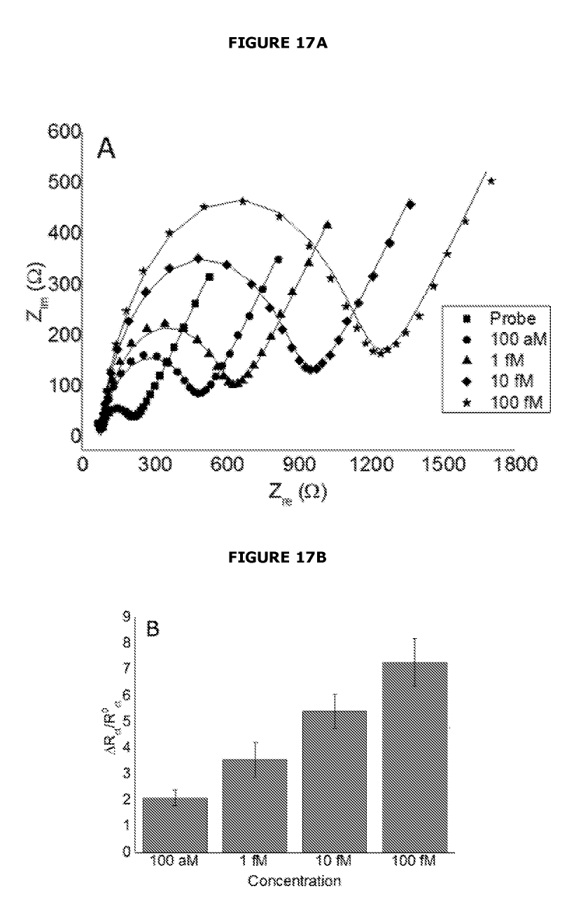

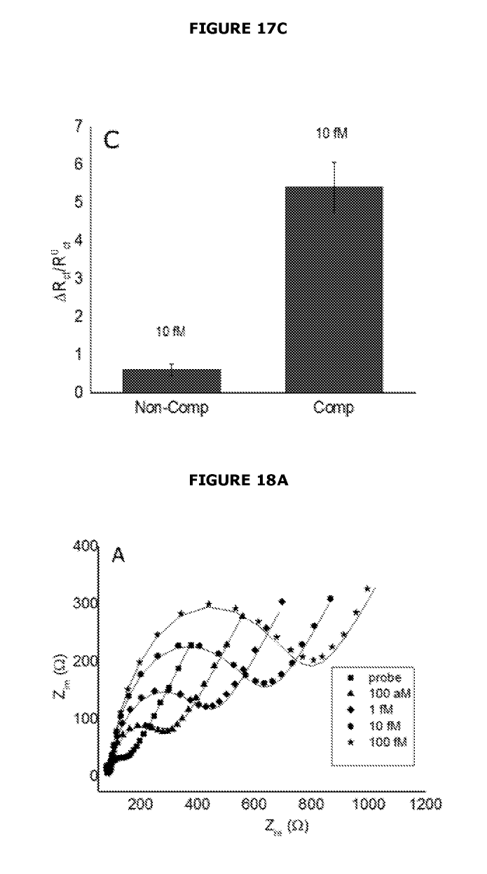

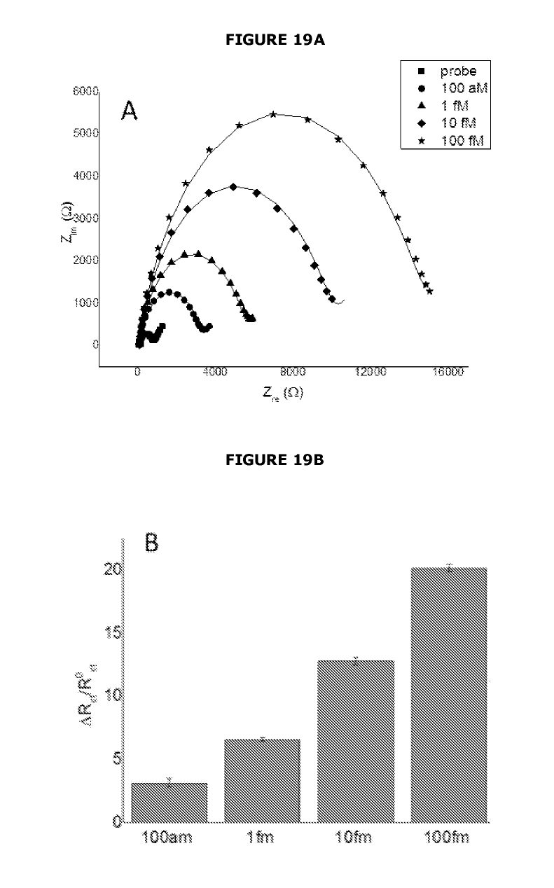

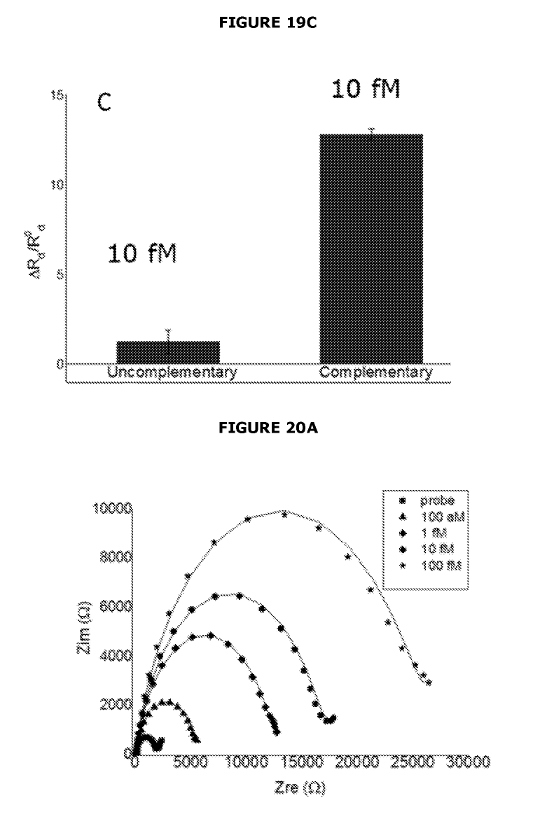

The results for these experiments are shown in FIGS. 17A-C and 19A-C. FIGS. 17A-B relate to P100, FIG. 17C relates to P100 and P300, FIGS. 19A-B relates to P200 and FIG. 19C relates to P200 and P400.

### 1.14 Example 14

Crude E. coli BL21 DNA Lysate DNA Experiments using P200 (P(PyPhON-PyPhEG)) and P400

Sensing electrodes were prepared by electropolymerisation of PyPhON (monomer 80) and PyPHEG (monomer 33) (10 μM: 500 μM) to give P200 (PyPHON-PhEG), and electropolymerisation of monomer 90 with monomer 33 (10 μM: 500 μM) to give P400.

Polymerisation was carried out onto a glassy carbon electrode by applying 0.8 V potential for 0.5s in a 3-terminal electrochemical cell where the glassy carbon electrode was the working electrode, platinum (Pt) coil was counter electrode (CE) and Ag/AgCl (3M NaCl) was reference electrode (RE). The electropolymerisation solution contained either monomer 80 and monomer 33 (10 μM: 500 μM) and 0.1M NaTos in DMF: PBS (1:1) or monomer 90 and monomer 33 (10 μM: 500 μM) and 0.1M NaTos in DMF: PBS (1:1). Upon polymerisation the electrodes were removed from the solution immediately and washed with PBS (pH: 7.4). 3 cycles of CV performed in PBS (pH: 7.4) between 0-0.3V for the stabilization of the conducting polymer-immobilised-electrodes.

Cultured E. coli BL21 bacteria were killed and lysed by heating to 95° C. for 5 min. The lysate was diluted to 1 μM and was and filtered through 220 nm filter. The filtered lysate was diluted further to provide 100 fM, 10 fM, 1 fM and 100 aM target solutions (equivalent to 6 ×10⁷, 6 ×10⁶, 6 ×10⁵, and 6 ×10⁴ CFU/mL respectively). 150 μL of the target solution was kept at 95° C. for 5 min in order to denature the DNA. Then 50 μL of target solution was immediately transferred onto each electrode and sealed with parafilm. Electrodes were immediately transferred to the water bath and kept at 42° C. for 1 hour to allow annealing of genomic DNA present in the sample to the electrode-bound probe.

Electrochemical impedance spectroscopy was performed in a phosphate-buffered saline solution containing 5 mM each of potassium ferrocyanide and potassium ferricyanide. After the hybridization, electrodes were washed with PBS and EIS measurement was performed. After the measurement, each electrode was washed with PBS again. The same procedure was applied for the consecutive target concentrations.

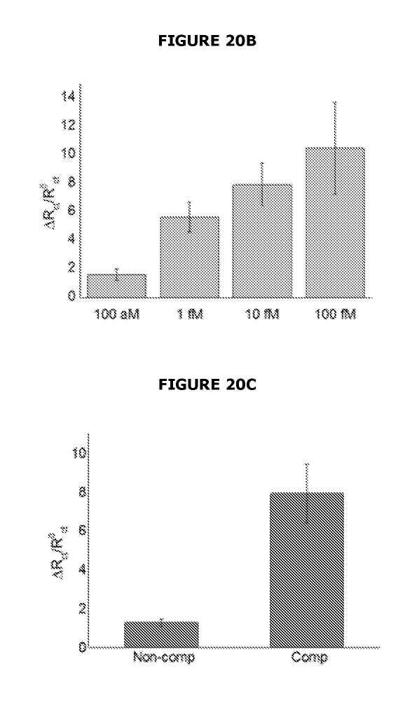

FIG. 20 shows the signal observed for the range of concentrations of tested E. coli lysate, expressed as molar concentrations. The control measurement gave a smaller, non-specific signal.

### 1.15 Example 15

This example shows the kinetics of detecting synthetic E. coli ssDNA (i.e. 5′ CCTTCCTAGCTGTCTAAACTAG 3′ (SEQ ID NO: 10)) in a sample prepared according to the synthetic E. coli target sample preparation method described above, using an electrode comprising a P200 sensing film.

Electropolymerisation and treatment before target incubation was performed as mentioned earlier.

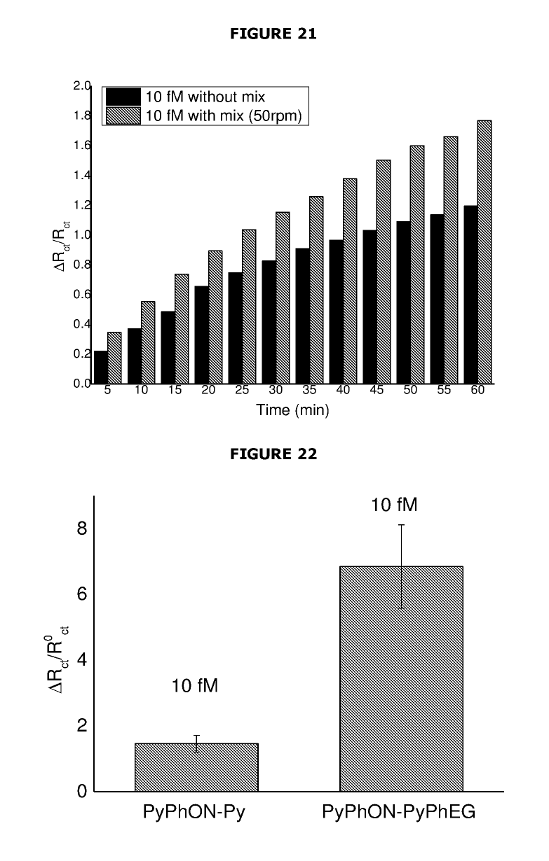

The electrode comprising the P200 sensing film was carried in a 9.9 ml PBS solution containing 5 mM [Fe (CN)₆^{3-/4-}] at 42° C. The temperature was checked with a thermometer. EIS measurement was performed in a three terminal electrochemical cell where P200 (P(PyPhON-PyPhEG) was the working electrode (WE), Pt coil was the counter electrode (CE) and leakless Ag/AgCl was the reference electrode (RE). The first measurement was performed in a target free solution. Then 100 μL of the target solution (synthetic E. coli F1630 ssDNA (5′ CCTTCCTAGCTGTCTAAACTAG 3′) (SEQ ID NO: 10) target sample as described for example 13 above) was injected in the solution to give a final concentration of 10 fM and EIS measurements were taken at 42° C. These experiments were carried out in two ways, the first being one in which the target solution was unmixed, and the second being mixing of the target solution constantly at 50 rpm, apart from when the measurements were taken. EIS measurements were performed every 5 min. FIG. 21 shows the results of these kinetics EIS measurements.

### 1.16 Example 16

This example compares the responses of sensors based on P100 (P(PyPhON-Py) and P200 (P(PyPhON-PyPhEG) to synthetic E. coli F1630 ssDNA (5′ CCTTCCTAGCTGTCTAAACTAG 3′ (SEQ ID NO: 10)) samples and extracted E. coli genomic BL21 DNA samples prepared according to the method described in example 13 respectively.

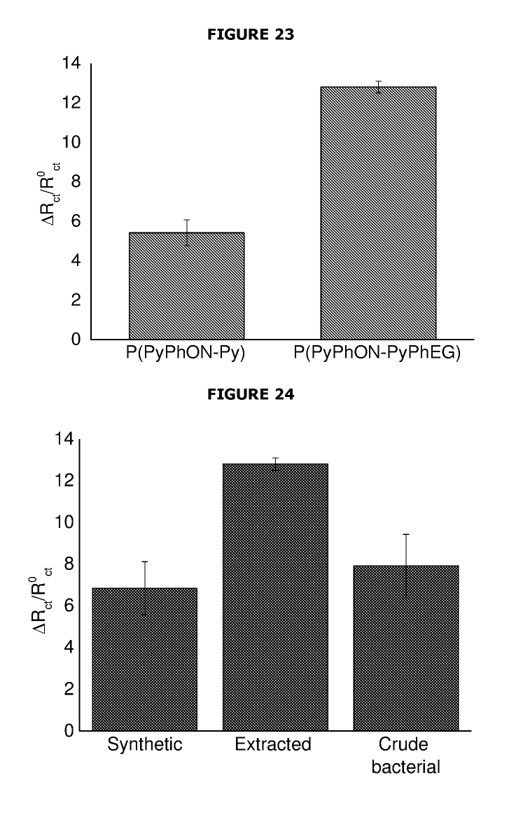

FIGS. 22 and 23 show that sensors comprising P200 showed stronger responses to both the synthetic ssON F1630 target (FIG. 22) and extracted genomic BL21 bacterial DNA target (FIG. 23) than the sensors comprising P100.

### 1.17 Example 17

This example compares the responses of sensors based on P200 (P(PyPhON-PyPhEG)) to synthetic E. coli DNA samples (synthetic), extracted E. coli genomic BL21 DNA samples (extracted) and crude BL21 E. coli lysate DNA samples (crude bacterial) prepared according to the method described in example 13 and crude BL21 E. coli DNA lysate prepared according to the method described in example 14.

FIG. 24 shows the result of this experiment. The sensor based on P200 has the strongest response to extracted E. coli genomic BL21 DNA samples.

### 1.18 Example 18

This example shows DNA sensing using a conducting polymer film made by electropolymerisation onto screen-printed carbon electrodes.

Screen-printed carbon electrodes (Gwent Electronic materials type C2100126D6—Heat Curable Carbon Paste) on a plastic substrate (Valox ® FR1) were obtained from Gwent Electronic Materials Ltd. P500 (PyPhON-PyPhEG) was polymerised by applying 0.8 V potential for 20s using a three terminal electrochemical cell where the Gwent electrode was the working electrode, platinum (Pt) coil was the counter electrode (CE) and Ag/AgCl (3M NaCl) was the reference electrode (RE). The electropolymerisation solution contained 10 μM monomer 80 (PyPhON) and 500 μM monomer 33 (PyPhEG) and 0.1M NaTos in DMF: PBS (1:1). Upon polymerisation electrodes were removed from the solution immediately and washed with PBS (pH: 7.4). 3 cycles of CV performed in PBS (pH: 7.4) between 0-0.3V for the stabilization of the conducting polymer-immobilised—Gwent electrodes. After polymerisation, detection of synthetic E. coli F1630 ssDNA target was performed by incubation with different concentrations of synthetic E. coli DNA target (5′ CCTTCCTAGCTGTCTAAACTAG 3′ (SEQ ID NO: 10)) solutions prepared as described for example 13 above at 42° C. for 1 h. EIS were measured after polymerisation and incubation with target solutions.

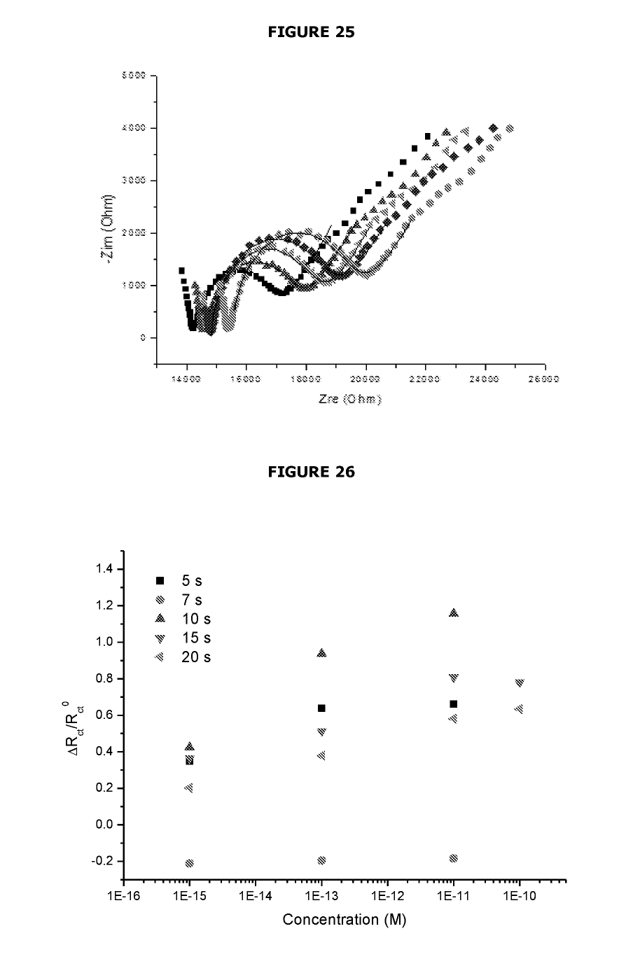

FIG. 25 shows that there is a systematic increase of impedance caused by hybridisation of the target sequence (synthetic E. coli DNA target (5′ CCTTCCTAGCTGTCTAAACTAG 3′ (SEQ ID NO: 10)) to the electrode. Sensitivity to very low concentrations of target is evident.

FIG. 26 also shows the effect of polymerisation time on DNA sensing using-P500 (p(PyPhON-PyPhEG)) modified Gwent electrodes after incubation in PBS buffer with 1 fM, 100 fM and 10 pM synthetic E. coli DNA target sample (5′ CCTTCCTAGCTGTCTAAACTAG 3′ (SEQ ID NO: 10)). The results show that the sensor response can be optimised by depositing varying amounts of the P(PyPhON-PyPhEG) (P500).

It is not the intention to limit the scope of the invention to the abovementioned examples only. As would be appreciated by a skilled person in the art, many variations are possible without departing from the scope of the invention.

### REFERENCES

(1) Brunner, H.; Gruber, N. Inorganica Chim. Acta 2004, 357, 4423-4451.
(2) Winkler, B.; Dai, L.; Mau, A. W.-H. Chem. Mater. 1999, 11 (3), 704-711.
(3) Ogawa, K.; Chemburu, S.; Lopez, G. P.; Whitten, D. G.; Schanze, K. S. Langmuir 2007, 23, 4541-4548.
(4) Ko, S.-B.; Cho, A.-N.; Kim, M.-J.; Lee, C.-R.; Park, N.-G. Dyes Pigments 2012, 94, 88-98.
(5) Lee, J.-M.; Kim, J.; Shin, Y.; Yeom, C.-E.; Lee, J. E.; Hyeon, T.; Moon Kim, B. Tetrahedron Asymmetry 2010, 21, 285-291.
(6) Vandeleene, S.; Verswyvel, M.; Verbiest, T.; Koeckelberghs, G. Macromolecules 2010, 43, 7412-7423.
(7) Sakamoto, N.; Ikeda, C.; Yamamura, M.; Nabeshima, T. Chem. Commun. 2012, 48, 4818-4820.

1. A polymerisable monomer of formula (1): 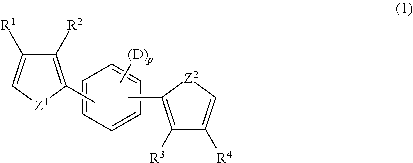
wherein
p is 1 or 2;
R¹, R², R³ and R⁴ are each independently selected from the group consisting of hydrogen, an electron withdrawing group and an electron donating group; or
R¹ and R² together and/or R³ and R⁴ together represent an electron withdrawing group or an electron donating group that together with the atoms to which they are attached form a five or six membered ring;
D at each instance of p is independently a group of the formula -L-PX, wherein L is a bond or a linker group, and Px is a probe capable of binding one or more nucleic acids or comprising a nucleic acid or an analogue thereof;
Z¹ and Z² are each independently S or NR^{a}; and
R^{a} at each instance is independently selected from the group consisting of hydrogen and alkyl.

2. The polymerisable monomer of claim 1, wherein the polymerisable monomer has the formula (1A): 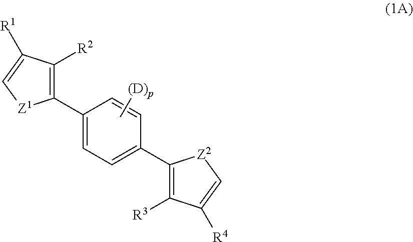
wherein p, R¹, R², R³, R⁴, D, Z¹, and Z² are as defined in claim 1.

3. The polymerisable monomer of any one if the preceding claims, wherein p is 2.

4. The polymerisable monomer of claim 3, wherein each D is identical.

5. The polymerisable monomer of any one of the preceding claims, wherein the polymerisable monomer has the structure (1B): 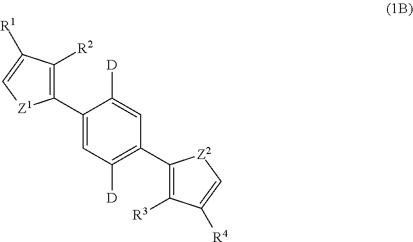
wherein R¹, R², R³, R⁴, D, Z¹, and Z² are as defined in claim 1.

6. The polymerisable monomer of any one of the preceding claims, wherein
R¹, R², R³, and R⁴are each independently selected from the group consisting of hydrogen, halo, nitro, nitrile, —C(O)R⁵, —OR⁵, —C(O)OR⁵, —OC(O)R⁵, —NR⁵R⁵, —C(O)NR⁵R⁵, —NR⁵C(O)R⁵, —NR⁵C(O)NR⁵R⁵, and —R⁶; or
R¹ and R² and/or R³ and R⁴ together with the atoms to which they are attached form a five or six membered heterocyclic or carbocyclic ring;
R⁵ at each instance is independently selected from the group consisting of hydrogen, alkyl, alkenyl, cycloalkyl, cycloalkenyl, aryl, heterocyclyl, and heteroaryl; and
R⁶ at each instance is independently selected from the group consisting of alkyl, alkenyl, cycloalkyl, cycloalkenyl, aryl, arylalkyl, heterocyclyl, and heteroaryl, each of which is optionally substituted with one or more substituents independently selected from halo, nitro, nitrile, —C(O)R⁵, —OR⁵, —C(O)OR⁵, —OC(O)R⁵, —NR⁵R⁵, —C(O)NR⁵R⁵, —NR⁵C(O)R⁵, —NR⁵C(O)NR⁵R⁵, and alkyl.

7. The polymerisable monomer of any one of the preceding claims, wherein R¹, R², R³, and R⁴ are each hydrogen; or R¹ and R² together and/or R³ and R⁴ together represent —OCH₂CH₂O—.

8. The polymerisable monomer of any one of the preceding claims, wherein R¹ and R⁴ are identical and R² and R³ are identical; or when R¹ and R² form a ring and R³ and R⁴ form a ring, each ring is identical.

9. The polymerisable monomer of any one of the preceding claims, wherein R¹, R², R³, and R⁴ are each hydrogen.

10. The polymerisable monomer of any one of the preceding claims, wherein Z¹ and Z² are each S; or Z¹ and Z² are each NR^{a}.

11. The polymerisable monomer of any one of the preceding claims, wherein R^{a} at each instance is hydrogen.

12. The polymerisable monomer of any one of the preceding claims, wherein the polymerisable monomer has the formula (1C) or (1D): 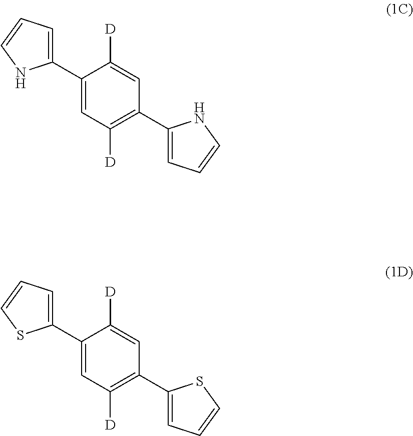
wherein D is as defined in any one of the preceding claims.

13. The polymerisable monomer of any one of the preceding claims, wherein the polymerisable monomer has an oxidation potential for polymerisation of from about 0 to about 1.0V vs. Ag/AgCl (3 M KCl), for example from about 0.2 to 1.0, 0.3 to 1.0, 0.4 to 1.0, 0.5 to 1.0, 0.6 to 1.0, 0.7 to 1.0, 0.8 to 1.0, 0.2 to 0.9, 0.3 to 0.9, 0.4 to 0.9, 0.5 to 0.9, 0.6 to 0.9, 0.7 to 0.9, or 0.8 to 0.9V vs. Ag/AgCl (3 M KCl).

14. The polymerisable monomer of any one of the preceding claims, wherein the linker group has the formula:
—X¹—[(CH₂)ₘ—X²]ₓ—(CH₂)ₙ—X³—
wherein
x is an integer from 0 to 6;
m at each instance of x is independently an integer from 0 to 8;
n is an integer from 0 to 8;
X¹ and X² at each instance of x are each independently selected from the group consisting of a bond, —CH₂—, —CH═CH—, —O—, —S—, —N(R)—, —C(O)—, —C(O)O—, —OC(O)—, —N(R)C(O)—, —C(O)N(R)—, —SC(O)—, —C(O)S—, —NRSO₂—, —SO₂NR—, and —N(R)C(O)N(R)—;
R at each instance is independently hydrogen or alkyl;
X³ is a functional group through which the probe is attached;
provided that the linker group, excluding X³, is not more than 10 atoms in length.

15. The polymerisable monomer of any one of the preceding claims, wherein X³ is selected from the group consisting of —C(═NH)NH—, —NHC(═NH)—, —C(O)NH—, —NHC(O)—, —C(O)O—, —OC(O)—, —NHC(O)CR^{v}R^{w}S—, —SCR^{w}R^{v}C(O)NH—, —SS—, —C(O)NHN═CH—, —CH═NNHC(O)—, —CH═N—, —N═CH—, 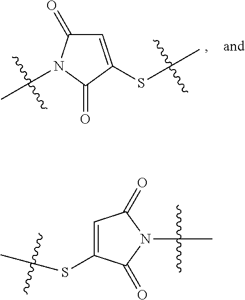
wherein R^{v} and R^{w} are at each instance independently H or C1-6 alkyl.

16. The polymerisable monomer of any one of the preceding claims, wherein the linker group is —O—(CH₂)ₘ—C(O)NH—, wherein m is an integer from 2 to 8.

17. The polymerisable monomer of any one of the preceding claims, wherein the probe is capable of binding one or more nucleic acids in a sequence specific manner.

18. The polymerisable monomer of any one of the preceding claims, wherein the probe comprises a single or double stranded oligonucleotide, polynucleotide, or an analogue thereof.

19. The polymerisable monomer of any one of the preceding claims, wherein the probe comprises an aptamer.

20. A conducting polymer comprising a monomer unit of the formula (2): 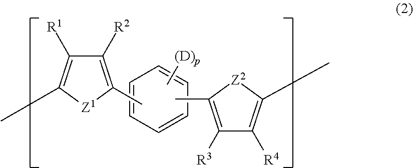
wherein p, R¹, R², R³, R⁴, D, Z¹, and Z² are as defined in any one of the preceding claims.

21. The conducting polymer of claim 20, wherein the conducting polymer comprises a monomer unit of formula (2A): 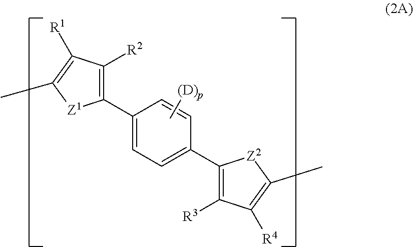
wherein p, R¹, R², R³, R⁴, D, Z¹, and Z² are as defined in any one of the preceding claims.

22. The conducting polymer of claim 20 or 21, wherein the conducting polymer comprises a monomer unit of formula (2B): 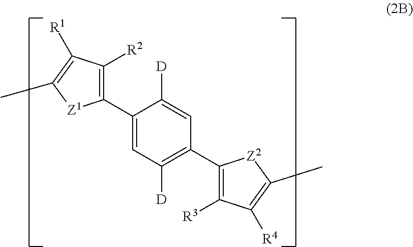
wherein R¹, R², R³, R⁴, D, Z¹, Z² and R^{a} are as defined in any one of the preceding claims.

23. The conducting polymer of any one of claims 20 to 22, wherein the conducting polymer comprises a monomer unit of formula (2C) or (2D): 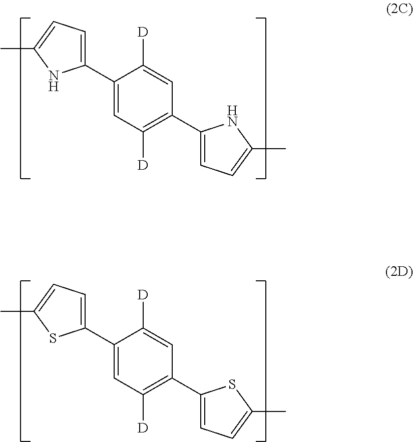
wherein D is as defined in any one of the preceding claims.

24. The conducting polymer of any one of claims 20 to 23, wherein the conducting polymer further comprises at least one monomer unit different to the monomer unit of the formula (2).

25. The conducting polymer of any one of claims 20 to 24, wherein the conducting polymer further comprises a monomer unit of formula (3), (4), (5), or a mixture of any two or more thereof:

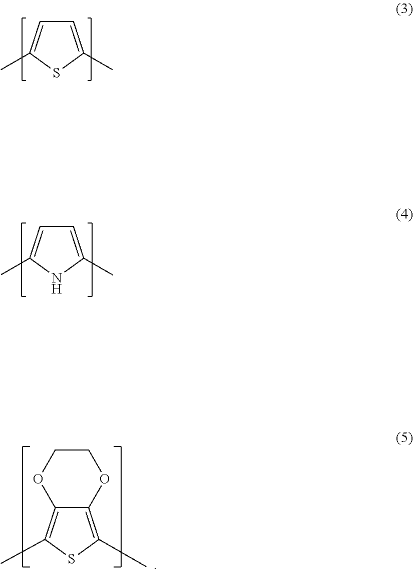

26. The conducting polymer of any one of claims 20 to 25, wherein the conducting polymer further comprises a monomer unit of formula (6): 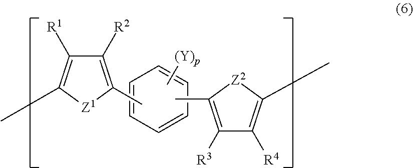
wherein
p, R¹, R², R³, R⁴, Z¹, and Z² are as defined in any one of the preceding claims; and
Y at each instance of p is independently selected from the group consisting of a water solubilising and/or protein repellent group, hydrogen, alkoxy, polyether, polyether alcohol, alkyl, alkenyl, cycloalkyl, cycloalkenyl, aryl, arylalkyl, heterocyclyl, and heteroaryl, wherein each alkyl, alkenyl, cycloalkyl, cycloalkenyl, aryl, arylalkyl, heterocyclyl, and heteroaryl is optionally substituted with one or more substituents independently selected from halo, nitro, nitrile, —C(O)R⁵, —OR⁵, —C(O)OR⁵, —OC(O)R⁵, —NR⁵R⁵, —C(O)NR⁵R⁵, —NR⁵C(O)R⁵, —NR⁵C(O)NR⁵R⁵, and alkyl.

27. The conducting polymer of claim 26, wherein Y at each instance of p is independently polyether.

28. The conducting polymer of claim 26 or 27, wherein the monomer unit of the formula (2) and monomer unit of the formula (6) are identical except for the D and Y groups.

29. The conducting polymer of any one of claims 26 to 28, wherein the ratio of the monomer unit of formula (2) to the monomer unit of the formula (6) is from about 10:1 to 1:1,000, 10:1 to 1:500, 10:1 to 1:100, 1:1 to 1:100, 1:1 to 1:50, 1:1 to 1:5, or 1:2 to 1:4, or about 1:3.

30. A method of making a conducting polymer as defined in any one of claims 20 to 29, the method comprising:
(a) providing a polymerisable monomer of the formula (1) as defined in any one of claims 1 to 19, and
(b) polymerising the monomer to provide a conducting polymer as defined in any one of claims 20 to 29.

31. The method of claim 30, comprising co-polymerising the polymerisable monomer of formula (1) and at least one additional polymerisable monomer different to the monomer of formula (1) to provide the conducting polymer.

32. The method of claim 30 or 31, comprising co-polymerising the polymerisable monomer of formula (1) and thiophene, pyrrole, 3,4-ethylenedioxythiophene (EDOT), or a mixture of any two or more thereof.

33. The method of any one of claims 30 to 32, comprising co-polymerising the polymerisable monomer of formula (1) and a polymerisable monomer of formula (7): 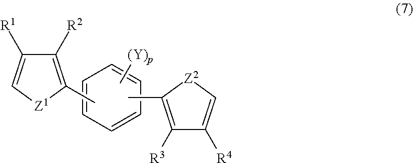
wherein p, R¹, R², R³, R⁴, Z¹, Z² and Y are as defined in any one of the preceding claims.

34. A conducting polymer made by a method of any one of claims 30 to 33.

35. A method of making a sensor comprising:
(i) providing a monomer of the formula (1) as defined in any one of claims 1 to 19;
(ii) providing a substrate; and
(iii) polymerising the monomer of the formula (1) as defined in any one of claims 1 to 19 to provide a conducting polymer as defined in any one of claims 20 to 29 and depositing the conducting polymer on a surface of the substrate to provide a coating of the conducting polymer on the surface of the substrate; or
(iii) depositing the monomer of the formula (1) as defined in any one of claims 1 to 19 on a surface of the substrate and polymerising the monomer to provide a coating of a conducting polymer as defined in any one of claims 20 to 29 on the surface of the substrate.

36. The method of claim 35, wherein the method comprises:
(i) providing a plurality of monomers of the formula (1) as defined in any one of claims 1 to 19;
(ii) providing a substrate; and
(iii) polymerising each monomer of the formula (1) to provide a conducting polymer and depositing each conducting polymer at a separate, predetermined location on a surface of the substrate to provide a coating of the conducting polymer at the location; or
(iii) depositing each monomer of the formula (1) at a separate, predetermined location on a surface of the substrate and polymerising each monomer to provide a coating of the conducting polymer at the location;
wherein at least two locations on the surface of the substrate are coated with a conducting polymer having a different probe.

37. The method of any one of claim 35 or 36, wherein the method comprises:
(i) providing a plurality of monomers of the formula (1) as defined in any one of claims 1 to 19;
(ii) providing a substrate comprising a plurality of electrodes; and
(iii) polymerising each monomer of the formula (1) to provide a conducting polymer and depositing each conducting polymer on a surface of a different electrode to provide a coating of the conducting polymer on the surface of the electrode; or
(iii) depositing each monomer of the formula (1) on a surface of a different electrode and polymerising each monomer to provide a coating of the conducting polymer on the surface of the electrode;
wherein the surfaces of at least two of the electrodes are coated with a conducting polymer having a different probe.

38. The method of any one of claims 35 to 37, wherein the monomer(s) are deposited on the surface of the substrate or electrode and polymerised to provide a coating of the conducting polymer on the surface of the substrate or electrode.

39. The method of any one of claims 35 to 38, wherein the monomer(s) are polymerised by electroless oxidative polymerisation, wherein the oxidant is oxygen or hydrogen peroxide.

40. The method of claim 39, wherein the electroless oxidative polymerisation is catalysed by an oxygen or hydrogen peroxide reduction catalyst.

41. The method of claim 40, wherein the catalyst comprises Pt, Pd, Ru, or Ir; an oxide of Pt, Pd, Ru, or Ir; carbon (for example carbon nanotubes, fullerines, or graphene); or a mixture of any two or more thereof.

42. The method of any one of claims 35 to 41 wherein the monomer(s) are stable to oxidative polymerisation by oxygen or hydrogen peroxide in the absence of an oxygen or hydrogen peroxide reduction catalyst for at least 4, 8, 12, 24 or 48 hours.

43. The method of any one of claims 40 to 42, wherein the surface of the substrate or electrode on which the conducting polymer(s) or monomer(s) are deposited consists of or comprises the catalyst.

44. The method of any one of claims 39 to 43, wherein the electroless oxidative polymerisation is of monomer(s) wherein Z¹ and Z² are each S.

45. The method of any one of claims 39 to 44, wherein the electroless oxidative polymerisation provides a polymer film having a thickness of from about 5 nm to 10 μm, preferably from 5 nm to 100 nm, when carried out for a period of time from about 1 second to about 120 seconds.

46. The method of any one of claims 35 to 38, wherein the monomer(s) are polymerised by electropolymerisation.

47. The method of claim 46, wherein the electropolymerisation is carried out at a potential of about 0 to about 1.0V vs. Ag/AgCl (3 M KCl), for example from about 0.2 to 1.0, 0.3 to 1.0, 0.4 to 1.0, 0.5 to 1.0, 0.6 to 1.0, 0.7 to 1.0, 0.8 to 1.0, 0.2 to 0.9, 0.3 to 0.9, 0.4 to 0.9, 0.5 to 0.9, 0.6 to 0.9, 0.7 to 0.9, or 0.8 to 0.9V vs. Ag/AgCl (3 M KCl).

48. The method of any one of claim 46 or 47, wherein the electropolymerisation provides a polymer film having a thickness of from about 5 nm to 10 μm, preferably from 5 nm to 100 nm, when carried out for a period of time from about 0.1 seconds to about 10 seconds.

49. A sensor comprising a substrate having a surface coated with a conducting polymer according to any one of claims 20 to 29.

50. The sensor of claim 49, wherein the substrate comprises at least one electrode having a surface coated with a conducting polymer according to any one of claims 20 to 29.

51. The sensor according of claim 49 or 50, wherein the substrate comprises a plurality of electrodes, each electrode comprising a surface coated with a conducting polymer according to any one of claims 20 to 29, wherein the surfaces of at least two of the electrodes are coated with a conducting polymer having a different probe.

52. A sensor system comprising a sensor according to any one of claims 49 to 51 and a detector for determining the presence or absence or amount of a target, for example a detector capable of detecting binding of a target by a probe.

53. The sensor system of claim 52, wherein the detector is capable of measuring an electrochemical property of the conducting polymer.

54. The sensor system of any of claim 52 or 53, wherein the detector is capable of measuring the impedance of the conducting polymer.

55. The sensor system of any of claims 52 to 54, wherein the system further comprises a redox couple.

56. The sensor system of claim 55, wherein the redox couple is ferro-ferricyanide.

57. The sensor or sensor system of any one of claims 49 to 56, wherein the sensor or sensor system further comprises a counter electrode and optionally a reference electrode.

58. The method, sensor, or sensor system of any one of claims 37 to 57, wherein the electrode(s) on which the conducting polymer(s) or monomers(s) are deposited or on which the conducting polymer(s) are coated is a gold (e.g. screen printed gold), platinum, carbon (e.g. glassy or screen printed carbon), stainless steel, indium tin oxide (ITO), or doped silicon wafer electrode.

59. A method for amplifying a target nucleic acid, the method comprising the steps of
a) providing a reaction volume comprising
(i) a first electrode comprising an electrochemically-active conducting polymer according to any one of claims 20 to 29, wherein the monomer unit of the formula (2) in the conducting polymer comprises a first single-stranded nucleic acid molecule capable of hydridizing to a first portion of a target nucleic acid sequence, and
(iii) a second electrode;
b) providing a reaction mixture to the reaction volume, the reaction mixture comprising
(i) a sample comprising the target nucleic acid,
(ii) a second single-stranded nucleic acid molecule comprising a nucleic acid sequence complementary to a second portion of the target nucleic acid sequence,
(iii) a nucleic acid polymerase,
(iv) a redox couple, and
(v) a supply of reagents for a nucleic acid amplification reaction;
c) performing a polymerase chain reaction, and
d) measuring the impedance of the first electrode at least once during the polymerase chain reaction.

60. The method of claim 59, wherein the reaction mixture comprises a second single-stranded nucleic acid molecule comprising a nucleic acid sequence complementary to a second portion of the target nucleic acid sequence.

61. The method of any one of claim 59 or claim 60 wherein the reaction mixture comprises the first single-stranded nucleic acid molecule, or a single-stranded nucleic acid molecule capable of hydridizing to the first portion of the target nucleic acid sequence.

62. The method of any one of claims 59 to 61, wherein the method comprises the additional step of determining the presence or amount of polynucleotide in the reaction volume on the basis of the one or more impedance measurements.

63. The method of any of claims 59 to 62, wherein the method comprises the additional step of measuring the impedance of the first electrode before the first elongation step of the nucleic acid amplification reaction.

64. The method of any one of claims 59 to 63, wherein the impedance is measured continuously throughout at least a portion of the polymerase chain reaction.

65. The method of any one of claims 59 to 64, wherein the method comprises measuring cumulative charge passed through the electrode.

66. The method of claim 65, wherein the method comprises measuring cumulative charge passed through the electrode and terminating the polymerisation on the basis of the measurement.

67. The method of claim 66, wherein the method comprises measuring cumulative charge passed through the electrode and terminating the polymerisation when a total charge of from about 1.0×10⁻⁵ C to about 5×10⁻⁵ C is measured.

68. The method of any one of claims 59 to 67, wherein the redox couple is a ferro-ferricyanide.

69. The method of any one of claims 59 to 68, wherein the target nucleic acid is present at an initial concentration of less than 1 pg/mL.

70. The method of any one of claims 59 to 69, wherein the target nucleic acid is present at an initial concentration of less than 1 fg/mL.

71. An apparatus for real-time nucleic acid amplification, the apparatus comprising
a reaction volume comprising
(i) a first electrode comprising an electrochemically-active conducting polymer according to any one of claims 20 to 29, wherein the monomer unit of the formula (2) in the conducting polymer comprises a first single-stranded nucleic acid molecule capable of hydridizing to a first portion of a target nucleic acid sequence, and
(iii) a second electrode;
wherein the reaction volume is suitable for containing a sample comprising nucleic acid, and wherein the reaction volume includes a heater or is adapted to engage with a thermocycler suitable for PCR.

72. The apparatus of claim 71, additionally comprising a thermocycler suitable for PCR.

73. The apparatus of claim 71 or claim 72, additionally comprising a device for measuring the impedance of at least the first electrode.

74. The apparatus of claim 73, wherein the device for device for measuring impedance is an LCR meter or is a potentiostat.

75. A system for amplifying a target nucleic acid in a sample, the system comprising
a) a reaction volume comprising
(i) a first electrode comprising an electrochemically-active conducting polymer according to any one of claims 20 to 29, wherein the monomer unit of the formula (2) of the conducting polymer comprises a first single-stranded nucleic acid molecule capable of hydridizing to a first portion of a target nucleic acid sequence, and
(iii) a second electrode;
b) optionally a reaction mixture comprising one or more of
(i) a second single-stranded nucleic acid molecule comprising a nucleic acid sequence complementary to a second portion of the target nucleic acid sequence,
(iii) a nucleic acid polymerase,
(iv) a redox couple, and
(v) a supply of reagents for a nucleic acid amplification reaction;
c) a device for measuring the impedance of at least the first electrode; and
d) a thermocycler.

76. The system of claim 75, wherein the device for measuring impedance is an LCR meter, a potentiostat, or the device measures impedance by determining the transconductance of or at the first electrode or by cyclic voltammetry.

77. A method for determining the presence or absence or amount of a target in a sample, the method comprising:
(a) contacting
(1) a sample which may comprise a target, and
(2) a sensor or sensor system according to any one of claims 49 to 58; and
(b) determining the presence or absence or amount of the target in the sample.

78. The method of claim 77, wherein determining the presence or absence or amount of a target in a sample comprises detecting binding of the target when present in a sample by a probe.

79. The method of claim 77 or 78, wherein the method comprises amplifying a target nucleic acid in the sample by a method according to any one of claims 59 to 70.

80. The method according to any one of claims 77 to 79, wherein the presence or absence or amount of the target in the sample is determined electrochemically or binding of the target is detected electrochemically.

81. The method according to any one of claims 77 to 80, wherein the presence or absence or amount of the target in the sample is determined by electrochemical impedance spectroscopy or binding of the target is detected by electrochemical impedance spectroscopy.

82. The method according to any one of claims 77 to 81, wherein the method comprises contacting the sample and the sensor in the presence of a redox couple.

83. The method of any one of claims 77 to 82, wherein the redox couple is ferro-ferricyanide.

84. The method, apparatus, or system of any one of claims 59 to 83, wherein the sample comprises double stranded nucleic acid.

85. The method, apparatus, or system of any one of claims 59 to 84, wherein the sample comprises genomic nucleic acid.

86. The method, apparatus, or system of any one of claims 59 to 85, wherein the sample comprises a lysate.

87. The method, apparatus, or system of claim 86, wherein the lysate is a cell lysate.

88. The method, apparatus, or system of claim 87, wherein the cell lysate is a bacterial cell lysate.

89. The method, apparatus, or system of any one of claims 59 to 87, wherein the sample or lysate comprises nucleic acid, preferably genomic nucleic acid, protein, lipids and other components, for example cellular components, produced by lysis.

90. The method, apparatus, or system of any one of claims 59 to 88, wherein the sample comprises a lysate from which at least a portion of solid components or particles produced by lysis have been removed.

91. The method, apparatus, or system of any one of claims 59 to 89, wherein the sample has not been subjected to nucleic acid extraction and/or purification.

92. The method, apparatus, or system of claim 90, wherein the sample has not been subjected to a nucleic acid extraction and/or purification comprising treatment with a proteinase, treatment with one or more organic solvents, precipitation of the nucleic acid, and/or purification and/or isolation of the precipitated nucleic acid.

93. The method, apparatus, or system of any one of claims 59 to 91, wherein the sample comprises a double stranded nucleic acid.

94. The method of claim 92, wherein the method comprises:
heating the sample for a period at a temperature sufficient to dissociate the nucleic acid strands, and
contacting the dissociated nucleic acid strands with the sensor or sensor system of the present invention, and
cooling to anneal the target nucleic acid with a probe of the sensor or sensor system.

95. The method, apparatus, or system of any one of claims 59 to 91, wherein the sample comprises microbes (for example, cells, such as bacteria, or viruses) comprising a target nucleic acid.

96. The method of claim 95, wherein the method comprises:
lysing the microbes,
heating the sample for a period at a temperature sufficient to dissociate double stranded nucleic acid contained therein,
contacting the dissociated nucleic acid strands with the sensor or sensor system of the present invention, and
cooling to anneal the target nucleic acid with a probe of the sensor or sensor system.

97. The method of claim 96, wherein the method comprises heating the sample for a period at a temperature sufficient to lyse the microbes and dissociate double stranded nucleic acid contained therein.

98. The method of any one of claims 46, 47, 58, wherein the electropolymerisation provides a polymer film having a thickness of from about 5 nm to 10 μm, preferably from 5 nm to 100 nm, when carried out for a period of time from about 0.1 seconds to about 30 seconds.


SEQUENCE LISTING (11 sequences)

SEQ ID NO: 1 - DNA, 24 bp, Artificial
  Synthetic
ggtctagctacagagaaatctcga

SEQ ID NO: 2 - DNA, 24 bp, Artificial
  Synthetic
cagtagacgggggtgtcacgcgac

SEQ ID NO: 3 - DNA, 24 bp, Artificial
  Synthetic
catctttgggctgttttcttccgc

SEQ ID NO: 4 - DNA, 22 bp, Artificial
  Synthetic
ctagtttagacagctaggaagg

SEQ ID NO: 5 - DNA, 24 bp, Artificial
  Synthetic
tcgagatttctctgtagctagacc

SEQ ID NO: 6 - DNA, 24 bp, Artificial
  Synthetic
tcgagatttctcagtagctagacc

SEQ ID NO: 7 - DNA, 24 bp, Artificial
  Synthetic
tcgagatttctctctagctagacc

SEQ ID NO: 8 - DNA, 24 bp, Artificial
  Synthetic
gtcatctgcccccacagagcgctg

SEQ ID NO: 9 - DNA, 24 bp, Artificial
  Synthetic
gcggaagaaaacagcccaaagatg

SEQ ID NO: 10 - DNA, 22 bp, Artificial
  Synthetic
ccttcctagctgtctaaactag

SEQ ID NO: 11 - DNA, 12 bp, Escherichia coli
ctagtttagaca